(19) United States
(12) Patent Application Publication
(10) Pub. No.: US 2003/0182672 A1
(43) Pub. Date: Sep. 25, 2003

(54) Genetic silencing

(76) Inventors: Michael Wayne Graham, Chapel Hill (AU); Robert Norman Rice, Sinnamon Park (AU); Kenneth Clifford Reed, St. Lucia (AU); Kathleen Margaret Murphy, Rocklea (AU)

Correspondence Address: Michael R. Ward, Morrison & Foerster LLP, 425 Market Street, San Francisco, CA, 94105-2482, US

(21) Appl. No.: 10/245,805

(22) Filed: Sep. 16, 2002

Related U.S. Application Data
(63) Continuation of application No. PCT/AU01/00297, filed on Mar. 16, 2001.

(30) Foreign Application Priority Data
Sep. 27, 2001 (WO) 01/70949
Mar. 17, 2000 (AU) PQ 6363
Jan. 24, 2001 (AU) PR 2700

(51) Int. Cl. A01K 67/027; C12P 21/02; C12N 5/06; C12N 5/08; C12N 15/85

(52) U.S. Cl. 800/18; 435/69.1; 435/320.1; 435/325; 435/372; 435/455

(57) ABSTRACT

A method of inducing, promoting or otherwise facilitating a change in the phenotype of an animal cell or group of animal cells including an animal comprising said cells. The modulation of phenotypic expression is conveniently accomplished via genotypic manipulation through such means as reducing translation of transcript to proteinaceous product. The ability to induce, promote or otherwise facilitate the silencing of expressible genetic sequences provides a means for modulating the phenotype in, for example, the medical, veterinary and the animal husbandry industries. Expressible genetic sequences contemplated by the present invention include not only genes normally resident in a particular animal cell (i.e., indigenous genes) but also genes introduced through recombinant means or through infection by pathogenic agents such as viruses.

### FIELD OF THE INVENTION

The present invention relates generally to a method of inducing, promoting or otherwise facilitating a change in the phenotype of an animal cell or group of animal cells including a animal comprising said cells. The modulation of phenotypic expression is conveniently accomplished via genotypic manipulation through such means as reducing translation of transcript to proteinaceous product. The ability to induce, promote or otherwise facilitate the silencing of expressible genetic sequences provides a means for modulating the phenotype in, for example, the medical, veterinary and the animal husbandry industries. Expressible genetic sequences contemplated by the present invention including not only genes normally resident in a particular animal cell (i.e. indigenous genes) but also genes introduced through recombinant means or through infection by pathogenic agents such as viruses.

### BACKGROUND OF THE INVENTION

Reference to any prior art in this specification is not, and should not be taken as, an acknowledgment or any form of suggestion that this prior art forms part of the common general knowledge in Australia or any other country.

Bibliographic details of the publications referred to by author in this specification are collected at the end of the description.

The increasing sophistication of recombinant DNA techniques is greatly facilitating research and development in the medical and veterinary industries. One important aspect of recombinant DNA technology is the development of means to alter the genotype by modulating expression of genetic material. A myriad of desirable phenotypic traits are potentially obtainable following selective inactivation of gene expression.

Gene inactivation, that is, the inactivation of gene expression, may occur in cis or in trans. For cis inactivation, only the target gene is inactivated and other similar genes dispersed throughout the genome are not affected. In contrast, inactivation in trans occurs when one or more genes dispersed throughout the genome and sharing homology with a particular target sequence are also inactivated. In the literature, the term “gene silencing” is frequently used. However, this is generally done without an appreciation of whether the gene silencing events are capable of acting in trans or in cis. This is relevant to the commercial exploitation of gene silencing technology since cis inactivation events are of less usefulness than events in trans. For example, there is less likelihood of success in targeting endogenous genes (e.g. plant genes) or exogenous genes (e.g. genes from pathogens) using techniques which promote cis inactivation. Furthermore, in instances where gene inactivation is monitored using a marker gene, it is frequently not possible to discriminate between cis and trans inactivation events. There is, therefore, confusion in the literature regarding the precise molecular mechanisms of gene inactivation (Garrick et al., 1998; Pal-Bahdra et al., 1997; Bahramian and Zarbl, 1999).

The existing literature is extremely confused as to mechanisms of gene inactivation or gene silencing. For example, the term “antisense” is used to describe situations where genetic constructs designed to express antisense RNAs are introduced into a cell, the aim being to decrease expression of that particular RNA. This strategy has been widely used experimentally and in practical applications. The mechanism by which antisense RNAs function is generally believed to involve duplex formation between the endogenous sense RNA and the antisense sequences which inhibits translation. There is, however, no unequivocal evidence that this mechanism occurs at all in higher eukaryotic systems.

The term “gene silencing” is frequently used to describe inactivation of the expression of a transgene in eukaryotic cells. There is much confusion in the literature as to the mechanism by which this occurs, although it is generally believed to result from transcriptional inactivation. It is unclear whether this particular mechanism has any great practical utility since the expression of the gene itself is inactivated, i.e. there is no trans inactivation of other genes.

In plants, the term “co-suppression” is used to describe precisely situations where a transgene is introduced stably into the genome and expressed as a sense RNA. Surprisingly, expression of such transgene sequences results in inactivation of homologous genes, i.e. a sequence specific trans inactivation of gene expression (Napoli et al., 1990; van der Krol et al., 1990). The molecular phenotype of cells in which this occurs is well described in plant systems: a gene is transcribed as a precursor mRNA, but it is not translated. Another term used to describe co-suppression is post-transcriptional gene inactivation. The disappearance of mRNA sequences is thought to occur as a consequence of activation of a sequence specific RNA degradative system (Lindbo et al., 1993; Waterhouse et al, 1999). There is considerable confusion within the animal literature regarding the term “co-suppression” (Bingham, 1997).

Co-suppression, as defined by the specific molecular phenotype of gene transcription without translation, has previously been considered not to occur in mammalian systems. It has been described only in plant systems and a lower eukaryote, Neurospera (Cogoni et al., 1996; Cogoni and Macino, 1997).

In work leading up to the present invention, the inventors have employed genetic manipulative techniques to induce gene silencing in animal cells. The genetic manipulative techniques involve the induction of post-trnnscriptional inactivation events. The inventors have thereby provided a means for co-suppression in animal cells. The induction of co-suppression in animal cells permits the manipulation of a range of phenotypes in animals.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Nucleotide and amino acid sequences are referred to by a sequence identifier number (SEQ ID NO:). The SEQ ID NOs: correspond numerically to the sequence identifiers <400>1, <400>2, etc. A sequence listing is provided after the claims.

One aspect of the present invention provides a genetic construct comprising a sequence of nucleotides substantially identical to a target endogenous sequence of nucleotides in the genome of a vertebrate animal cell wherein upon introduction of said genetic construct to said animal cell, an RNA transcript resulting from transcription of a gene comprising said endogenous target sequence of nucleotides exhibits an altered capacity for translation into a proteinaceous product.

Another aspect of the present invention provides a genetic construct comprising:—

(i) a nucleotide sequence substantially identical to a target endogenous sequence of nucleotides in the genome of a vertebrate animal cell;

(ii) a single nucleotide sequence substantially complementary to said target endogenous nucleotide sequence defined in (i);

(iii) an intron nucleotide sequence separating said nucleotide sequence of (i) and (ii);

wherein upon introduction of said construct to said animal cell, an RNA transcript resulting from transcription of a gene comprising said endogenous target sequence of nucleotides exhibits an altered capacity for transcription.

A further aspect of the present invention provides a genetic construct comprising:—

(i) a nucleotide sequence substantially identical to a target endogenous sequence of nucleotides in the genome of a vertebrate animal cell;

(ii) a nucleotide sequence substantially complementary to said target endogenous nucleotide sequence defined in (i);

(iii) an intron nucleotide sequence separating said nucleotide sequence of (i) and (ii);

wherein upon introduction of said construct to said animal cell, an RNA transcript resulting from transcription of a gene comprising said endogenous target sequence of nucleotides exhibits an altered capacity for translation into a proteinaceous product and wherein there is substantially no reduction in the level of transcription of said gene comprising the endogenous target sequence and/or total level of RNA transcribed from said gene comprising said endogenous target sequence of nucleotides is not substantially reduced.

Yet another aspect of the present invention provides a genetically modified vertebrate animal cell characterized in that said cell:—

(i) comprises a sense copy of a target endogenous nucleotide sequence introduced into said cell or a parent cell thereof;

(ii) comprises substantially no proteinaceous product encoded by a gene comprising said endogenous target nucleotide sequence compared to a non-genetically modified form of same cell; and

(iii) comprises substantially no reduction in the levels of steady state total RNA relative to a non-genetically modified form of the same cell.

Another aspect of the present invention provides a method of altering the phenotype of a vertebrate animal cell wherein said phenotype is conferred or otherwise facilitated by the expression of an endogenous gene, said method comprising introducing a genetic construct into said cell or a parent of said cell wherein the genetic construct comprises a nucleotide sequence substantially identical to a nucleotide sequence comprising said endogenous gene or part thereof and wherein a transcript exhibits an altered capacity for translation into a proteinaceous product compared to a cell without having had the genetic construct introduced.

Even yet another aspect of the present invention provides a genetically modified murine animal comprising a nucleotide sequence substantially identical to a target endogenous sequence of nucleotides in the genome of a cell of said murine animal wherein an RNA transcript resulting from transcription of a gene comprising said endogenous target sequence of nucleotides exhibits an altered capacity for translation into a proteinaceous product.

Still a further aspect of the present invention is directed to the use of genetic construct comprising a sequence of nucleotides substantially identical to a target endogenous sequence of nucleotides in the genome of a vertebrate animal cell in the generation of an animal cell wherein an RNA transcript resulting from transcription of a gene comprising said endogenous target sequence of nucleotides exhibits an altered capacity for translation into a proteinaceous product.

Another aspect of the present invention contemplates a method of genetic therapy in a vertebrate animal, said method comprising introducing into cells of said animal comprising a sequence of nucleotides substantially identical to a target endogenous sequence of nucleotides in the genome of said animal cells such that upon introduction of said nucleotide sequence, RNA transcript resulting from transcription of a gene comprising said endogenous target sequence of nucleotides exhibits an altered capacity for translation into a proteinaceous product.

### BRIEF DESCRIPTION OF THE FIGURES

FIG. 1 is a diagrammatic representation of the plasmid, pEGFP-N1. For further details, refer to Example 1.

FIG. 2 is a diagrammatic representation of the plasmid, pCMV.cass. For further details, refer to Example 11.

FIG. 3 is a diagrammatic representation of the plasmid, pCMV.BGI2.cass. For further details, refer to Example 11.

FIG. 4 is a diagrammatic representation of the plasmid, pCMV.GFP.BGI2.PFG. For further details, refer to Example 12.

FIG. 5 is a diagrammatic representation of the plasmid, pCMV.EGFP. For further details, refer to Example 12.

FIG. 6 is a diagrammatic representation of the plasmid, pCMV^{pur}.BGI2.cass. For further details, refer to Example 12.

FIG. 7 is a diagrammatic representation of the plasmid, pCMV^{pur}.GFP.BGI2.PFG. For further details, refer to Example 12.

FIG. 8 shows an example of Southern blot analysis of putative transgenic cell lines, in this instance porcine kidney cells (PK) which had been transformed with the construct pCMV.EGFP. Genomic DNA was isolated from PK-1 cells and transformed lines, digested with the restriction endonuclease BamH1 and probed with a ³²P-dCTp labeled EGFP DNA fragment. Lane A is a molecular weight marker where sizes of each fragment are indicated in kilobases (kb); Lane B is the parental cell line PK-1. Lane C is A4, a transgenic EGFP-expressing PK-1 cell line; Lane D is C9, a transgenic non-expressing PK-1 cell line.

FIG. 9 shows micrographs of PK-1 cell lines transformed with pCMV.EGFP, viewed under normal light and under fluorescence conditions designed to detect GFP. A: PK EGFP 2.11 cells under normal light; B: PK EGFP 2.11 cells under fluorescence conditions; C: PK EGFP 2.18 cells under normal light; D: PK EGFP 2.18 cells under fluorescence conditions.

FIG. 10 is a diagrammatic representation of the plasmid, pCMV.BEV2.BGI2.2VEB. For further details, refer to Example 13.

FIG. 11 is a diagrammatic representation of the plasmid, pCMV.BEV.EGFP.VEB. For further details, refer to Example 13.

FIG. 12 shows micrographs of CRIB-1 cells and a CRIB-1 transformed line [CRIB-1 BGI2 # 19(tol)] prior to and 48 hr after infection with identical titres of BEV. A: CRIB-1 cells prior to BEV infection; B: CRIB-1 cells 48 hr after BEV infection; C: CRIB-1 BGI2 # 19(tol) cells prior to infection with BEV; D: CRIB-1 BGI2 # 19(tol) 48 hr after BEV infection.For further details, refer to Example 13.

FIG. 13 is a diagrammatic representation of the plasmid, pCMV.TYR.BGI2.RYT. For further details, refer to Example 14.

FIG. 14 is a diagrammatic representation of the plasmid, pCMV.TYR. For further details, refer to Example 14.

FIG. 15 is a diagrammatic representation of the plasmid, pCMV.TYR.TYR. For further details, refer to Example 14.

FIG. 16 shows levels of pigmentation in B16 cells and B16 cells transformed with pCMV.TYR.BGI2.RYT. Cell lines are, from left to right: B16, B16 2.1.6, B16 2.1.11, B16 3.1.4, B16 3.1.15, B16 4.12.2 and B116 4.12.3. For further details, refer to Example 14.

FIG. 17 is a diagrammatic representation of the plasmid, pCMV.GALT.BGI2.TLAG. For further details, refer to Example 16.

FIG. 18 is a diagrammatic representation of the plasmid, pCMV. BGI2.KTM. For further details, refer to Example 17.

FIG. 19 is a diagrammatic representation of the piasmid, HER2.BGI2.2REH. For further details, refer to Example 18.

FIG. 20 shows immunoflourescent micrographs of MDA-MB468 cells and MDA-MB-468 cells transformed with pCMV.HER2.BGI2.2REH stained for HER-2. A: MDA-MB-468 cells; B: MDA-MB-468 cells stained with only the secondary antibody, C: MDA-MB-468 1.4 cells stained for HER-2; D: MDA-MB-468 1.10 cells stained for HER-2. For further details, refer to Example 18.

FIG. 21 shows FACS analyses of HER-2 expression in (A) MDA-MB-468 cells; (B) MDA-MB-468 1.4 cells; (C) MDA-MB-468 1.10 cells. For further details, refer to Example 18.

FIG. 22 is a diagrammatic representation of the plasmid, pCMV.BRN2.BGI2.2NRB. For further details, refer to Example 19.

FIG. 23 is a diagrammatic representation of the plasmid, pCMV.YB1.BGI2.1BY. For further details, refer to Example 20.

FIG. 24 is a diagrammatic representation of the plasmid, pCMV.YB1.p53.BGI2.35p. 1BY. For Further details, refer to Example 20.

FIG. 25 is a histograph showing viable cell counts after transfection with YB-1-related gene constructs and oligonucleotides. Viable cells were counted in quadruplicate samples with a haemocytometer following staining with trypan blue. Column heights show the average cell count of two independent transfection experiments and vertical bars indicate the standard deviation. (A) Viable B10.2 cell counts 72 hr after transfection with gene constructs: (i) control: pCMV.EGFP; (ii) pCMV.YB1.BGI2.1BY; (iii) pCMV.YB1.p53.BGI2.35p.1BY. All materials and procedures used are described in the text for Example 20. (B) Viable Para 212 cell counts 72 hr after transfection with gene constructs: (i) control: pCMV.EGFP; (ii) pCMV.YB1.BGI2.1BY; (iii) pCMV.YB1.p53.BGI2.35p.1BY. All materials and procedures used are described in the text for Example 20. (C) Viable B10.2 cell counts 18 hr after transfection with oligonucleotides: (i) control: Lipofectin (trademark) only, (ii) control: non-specific oligonucleotide; (iii) decoy Y-box oligonucleotide. All materials and procedures used are described in the text for Example 20. (D) Viable Pam 212 cell counts 18 hr after transfection with oligonucleotides: (i) control: Lipofectin (trademark) only; (ii) control: non-specific oligonucleotide; (iii) decoy Y-box oligonucleotide. All materials and procedures used are described in the text for Example 20.

### DETAILED DESCRIPTION OF THE PREFERRED EMBODIMENTS

The present invention is predicated in part on the use of sense nucleotide sequences relative to an endogenous nucleotide sequence in a vertebrate animal cell to down-regulate expression of a gene comprising said endogenous nucleotide sequence. The endogenous nucleotide sequence may comprise all or part of a gene and may or may not indigenous to the cell. A non-indigenous gene includes a gene in the animal cell introduced by, for example, viral infection or recombinant DNA technology. An indigenous gene includes a gene which would be considered to be naturally present in the animal cell. The down-regulation of a target endogenous gene includes the introduction of the sense nucleotide sequence to that particular cell or a parent of that cell.

Accordingly, one aspect of the present invention provides a genetic construct comprising a sequence of nucleotides substantially identical to a target endogenous sequence of nucleotides in the genome of a vertebrate animal cell wherein upon introduction of said genetic construct to-said animal cell, an RNA transcript resulting from transcription of a gene comprising said endogenous target sequence of nucleotides exhibits an altered capacity for translation into a proteinaceous product.

Reference to “altered capacity” preferably includes a reduction in the level of translation such as from about 10% to about 100% and more preferably from about 20% to about 90% relative to a cell which is not genetically modified. In a particularly preferred embodiment, the gene corresponding to the target endogenous sequence is substantially not translated into a proteinaceous product. Conveniently, an altered capacity of translation is determined by any change of phenotype wherein the phenotype, in a non-genetically modified cell is facilitated by the expression of said endogenous gene.

Preferably the vertebrate animal cells are derived from mammals, avian species, fish or reptiles. Preferably, the vertebrate animal cells are derived from mammals. Mammalian cells may be from a human, primate, livestock animal (e.g. sheep, cow, goat, pig, donkey, horse), laboratory test animal (e.g. rat, mouse, rabbit, guinea pig, hamster), companion animal (e.g. dog, cat) or captured wild animal. Particularly preferred mammalian cells are from human and murine animals.

The nucleotide sequence in the genome of a vertebrate animal cell is referred to as a “genomic” nucleotide sequence and preferably corresponds to a gene encoding a product conferring a particular phenotype on the animal cell, group of animal cells and/or an animal comprising said cells. As stated above, the endogenous gene may be indigenous to the animal cell or may be derived from a exogenous source such as a virus, intracellular parasite or introduced by recombinant or other physical means. Reference, therefore, to “genome” or “genomic” includes not only chromosomal genetic material but also extrachromosomal genetic material such as derived from non-integrated viruses. Reference to a “substantially identical” nucleotide sequence is also encompassed by terms including substantial homology and substantial similarity.

Reference herein to a “gene” is to be taken in its broadest context and includes:—

(i) a classical genomic gene consisting of transcriptional and/or translational regulatory sequences and/or a coding region and/or non-translated sequences (i.e. introns, 5′- and 3′-untranslated sequences);

(ii) mRNA or cDNA corresponding to the coding regions (i.e. exons) optionally comprising 5′- and 3′-untranslated sequences linked thereto; or

(iii) an amplified DNA fragment or other recombinant nucleic acid molecule produced in vitro and comprising all or a part of the coding region and/or 5′- or 3′-untranslated sequences linked thereto.

The gene in the animal cell genome is also referred to as a target gene or target sequence and may be, as stated above, naturally resident in the genome or may be introduced by recombinant techniques or other means, e.g. viral infection. The term “gene” is not to be construed as limiting the target sequence to any particular structure, size or composition.

The target sequence or gene is any nucleotide sequence which is capable of being expressed to form a mRNA and/or a proteinaceous product The term “expressed” and related terms such as “expression” include one or both steps of transcription and/or translation.

In a preferred embodiment, the nucleotide sequence in the genetic construct further comprises a nucleotide sequence complementary to the target endogenous nucleotide sequence.

Accordingly, another aspect of the present invention provides a genetic construct comprising:—

(i) a nucleotide sequence substantially identical to a target endogenous sequence of nucleotides in the genome of a vertebrate animal cell;

(ii) a single nucleotide sequence substantially complementary to said target endogenous nucleotide sequence defined in (i);

(iii) an intron nucleotide sequence separating said nucleotide sequence of (i) and (ii);

wherein upon introduction of said construct to said animal cell, an RNA transcript resulting from transcription of a gene comprising said endogenous target sequence of nucleotides exhibits an altered capacity for transcription.

Preferably, the identical and complementary sequences are separated by an intron sequence. An example of a suitable intron sequence includes but is not limited to all or part of a intron from a gene encoding β-globin such as human β-globin intron 2.

The loss of proteinaceous product is conveniently observed by the change (e.g. loss) of a phenotypic property or an alteration in a genotypic property.

The target gene may encode a structural protein or a regulatory protein. A “regulatory protein” includes a transcription factor, heat shock protein or a protein involved in DNA/RNA replication, transcription and/or translation. The target gene may also be resident in a viral genome which has integrated into the animal gene or is present as an extrachromosomal element. For example, the target gene may be a gene on an HIV genome. In this case, the genetic construct is useful in inactivating translation of the HIV gene in a mammalian cell.

Wherein the target gene is a viral gene, it is particularly preferred that the viral gene encodes a function which is essential for replication or reproduction of the virus, such as but not limited to a DNA polymerase or RNA polymerase gene or a viral coat protein gene, amongst others. In a particularly preferred embodiment, the target gene comprises an RNA polymerase gene derived from a single-stranded (+) RNA virus such as bovine enterovirus (BEV), Sinbis alphavirus or a lentivirus such as but not limited to an immunodeficiency virus (e.g. HIV-1) or alternatively, a DNA polymerase derived from a double-stranded DNA virus such as bovine herpes virus or herpes simplex virus I (HSVI), amongst others.

In a particularly preferred embodiment, the post-trnnscriptional inactivation is preferably by a mechanism involving trans inactivation.

The genetic construct of the present invention generally, but not exclusively, comprises a synthetic gene. A “synthetic gene” comprises a nucleotide sequence which, when expressed inside an animal cell down-regulates expression of a homologous gene, endogenous to the animal cell or an integrated viral gene resident therein.

A synthetic gene of the present invention may be derived from naturally-occurring genes by standard recombinant techniques, the only requirement being that the synthetic gene is substantially identical or otherwise similar at the nucleotide sequence level to at least a part of the target gene, the expression of which is to be modified. By “substantially identical” is meant that the structural gene sequence of the synthetic gene is at least about 80-90% identical to 30 or more contiguous nucleotides of the target gene, more preferably at least about 90-95% identical to 30 or more contiguous nueleotides of the target gene and even more preferably at least about 95-99% identical or absolutely identical to 30 or more contiguous nucleotides of the target gene. Alternatively, the gene is capable of hybridizing to a target gene sequence under low, preferably medium or more preferably high stringency conditions.

Reference herein to a low stringency includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions. Alternative stringency conditions may be applied where necessary, such as medium stringency, which includes and encompasses from at least about 16% v/v to at least about 30% v/v formamiide and from at least about 0.5 M to at least about 0.9 M salt for hybridization, and at least about 0.5 M to at least about 0.9 M salt for washing conditions, or high stringency, which includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out at Tₘ=69.3+0.41 (G+C) % (Marmur and Doty, 1962). However, the Tₘ of a duplex DNA decreases by 1° C. with every increase of 1% in the number of mismatch base pairs (B3onner and Laskey, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Generally, a synthetic gene of the instant invention may be subjected to mutagenesis to produce single or multiple nucleotide substitutions, deletions and/or additions without affecting its ability to modify target gene expression. Nucleotide insertional derivatives of the synthetic gene of the present invention include 5′ and 3′ terminal fusions as well as intra-sequence insertions of single or multiple nucleotides. Insertional nucleotide sequence variants are those in which one or more nucleotides are introduced into a predetermined site in the nucleotide sequence although random insertion is also possible with suitable screening of the resulting product. Deletional variants are characterized by the removal of one or more nucleotides from the sequence. Substitutional nucleotide variants are those in which at least one nucleotide in the sequence has been removed and a different nucleotide inserted in its place. Such a substitution may be “silent” in that the substitution does not change the amino acid defined by the codon. Alternatively, substituents are designed to alter one amino acid for another similar acting amino acid, or amino acid of like charge, polarity, or hydrophobicity.

Accordingly, the present invention extends to homologs, analogs and derivatives of the synthetic genes described herein.

For the present purpose, “homologs” of a gene as hereinbefore defined or of a nucleotide sequence shall be taken to refer to an isolated nucleic acid molecule which is substantially the same as the nucleic acid molecule of the present invention or its complementary nucleotide sequence, notwithstanding the occurrence within said sequence of one or more nucleotide substitutions, insertions, deletions, or rearrangements.

“Analogs” of a gene as hereinbefore defined or of a nucleotide sequence set forth herein shall be taken to refer to an isolated nucleic acid molecule which is substantially the same as a nucleic acid molecule of the present invention or its complementary nucleotide sequence, notwithstanding the occurrence of any non-nucleotide constituents not normally present in said isolated nucleic acid molecule, for example, carbohydrates, radiochemicals including radionucleotides, reporter molecules such as but not limited to DIG, alkaline phosphatase or horseradish peroxidase, amongst others.

“Derivatives” of a gene as hereinbefore defined or of a nucleotide sequence set forth herein shall be taken to refer to any isolated nucleic acid molecule which contains significant sequence similarity to said sequence or a part thereof.

Accordingly, the structural gene component of the synthetic gene may comprise a nucleotide sequence which is at least about 80% identical or homologous to at least about 30 contiguous nucleotides of an endogenous target gene, a foreign target gene or a viral target gene present in an animal cell or a homologue, analogue, derivative thereof or a complementary sequence thereto.

The genetic construct of the present invention generally but not exclusively comprises a nucleotide sequence, such as in the form of a synthetic gene, operably linked to a promoter sequence. Other components of the genetic construct include but are not limited to regulatory regions, transcriptional start or modifying sites and one or more genes encoding a reporter molecule. Further components able to be included on the genetic construct extend to viral components such as viral DNA polymerase and/or RNA polymerase. Non-viral components include RNA-dependent RNA polymerase. The structural portion of the synthetic gene may or may not contain a translational start site or 5′- and 3′-untranslated regions, and may or may not encode the fill length protein produced by the corresponding endogenous mammalian gene.

Another aspect of the present invention provides a genetic construct comprising a nucleotide sequence substantially homologous to a nucleotide sequence in the genome of a mammalian cell, said first-mentioned nucleotide sequence operably inked to a promoter, said genetic construct optionally further comprising one or more regulatory sequences and/or a gene sequence encoding a reporter molecule wherein upon introduction of said genetic construct into an animal cell, the expression of the endogenous nucleotide sequences having homology to the nucleotide sequence on the genetic construct is inhibited, reduced or otherwise down-regulated via a process comprising post-transcriptional modulation.

Reference herein to a “promoter” is to be taken in its broadest context and includes the transcriptional regulatory sequences of a classical genomic gene, including the TATA box which is required for accurate transcription initiation in eukaryotic cells, with or without a CCAAT box sequence and additional regulatory elements (i.e. upstream activating sequences, enhancers and silencers).

A promoter is usually, but not necessarily, positioned upstream or 5′, of the structural gene component of the synthetic gene of the invention, the expression of which it regulates. Furthermore, the regulatory elements comprising a promoter are usually positioned within 2 kb of the start site of transcription of the structural gene.

In the present context, the term “promoter” is also used to describe a synthetic or fusion molecule or derivative which confers, activates or enhances expression of an isolated nucleic acid molecule in a mammalian cell. Another or the same promoter may also be required to function in plant, animal, insect, fungal, yeast or bacterial cells. Preferred promoters may contain additional copies of one or more specific regulatory elements to further enhance expression of a structural gene, which in turn regulates and/or alters the spatial expression and/or temporal expression of the gene. For example, regulatory elements which confer inducibility on the expression of the structural gene may be placed adjacent to a heterologous promoter sequence driving expression of a nucleic acid molecule.

Placing a structural gene under the regulatory control of a promoter sequence means positioning said molecule such that expression is controlled by the promoter sequence. Promoters are generally positioned 5′ (upstream) to the genes that they control. In the construction of heterologous promoter/structural gene combinations, it is generally preferred to position the promoter at a distance from the gene transcription start site that is approximately the same as the distance between that promoter and the gene it controls in its natural setting, i.e. the gene from which the promoter is derived. As is known in the art, some variation in this distance can be accommodated without loss of promoter function. Similarly, the preferred positioning of a regulatory sequence element with respect to a heterologous gene to be placed under its control is defined by the positioning of the element in its natural setting, ire. the genes from which it is derived. Again, as is known in the art, some variation in this distance can also occur.

The promoter may regulate the expression of the structural gene component constitutively, or differentially with respect to the cell, tissue or organ in which expression occurs, or with respect to the developmental stage at which expression occurs, or in response to stimuli such as physiological stresses, regulatory proteins, hormones, pathogens or metal ions, amongst others.

Preferably, the promoter is capable of regulating expression of a nucleic acid molecule in a mammalian cell, at least during the period of time over which the target gene is expressed therein and more preferably also immediately preceding the commencement of detectable expression of the target gene in said cell. Promoters may be constitutive, inducible or developmentally regulated.

In the present context, the terms “in operable connection with” or “operably under the control” or similar shall be taken to indicate that expression of the structural gene is under the control of the promoter sequence with which it is spatially connected in a cell.

The genetic construct of the present invention may also comprise multiple nucleotide sequences each optionally operably linked to one or more promoters and each directed to a target gene within &e animal cell.

A multiple nucleotide sequence may comprise a tandem repeat or concatemer of two or more identical nucleotide sequences or alternatively, a tandem array or concatemer of non-identical nucleotide sequences, the only requirement being that each of the nucleotide sequences contained therein is substantially identical to the target gene sequence or a complementary sequence thereto. In this regard, those skilled in the art will be aware that a cDNA molecule may also be regarded as a multiple structural gene sequence in the context of the present invention, insofar as it comprises a tandem array or concatemer of exon sequences derived from a genomic target gene. Accordingly, cDNA molecules and any tandem array, tandem repeat or concatemer of exon sequences and/or intron sequences and/or 5′-untranslated and/or 3′-untranslated sequences are clearly encompassed by this embodiment of the invention.

Preferably, the multiple nucleotide sequences comprise at least 2-8 individual structural gene sequences, more preferably at least about 2-6 individual structural gene sequences and more preferably at least about 2-4 individual structural gene sequences.

The optimum number of structural gene sequences which may be involved in the synthetic gene of the present invention will vary considerably, depending upon the length of each of said structural gene sequences, their orientation and degree of identity to each other. For example, those skilled in the art will be aware of the inherent instability of palindromic nucleotide sequences in vivo and the difficulties associated with constructing long synthetic genes comprising inverted repeated nucleotide sequences, because of the tendency for such sequences to form hairpin loops and to recombine in vivo. Notwithstanding such difficulties, the optimum number of structural gene sequences to be included in the synthetic genes of the present invention may be determined empirically by those skilled in the art, without any undue experimentation and by following standard procedures such as the construction of the synthetic gene of the invention using recombinase-deficient cell lines, reducing the number of repeated sequences to a level which eliminates or minimizes recombination events and by keeping the total length of the multiple structural gene sequence to an acceptable limit, preferably no more than 5-10 kb, more preferably no more than 2-5 kb and even more preferably no more than 0.5-2.0 kb in length.

In one embodiment, the effect of the genetic contruct including synthetic gene comprising the sense nucleotide sequence is to reduce translation of transcript to proteinaceous product while not substantially reducing the level of transcription of the target gene. Alternatively or in addition to, the genetic construct including synthetic gene does not result in a substantial reduction in steady state levels of total RNA.

Accordingly, a particularly preferred embodiment of the present invention provides a genetic construct comprising:—

(i) a nucleotide sequence substantially identical to a target endogenous sequence of nucleotides in the genome of a vertebrate animal cell;

(ii) a nucleotide sequence substantially complementary to said target endogenous nucleotide sequence defined in (i);

(iii) an intron nucleotide sequence separating said nucleotide sequence of (i) and (ii);

wherein upon introduction of said construct to said animal cell, an RNA transcript resulting from transcription of a gene comprising said endogenous target sequence of nucleotides exhibits an altered capacity for translation into a proteinaceous product and wherein there is substantially no reduction in the level of transcription of said gene comprising the endogenous target sequence and/or total level of RNA transcribed from said gene comprising said endogenous target sequence of nucleotides is not substantially reduced.

Preferably, the animal cell is a mammalian cell such as but not limited to a human or murine animal cell.

The present invention further extends to a genetically modified vertebrate animal cell characterized in that said cell:—

(i) comprises a sense copy of a target endogenous nucleotide sequence introduced into said cell or a parent cell thereof; and

(ii) comprises substantially no proteinaceous product encoded by a gene comprising said endogenous target nucleotide sequence compared to a non-genetically modified form of same cell.

The vertebrate animal cell according to this embodiment is preferably from a mammal, avian species, fish or reptile. More preferably, the animal cell is of mammalian origin such as from a human, primate, livestock animal or laboratory test animal. Particularly preferred animal cells are from human and murine species.

The nucleotide sequence comprising the sense copy of the target endogenous nucleotide sequence may further comprise a nucleotide sequence complementary to said target sequence. Preferably, the identical and complementary sequences are separated by an intron sequence such as, for example, from a gene encoding β-globin (e.g. human β-globin intron 2).

Furthermore, in one embodiment, there is substantially no reduction in levels of steady state total RNA as a result of the introduction of a nucleotide sequence comprising the sense copy of the target sequence.

Accordingly, the present invention provides a genetically modified vertebrate animal cell characterized in that said cell:—

(i) comprises a sense copy of a target endogenous nucleotide sequence introduced into said cell or a parent cell thereof;

(ii) comprises substantially no proteinaceous product encoded by a gene comprising said endogenous target nucleotide sequence compared to a non-genetically modified form of same cell; and

(iii) comprises substantially no reduction in the levels of steady state total RNA relative to a non-genetically modified form of the same cell.

The present invention further extends to transgenic including genetically modified animal cells and cell lines which exhibit a modified phenotype characterized by a post-transcriptionally modulated genetic sequence.

Accordingly, another aspect of the present invention is directed to a animal cell in isolated form or maintained under in vitro culture conditions or an animal comprising said cells wherein the cell or its animal host exhibits at least one altered phenotype compared to the cell or an animal prior to genetic manipulation, said genetic manipulation comprising introducing to an animal cell a genetic construct comprising a nucleotide sequence having substantial homology to a target nucleotide sequence within the genome of said animal cell and wherein the expression of said target nucleotide sequence is modulated at the post-transcriptional level.

Preferably, the nucleotide sequence on the genetic construct is operably linked to a promoter.

Optionally, the genetic construct may comprise two or more nucleotide sequences, each operably linked to one or more promoters and each having homology to an endogenous mammalian nucleotide sequence.

The present invention extends to a genetically modified animal such as a mammal comprising one or more cells in which an endogenous gene is substantially transcribed but not translated resulting in a modifying phenotype relative to the animal or cells of the animal prior to genetic manipulation.

Another aspect of the present invention provides a genetically modified murine animal comprising a nucleotide sequence substantially identical to a target endogenous sequence of nucleotides in the genome of a cell of said murine animal wherein an RNA transcript resulting from transcription of a gene comprising said endogenous target sequence of nucleotides exhibits an altered capacity for translation into a proteinaceous product.

Preferred murine animals are mice and are useful inter alia as experimental animal models to test therapeutic protocols and to screen for therapeutic agents.

In a preferred embodiment, the genetically modified murine animal further comprises a sequence complementary to the target endogenous sequence. Generally, the identical and complementary sequences may be separated by an intron sequence as stated above.

The present invention further contemplates a method of altering the phenotype of a vertebrate animal cell wherein said phenotype is conferred or otherwise facilitated by the expression of an endogenous gene, said method comprising introducing a genetic construct into said cell or a parent of said cell wherein the genetic construct comprises a nucleotide sequence substantially identical to a nucleotide sequence comprising said endogenous gene or part thereof and wherein a transcript exhibits an altered capacity for translation into a proteinaceous product compared to a cell without having had the genetic construct introduced.

Reference herein to homology includes substantial homology and in particular substantial nucleotide similarity and more preferably nucleotide identity.

The term “similarity” as used herein includes exact identity between compared sequences at the nucleotide level. Where there is non-identity at the nucleotide level, “similarity” includes differences between sequences which result in different amino acids that are nevertheless related to each other at the structural, functional, biochemical and/or conformational levels. In a particularly preferred embodiment, nucleotide sequence comparisons are made at the level of identity rather than similarity.

Terms used to describe sequence relationships between two or more polynucleotides include “reference sequence”, “comparison window”, “sequence similarity”, “sequence identity”, “percentage of sequence similarity”, “percentage of sequence identity”, “substantially similar” and “substantial identity”. A “reference sequence” is at least 12 but frequently 15 to 18 and often at least 25 or above, such as 30 monomer units, inclusive of nucleotides, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al. (1997). A detailed discussion of sequence analysis can be found in Unit 19.3 of Ausubel et al. (1998).

The terms “sequence similarity” and “sequence identity” as used herein refer to the extent that sequences are identical or functionally or structurally similar on a nucleotide-by-nucleotide basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) occurs in both sequences to yield the number of matched positions, dividing the number of matched positions by the total number of positions in the window of comparison (i.e. the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity.

The present invention is further directed to the use of genetic construct comprising a sequence of nucleotides substantially identical to a target endogenous sequence of nucleotides in the genome of a vertebrate animal cell in the generation of an animal cell wherein an RNA transcript resulting from transcription of a gene comprising said endogenous target sequence of nucleotides exhibits an altered capacity for translation into a proteinaceous product.

Preferably, the vertebrate animal cell is as defined above and is most preferably a human or murine species.

The construct may further comprise a nucleotide sequence complementary to said target endogenous nucleotide sequence and the nucleotide sequences identical and complementary to said target endogenous nucleotide sequences may be separated by an intron sequence as described above.

In one embodiment, there is no reduction in the level of transcription of said gene comprising the endogenous target sequence and/or steady state levels of total RNA are not substantially reduced.

Still a further aspect of the present invention contemplates a method of genetic therapy in a vertebrate animal, said method comprising introducing into cells of said animal comprising a sequence of nucleotides substantially identical to a target endogenous sequence of nucleotides in the genome of said animal cells such that upon introduction of said nucleotide sequence, RNA transcript resulting from transcription of a gene comprising said endogenous target sequence of nucleotides exhibits an altered capacity for translation into a proteinaceous product.

Reference herein to “genetic therapy” includes gene therapy. The genetic therapy contemplated by the present invention flier includes somatic gene therapy whereby cells are removed, genetically modified and then replaced into an individual.

Preferably, the animal is a human.

The present invention is further described by the following non-limiting Examples.

### EXAMPLE 1

Tissue Culture Manipulations

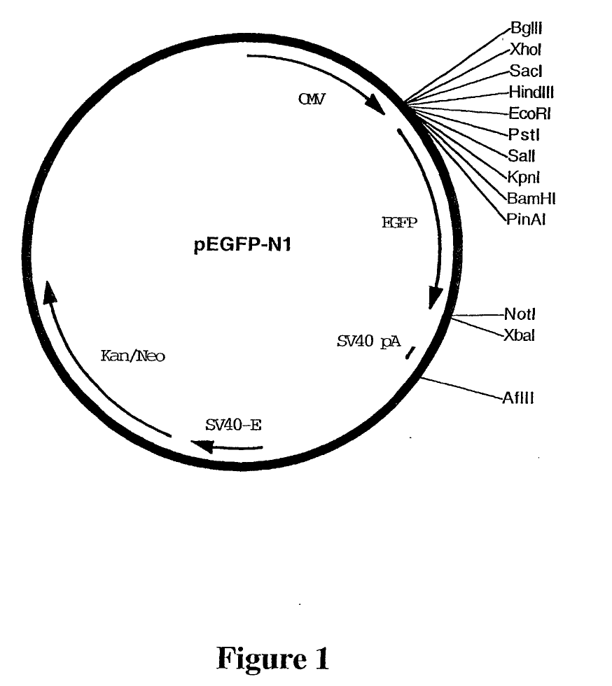

To generate GFP expressing cell lines, PK-1 cells (derived from porcine kidney epithelial cells) were transformed with a construct designed to express GFP, namely pEGFP-N1 (Clontech Catalogue No.: 6085-1; refer to FIG. 1).

PK-1 cells were grown as adherent monolayers using Dulbecco's Modified Eagle's Medium (DMEM; Life Technologies), supplemented with 10% v/v Fetal Bovine Serum (FBS; TRACE Biosciences or Life Technologies). Cells were always grown in incubators at 37° C. in an atmosphere containing 5% vlv CO₂. Cells were grown in a variety of tissue culture vessels, depending on experimental requirements. The vessels used were: 96-well tissue culture plates (vessels containing 96 separate tissue culture wells each about 0.7 cm in diameter, Costar); 48-well tissue culture plates (vessels containing 48 separate tissue culture wells, each about 1.2 cm in diameter; Costar); 6-well tissue culture plates (vessels containing 6 separate wells, each about 3.8 cm diameter, Nunc); or larger T25 and T75 culture flasks (Nunc). For cells transformed with pEGFP-N1, DMEM, 10% (v/v) FBS medium was further supplemented with genetecin (Life Technologies); for initial selection of transformed cells, 1.5 mg/l genetecin was used. For routine maintenance of transformed cells, 1.0 mg/l genetecin was used.

In all instances, medium was changed at 48-72 hr intervals. This was accomplished by removing spent medium, washing the cell monolayers in the tissue culture vessel by adding Phosphate Buffered Saline (1×PBS; Sigma) and gently rocking the culture vessel, removing the 1×PBS and adding fresh medium. The volumes of 1×PBS used in these manipulations were typically 100 μl, 400 μl, 1 ml, 2 ml and 5 ml for 96-well, 48-well, 6-well, T25 and T75 vessels, respectively. Tissue culture media volumes were typically 200 μl for 96-well tissue culture plates, 0.4 ml for 48-well tissue culture plates, 4 ml for 6-well tissue culture plates, 11 ml for T 25 and 40 ml for T75 tissue culture vessels.

During the course of these experiments, it was frequently necessary to change culture vessels. To achieve this, monolayers were washed twice with 1×PBS and then treated with trypsin-EDTA (Life Technologies) for 5 min at 37° C. Under these conditions cells lose adherence and can be resuspended by trituration and transferred to DMEM, 10% v/v FBS, which stops the action of trypsin-EDTA. The volumes of 1×PBS for washing and Trypsin-EDTA used for such manipulations were typically 100 μl, 400 μL, 1 ml, 2 ml and 5 ml for 96-well, 48-well, Swell, T25 and T75 vessels, respectively.

In addition, it was sometimes necessary to count the number of resuspended cells, especially when biologically cloning transformed cell lines. To achieve this, cells were resuspended in an appropriate volume of DMEM, 10% v/v FBS and an aliquot, typically 100 μl, was transferred to a haemocytometer (Hawksley) and cell numbers counted microscopically.

Protocol for Freezing Cells

During the course of the experiments, it was frequently necessary to store PK-1 cell lines for later use. To achieve this, monolayers were washed twice with 1×PBS and then treated with trypsin-EDTA for 5 min at 37° C. The PK-1 cells were resuspended by trituration and transferred to storage medium consisting of DMEM, 20% v/v FBS and 10% v/v dimethylsulfoxide (Sigma). The concentration of PK-1 cells was determined by haemocytometer counting and further diluted to 10⁵ cells per ml. Aliquots of PK-1 cells were transferred to 1.5 ml cryotubes (Nunc). The tubes of PK-1 cells were placed in a Cryo 1° C. Freezing Container (Nalgene) containing propan-2-ol (BDH) and cooled slowly to −70° C. The tubes of PK-1 cells were then stored at −70° C. Reanimation of stored PK-1 cell was achieved by warming the cells to 0° C. on ice. The cells were then transferred to a T25 flask containing DMEM and 20% v/v FBS, and then incubated at 37° C. in an atmosphere of 5% v/v CO₂.

List of Media Components

(a) Dulbecco's Modified Eagle Medium (DMEM)

Two commercial formulations of DMEM were used, both obtained from Life Technologies. The first was a liquid formulation (Cat. no. 11995), the second a powder formulation which was prepared according to the manufacturer's specifications (Cat. no. 23700). Both formulations were used in these experiments and were considered equivalent, despite minor modifications. The liquid formulation (11995) was:—

| | | |
|---|---|---|
| D-glucose | 4,500 | mg/l |
| Phenol Red | 15 | mg/l |
| Sodium pyruvate | 110 | mg/l |
| L-Arginine.HCl | 84 | mg/l |
| L-Cystine.2HCl | 63 | mg/l |
| L-Glutamine | 584 | mg/l |
| Glycine | 30 | mg/l |
| L-Histidine HCl.H₂O | 42 | mg/l |
| L-Isoleucine | 105 | mg/l |
| L-Leucine | 105 | mg/l |
| L-Lysine.HCl | 146 | mg/l |
| L-Methionine | 30 | mg/l |
| L-Phenylalanine | 66 | mg/l |
| L-Serine | 42 | mg/l |
| L-Threonine | 95 | mg/l |
| L-Tryptophan | 16 | mg/l |
| L-Tyrosine.2Na.2H₂O | 104 | mg/l |
| L-Valine | 94 | mg/l |
| CaCl₂ | 200 | mg/l |
| Fe(NO₃)₃.9H₂O | 0.1 | mg/l |
| KCl | 400 | mg/l |
| MgSO₄ | 97.67 | mg/l |
| NaCl | 6,400 | mg/l |
| NaHCO₃ | 3,700 | mg/l |
| NaH₂PO₄.H₂O | 125 | mg/l |
| D-Ca pantothenate | 4 | mg/l |
| Choline chloride | 4 | mg/l |
| Folic Acid | 4 | mg/l |
| i-Inositol | 7.2 | mg/l |
| Niacinamide | 4 | mg/l |
| Riboflavin | 0.4 | mg/l |
| Thiamine HCl | 4 | mg/l |
| Pyridoxine HCl | 4 | mg/l |

When reconstituted the powdered formulation (23700) was identical to the above, except it contained HEPES at 4,750 mg; sodium pyruvate and NaHCO₃ were omitted and NaCl was used at 4,750 mg/l, not 6,400 mg/l.

(b) OP7T-MEM I (Registered Trademark) Reduced Serum Medium

This is a commercial modification of MEM (Life Technologies Cat. No. 31985), designed to permit growth of cells in serum free medium. Such serum free media are commonly used in experiments where cationic lipid transfectants such as GenePORTER2 (trademark) or LIPOFECTAMINE (trademark) are used, since higher transfection frequencies are obtained.

(c) Phosphate Buffered Saline (PBS)

Phosphate buffered saline was prepared from a commercial powder mix (Sigma, Cat. No. P-3813) according to manufacturer's instructions. A 1×PBS solution (pH 7.4) consists of:

| | | |
|---|---|---|
| Na₂HPO₄ | 10 | mM |
| KH₂PO₄ | 1.8 | mM |
| NaCl | 138 | mM |
| KCl | 2.7 | mM |

(d) Trypsin-EDTA

Trypsin-EDTA is commonly used to loosen adherent cells to permit their passage. In these experiments a commercial preparation (Life Technologies, Cat. No. 15400) was used. This is a 10×stock solution consisting of:

| | | |
|---|---|---|
| Trypsin | 5 | g/l |
| EDTA.4Na | 2 | g/l |
| NaCl | 8.5 | g/l |

To prepare working stocks, this solution was diluted using 9 volumes of 1×PBS.

### EXAMPLE 2

Generating Stable EGFP-Transformed Cell Lines

Transformations were performed in 6-well tissue culture vessels. Individual wells were seeded with 1×10³ PK-1 cells in 2 ml of DMEM, 10% v/v EBS, and incubated until the monolayer was 60-90% confluent, typically 24 to 48 hr.

To transform a single plate (6 wells), 12 μg of plasmid pEGFP-N1 and 108 μl of GenePORTER2 (trademark) (Gene Therapy Systems) were diluted into OPTI-MEM I (registered trademark) medium to obtain a final volume of 6 ml and incubated at room temperature for 45 min.

The tissue growth medium was removed from each well and each well was washed with 1 ml of 1×PBS as described above. The monolayers were overlayed with 1 ml of the plasmid DNA/GenePORTER conjugate for each well and incubated at 37° C., 5% v/v CO₂ for 4.5 hr.

One ml of OPTI-MEM I (registered trademark) supplemented with 20% v/v FBS was added to each well and the vessel incubated for a further 24 hr, at which time cells were washed with 1×PBS and medium was replaced with 2 ml of fresh DMEM including 10% v/v FBS. At this stage, monolayers were inspected for transient GFP expression using fluorescence microscopy.

Forty-eight hr after transfection the medium was removed, cells washed with PBS as above and 4 ml of fresh DM1AM containing 10% v/v FBS supplemented with 1.5 mg/l genetecin was added to each well; genetecin was included in the medium to select for stably transformed cell lines. The DMEM, 10% v/v FBS, 1.5 mg/l genetecin medium was changed every 48-72 hr. After 21 days of selection, putatively transformed colonies were apparent. At this stage, cells were transferred to larger culture vessels for expansion, maintenance and biological cloning.

To remove transformed colonies, cells were treated with trypsin-EDTA as described above in Example 1 and transferred to 11 ml of DMEM, 10% v/v FBS, 1.5 mg/l genctecin and incubated in a T25 culture vessel at 37° C. and 5% v/v CO₂. When these monolayers were about 90% confluent, cells were resuspended using Trypsin-EDTA, then transferred to 40 ml DMEM, 10% v/v FBS, 1.5 mg/l geneticin. Vessels were incubated at 37° C. and 5% v/v CO₂. When monolayers became confluent, they were passaged every 48-72 hr by trypsin-treating cells as above and diluting one tenth of the cells into 40 ml fresh DMEM, 10% v/v FBS, 1.5 mg/l genetecin. At this point, some cells were also frozen for long term maintenance. These cultures contained mixtures of transformed cell lines.

### EXAMPLE 3

Dilution Cloning of Transformed Cell Lines

Transformed cells were biologically cloned using a dilution strategy, whereby colonies were established from single cells. To support growth of single colonies, “conditioned media” were used. Conditioned media were prepared by overlaying 20-30% confluent monolayers of PK-1 cells grown in a T75 vessel with 40 ml of DMEM containing 10% v/v FBS. Vessels were incubated at 37° C., 5% v/v CO₂ for 24 hr, after which the growth medium was transferred to a sterile 50 ml tube (Falcon) and centrifuged at 500×g. The growth medium was passed through a 0.45 μm filter and decanted to a fresh sterile tube and used as “conditioned medium”.

A T75 vessel containing mixed colonies of transformed PK-1 cells at 20-30% confluency was washed twice with 1×PBS and cells separated by trypsin treatment as described above, then diluted into 10 ml of DMEM, 10% v/v FBS. The cell concentration was determined microscopically using a haemocytometer slide and cells diluted to 10 cells per ml in conditioned medium. Single wells of 96-well tissue culture vessels were seeded with 200 μl of the diluted cells in conditioned medium and cells were incubated at 37° C. and 5% v/v CO₂ for 48 hr. Wells were inspected microscopically and those containing a single colony, arising from a single cell, were defined as clonal cell lines. The original conditioned medium was removed and replaced with 200 μl of fresh conditioned medium and cells incubated at 37° C. and 5% v/v CO₂ for 48 hr. After this time, conditioned medium was replaced with 200 μl of DMEM, 10% v/v FBS and 1.5 mg/l genetecin and cells again incubated at 37° C. and 5% v/v CO₂. Colonies were allowed to expand and medium was changed every 48 hr.

When the monolayer in an individual well was about 90% confluent, the cells were washed twice with 100 μl of 1×PBS and cells loosened by treatment with 20 μl of 1×PBS/1×trypsin-EDTA as described above. Cells in a single well were transferred to a single well of a 48-well culture vessel containing 500 μl of DMEM, 10% v/v FBS and 1.5 μg/ml genetecin. Medium was changed every 48-72 hr as hereinbefore described.

When a monolayer in an individual well of a 48-well culture vessel was about 90% confluent, the cells were transferred to 6-well tissue culture vessels using trypsin-EDTA treatment as described above. Separated cells were then transferred to 4 ml DEM, 10% v/v FBS, 1.5 μg/ml geneticin and transferred to a single well of a 6-well tissue culture vessel. Cells were grown at 37° C. and 5% v/v CO₂ and colonies were allowed to expand. Medium was changed every 48 hr.

When monolayers in 6-well culture vessels were about 90% confluent, cells were transferred to T25 vessels using trypsin-EDTA as described above. When these monolayers were about 90% confluent, cells were transferred to T75 culture vessels, as described above. Once individual lines were established in T75 vessels they were either maintained by passaging every 48-72 hr using a one-tenth dilution, or maintained as frozen stocks.

### EXAMPLE 4

Preparation of Nuclei for Transcription Run-On Assays

To analyze the status of transcription of individual genes in cloned transformed cell lines, nuclear run-on assays were performed. A monolayer of cells was established by seeding a T75 culture vessel with 4×10⁶ transformed PK-1 cells into 40 ml of DMEM, 10% v/v FBS and incubating cells until the monolayer was about 90% confluent. The monolayers were washed twice with 5 ml of 1×PBS, separated by treatment with 2 ml trypsin-EDTA and transferred to 2 ml of DMEM including 10% v/v FBS.

These cells were transferred to a 10 ml capped tube, 3 ml of ice-cold 1×PBS was added and the contents mixed by inversion. Transformed PK-1 cells were collected by centrifugation at 500×g for 10 min at 4° C., the supernatant was discarded and cells were resuspended in 3 ml of ice-cold 1×PBS by gentle vortexing. Total cell numbers were determined using a haemocytometer, a maximum of 2×10⁸ cells was used for subsequent analyses.

Transformed PK-1 cells were collected by centrifugation at 500×g for 10 min at 4° C. and resuspended in 4 ml Sucrose buffer 1 (0.3 M sucrose, 3 mM calcium chloride, 2 mM magnesium acetate, 0.1 mM EDTA, 10 mM Tris-HCl (pH 8.0), 1 mM dithiothreitol (DTT), 0.5% v/v Igepal CA-630 (Sigma)). Cells were incubated at 4° C. for 5 min to allow them to lyse then small aliquots were examined by phase-contrast microscopy. Under these conditions lysis can be visualized. Homogenates were transferred to 50 ml tubes containing 4 ml of ice-cold Sucrose buffer 2 (1.8 M sucrose, S mM magnesium acetate, 0.1 mM EDTA, 10 mM Tris-HCl (pH 8.0), 1 mM DTT).

To obtain efficient transcription run-on assays, nuclei should be purified from other cellular debris. One method for this is to purify nuclei by ultra-centrifugation through sucrose pads. The final concentration of sucrose in a cell homogenate should be sufficient to prevent a large build up of debris at the interface between homogenate and the sucrose cushion. Therefore, the amount of Sucrose buffer 2 added to the initial cell homogenate was varied in some instances.

To prepare a sucrose pad, 4.4 ml ice-cold Sucrose buffer 2 was transferred to a polyallomer SW41 tube (Beckman). Nuclear preparations were carefully layered over the sucrose pad and centrifuged for 45 min at 30,000×g (13,300 rpm in SW41 rotor) at 4° C. The supernatant was removed and the pelleted nuclei loosened by gentle vortexing for 5 seconds. Nuclei were resuspended by trituration in 200 μl ice cold glycerol storage buffer (50 mM Tris-HCl (pH 8.3), 40% v/v glycerol, 5 mM magnesium chloride, 0.1 mM EDTA) per 5×10⁷ nuclei. One hundred microlitres of this suspension (approximately 2.5×10⁷ nuclei) was aliquoted into chilled microcentrifuge tubes and 1 μl (40 U) RNasin (Promega) was added. Usually such extracts were used immediately for transcription run-on assays, although they could be frozen on dry ice and stored at −70° C. or in liquid nitrogen for later use.

### EXAMPLE 5

Nuclear Transcription Run-On Assays

All NTPs were obtained from Roche. Nuclear run-on reactions were initiated by adding 100 μl of 1 mM ATP, 1 mM CTP, 1 mM GTP, 5 mM DTT and 5 μl (50 μCi) [α³²P]-UTP (GeneWorks) to 100 μl of isolated nuclei, prepared as hereinbefore described. The reaction mix was incubated at 30° C. for 30 min with shaking and terminated by adding 400 μl of 4 M guanidine thiocyanate, 25 mM sodium citrate (pH 7.0), 100 mM 2-mercaptoethanol and 0.5% v/v N-lauryl sarcosine (Solution D). To purify in vitro synthesized RNAs, 60 μl 2 M sodium acetate (pH 4.0) and 600 μl water-saturated phenol was added and the mixture vortexed; an additional 120 μl chloroform/isoamylalcohol (49:1) was added, the mixture vortexed and phases separated by centrifugation.

The aqueous phase was decanted to a fresh tube and 20 μg tRNA added as a carrier. RNA was precipitated by the addition of 650 μl isopropanol and incubation at −20° C. for 10 min. RNA was collected by centrifugation at 12,000 rpm at 4° C. for 20 min and the pellet was rinsed with cold 70% v/v ethanol. The pellet was dissolved in 30 μl of TE pH 7.3 (10 mM Tris-HCl, 1 mM EDTA) and vortexed to resuspend the pellet. 400 μl of Solution D was added and the mixture vortexed. The RNA was precipitated by the addition of 430 μl of isopropanol, incubation at −20° C. for 10 mins and centrifuged at 10,000 g for 20 mins at 4° C. The supernatant was removed and the RNA pellet washed with 70% v/v ethanol. The pellet was resuspended in 200 μl of 10 mM Tris (H 7.3), 1 mM EDTA and incorporation estimated with a hand-held geiger counter.

To prepare the radioactive RNAs for hybridization, samples were precipitated by adding 20 μl 3 M sodium acetate pH 5.2, 500 μl ethanol and collected by centrifugation at 12,000×g and 4° C. for 20 min. The supernatant was removed and the pellet resuspended in 1.5 ml of hybridization buffer (MRC #HS 114F, Molecular Research Centre Inc.).

### EXAMPLE 6

Dot Blot Filter Preparation

Dot blot filters were prepared for the detection of ³²P-labelled nascent mRNA transcripts prepared as hereinbefore described. A Hybond NX filter (Amersham) was prepared for each PK-1 cell line analyzed. Each filter that was prepared contained four plasmids at four successive one-fifth dilutions. The plasmids were pBluescript (registered trademark) II SK⁺ (Stratagene), pGEM.Actin (Department of Microbiology and Parasitology, University of Queensland), pCMV.Galt, and pBluescriptEGFP.

The plasmid pCMV.Galt was constructed by replacing the EGFP open reading frame of pEGFP-N1 (Clontech) with the porcine α-1,3-galactosyltransferase (GalT) structural gene sequence. Plasmid pEGFP-N1 was digested with PinAI and Not I, blunted-ended using PfuI polymerase and then re-ligated creating the plasmid pCMV.cass. The GalT structural gene was excised from pCDNA3.GalT (B3resagen) as an EcoRI fragment and ligated into the EcoRI site of pCMV.cass.

The plasmid pBluescript.EGFP was constructed by excising the EGFP open reading frame of pEGFP-N1 and ligating this fragment into the plasmid pBluescript (registered trademark) II SK⁺. Plasmid pEGFP-N1 was digested with NotI and XhoI and the fragment NotI-EGFP-Xho was then ligated into the NotI and XhoI sites of pBluescript II SK⁺.

Ten micrograms of plasmid DNA for each construct was digested in a volume of 200 μl with the EcoRI. The mixture was extracted with phenol/chloroform/isoamylalcohol followed by chloroform/isoamylalcohol extracted, then ethanol precipated. The plasmid DNA pellet was suspended in 500 μl of 6×SSC (0.9 M Sodium Chloride, 90 mM Sodium Citrate; pH 7.0) and then diluted in 6×SSC at concentrations of 1 μg/50 μl, 200 ng/50 μl, 40 ng/50 μl and 8 ng/50 μl. The plasmids was heated to 100° C. for 10 min and then cooled on ice.

An 8×11.5 cm piece of Hybond NX filter was soaked in 6×SSC for 30 min The filter was then placed into a 96-well (3 mm) dot-blot apparatus (Life Technologies) and vacuum locked. Five hundred microlitres of 6×SSC was loaded per slot and the vacuum applied. While maintaining the vacuum, 50 μl of each plasmid DNA concentration for each plasmid was loaded onto the filter as a 4×4 matrix. This was replicated six times across the filter. While maintaining the vacuum, 250 μl of 6×SSC was loaded per slot. The vacuum was then released. The filter was placed (DNA side up) for 10 min on blotting paper soaked in denaturing solution (1.5 M Sodium Chloride, 0.5 M Sodium Hydroxide). The filter was then transferred to blotting paper soaked in neutralising solution and soaked for 5 min in 1 M sodium chloride, 0.5 M Tris-HCl (pH 7.0).

The filter was placed in a GS Gene Linker (Bio Rad) and 150 mJoules of energy applied to cross-link the plasmid DNA to the filter. The filter was rinsed in sterile water. To check the success of the blotting procedure, the filter was stained with 0.4% v/v methylene blue in 300 mM sodium acetate (pH 5.2) for 5 min. The filter was rinsed twice in sterile water and then de-stained in 40% v/v ethanol. The filter was then rinsed in sterile water to remove the ethanol and cut into its six individual replicates of the four-plasmid/concentration matrix.

### EXAMPLE 7

Filter Hybridization of Nuclear Transcripts

Dot blot or Southern blot filters were transferred to a 10 ml MacCartney bottle and 2 ml of prehybridization solution (Molecular Research Centre Inc. # WP 117) added to each bottle. Filters were incubated at 42° C. overnight in an incubation oven with slow rotation (Hybaid).

The prehybridization buffer was removed and replaced with 1.5 ml of hybridization buffer (MRC #HS 114F, Molecular Research Centre Inc.) containing ³²P-labelled nascent RNA, as described in Examples 5 and 6, and this probe was hybridized to the filters at 42° C. for 48 hr.

Following hybridization, the radioactively-labelled hybridization buffer was removed and the filters washed in washing solution (MRC #WP 117). Filters were washed in a total of 5 changes of wash solution, each change being 2 ml. The washes were performed in the hybridization oven; the first three washes were at 30° C., the last two washes at 50° C.

To further increase stringency and reduce background, filters were treated with RNase A. Filters were placed into 5 ml 10 μg/ml RNase A (Sigma), 10 mM Tris (pH 7.5), 50 mM NaCl and incubated at 37° C. for 5 min.

Filters were then wrapped in plastic wrap and exposed to X-ray film

### EXAMPLE 8

Co-Suppression in Mammalian Cells: EGFP

Six PK-1 cell lines have thus far been examined. These six lines consist of one untransformed control line (wild type) and five lines transformed with the construct pCMV.EGFP (refer to Example 1). Two of these five lines are positive for EGFP expression as visualized by microscopic examination under UV light. All cells of the monolayer from line A4g are EGFP positive, while approximately 0.1% of the monolayer cells for line A7g are EGFP positive. The remaining lines C3, C8, and C10 are visually negative for EGFP expression.

Nuclear transcription run-on assays were performed as described in Examples 4 to 7, above. In filter hybridization analysis of the labelled products the inclusion of the four plasmids at four concentrations serves two purposes. The four concentrations specifically indicate the minimum concentration of target plasmid required to detect the target mRNA transcript. The four plasmids serve as specific targets and controls for the experiment The plasmids serve the following functions.

pBluescript II SK⁺

This plasmid is to check for non-specific hybridization of synthesized nuclear RNA to the plasmid backbone common to all the target constructs used.

pBluescript.EGFP

This plasmid is the target of ³²P-labelled nuclear EGFP RNA. Hybridization to this plasmid indicates active transcription of EGFP RNA This was evident in lines A4g, A7g, C3 and C8, but not evident in line C10.

pCMV.GalT

GalT (α-1,3-galactosidyl transferase) is an endogenous porcine gene. This plasmid thus serves as a positive control target for an endogenous porcine gene.

pGem.Adin

β-actin is a ubiquitous gene of eukaryotes and a common mRNA species. This plasmid, containing a chicken β-actin cDNA sequence, serves as an additional positive control.

The following conclusions can be drawn from the results of these experiments:

(1) Non-specific hybridization to the plasmid backbone of these constructs did not occur. Hybridization to the GalT positive control did not occur for all lines, in agreement with expectation since the mRNA of this gene is not abundant.

(2) Hybridization to the β-actin gene positive control occurred for all lines in agreement with expectation, given the mRNA of this gene is abundant.

(3) Hybridization to the EGFP gene by nascent RNA for the lines A4g and A7g was as expected based on visual observations of EGFP expression in these lines.

(4) Hybridization to the EGFP gene by nascent RNA for silenced lines C3 and C8 is indicative of co-suppression of EGFP transcripts under normal growth conditions for these lines.

(5) Co-suppression activity in line C10 has not been demonstrated in this experiment.

Table 1 summarizes the expected outcome and the observed outcomes for the hybridization of ³²P-labelled nuclear RNA to the aforementioned plasmids. Table 1 also indicates the minimum concentration of target plasmid DNA for which hybridization of the specific nuclear RNA was onserved.

TABLE 1

| | | | | pBluescriptII | | | | pBluescriptII | | | |
|---|---|---|---|---|---|---|---|---|---|---|---|
| Cell | EGFP | Target | | SK⁺ | | pCMV.GalT | | EGFP | | pGem.Actin | |
| Line | Express | Amount | | Exp | Obs | Exp | Obs | Exp | Obs | Exp | Obs |
| PK | No | | | Nil | Nil | Hyb'n | Hyb'n | Nil | Nil | Hyb'n | Hyb'n |
| A4g | Yes | 1 | μg | Nil | Nil | Hyb'n | Hyb'n | Hyb'n | Hyb'n | Hyb'n | Hyb'n |
| A7g | Yes | 1 | μg | Nil | Nil | Hyb'n | Hyb'n | Hyb'n | Hyb'n | Hyb'n | Hyb'n |
| C3 | No | >200 | ng | Nil | Nil | Hyb'n | Hyb'n | Hyb'n | Hyb'n | Hyb'n | Hyb'n |
| C8 | No | 1 | μg | Nil | Nil | Hyb'n | Nil | Hyb'n | Hyb'n | Hyb'n | Hyb'n |
| C10 | No | 1 | μg | Nil | Nil | Hyb'n | Nil | Hyb'n | Nil | Hyb'n | Hyb'n |

EGFP Express—EGFP Expression
Exp = Expected result for PTGS
Obs = Observed result
Hyb'n = hybridization

### EXAMPLE 9

Co-Suppression of Genes

The inventors demonstrate co-suppression of a transgene, enhanced green fluorescent protein EGFP), in cultured porcine kidney cells. The inventors further demonstrate co-suppression of a broad range of endogenous genes in different cell types and agents such as viruses, cancers and transplantation antigen. Particular targets include:

(a) Bovine enterovirus (BEV). Frozen lines of BEV-transformed cells are revived and grown through many generations over several weeks/months before being challenged with BEV. Cells that are effectively co-suppressed are not killed by the virus immediately. This viral-tolerant phenotype provides a demonstration of utility.

(b) Tyrosinase, the product of a gene essential for melanin (black) pigment formation in skin. Silencing of the tyrosinase gene is readily detected in cultured mouse melanocytes and subsequently in black strains of mice.

(c) Galactosyl transferase (GalT). Silencing of the GalT gene occurs in parallel with cell death although GalT itself is not essential to cell survival. The inventors assume that cell death occurs because GalT is one member of a gene family, where members of the family share a similar DNA sequence(s), reflecting similarity of function (transfer of sugar residues). Some of these genes may be essential to cell survival. The inventors transform pig cells with 3′ untranslated region (3′-UTR) of the GalT gene, rather than the entire gene, to target segments that are unique to GalT for degradation, and hence silence GalT alone.

(d) Thymidine kinase (TK) converts thymidine to thymidine monophosphate (TMP). The drug 5-bromo-2′-deoxyuridine (BrdU) selects cells that have lost TK. In cells with functioning TK, the enzyme converts the drug analogue to its corresponding 5′-monophosphate, which is lethal once it is incorporated into DNA. NIH/3T3 cells are transformed with a construct comprising the TK gene. Cells that are effectively co-suppressed will tolerate the addition of BrdU to the growth medium and will continue to replicate.

(e) A cellular oncogene such as HER-2 or Brn-2, associated with transformation of normal cells into cancer cells.

(f) A cell surface antigen on a human and/or mouse haemopoietic (“blood-forming”) cell line. These cells are the precursors of white blood cells, responsible for immunity; they are characterized by specific surface antigens which are essential to their immune function. A particular advantage of this system is that the cells grow in suspension (rather than being attached to the culture vessel and to each other) so are easily examined by microscope and quantified by fluorescence activated cell sorting (FACS). In addition, a vast range of reagents is available for identifying specific antigens.

(g) Tyrosinase, the product essential for melanin (black) pigment production in melanocytes in mice. In transgenic mice, inactivation of the endogenous tyrosinase can be readily detected as a change in coat colour of animals in strains that normally produce melanin. Such a phenotype provides demonstration of utility in transgenic animals.

(h) Galactosyl transferase (GalT) catalyses the addition of galactosyl residues to cell surface proteins. Inactivation of GalT in transgenic mice can be readily detected by assaying tissues of transgenic animals for loss of galactosyl residues and provides demonstration of utility in transgenic animals.

(i) YB-1 (Y-box DNA/RNA-binding factor 1) is a transcription factor that binds, inter alia, to the promoter region of the p53 gene and in so doing represses its expression. In cancer cells that express normal p53 protein at normal levels (some 50% of all human cancers), the expression of p53 is under the control of YB-1, such that silencing of YB-1 results in increased levels of p53 protein and consequent apoptosis.

### EXAMPLE 10

Generic Techniques

1. Tissue Culture Manipulations

(a) Adherent Cell Lines

Adherent cell monolayers were grown, maintained and counted as described in Example 1. Growth medium consisted of either DMEM supplemented with 10% v/v FBS or RPMI 1640 Medium (Life Technologies) supplemented with 10% v/v FBS. Cells were always grown in incubators at 37° C. in an atmosphere containing 5% v/v CO₂.

During the course of these experiments it was frequently necessary to passage the cell monolayer. To achieve this, the monolayers were washed twice with 1×PBS and then treated with trypsin-EDTA for 5 min at 37° C. The volumes of trypsin-EDTA used for such manipulations were typically 20 μl, 100 μl, 500 μl, 1 ml and 2 ml for 96 well, 48 well, 6 well, T25 and 175 vessels, respectively. The action of the trypsin-EDTA was stopped with an equal volume of growth medium. The cells were suspended by trituration. A ⅕ volume of the cell suspension was then transferred to a new vessel containing growth medium. Tissue culture medium volumes were typically 192 μl for 96-well tissue culture plates, 360 μl for 48-well tissue culture plates, 3.8 ml for 6-well tissue culture plates, 9.6 ml for T25 and 39.2 ml for T75 tissue culture vessels.

Cell suspensions were counted as described in Example 1, above.

(b) Non-Adherent Cells

Non-adherent cells were grown in growth medium similarly to adherent cell lines.

As in the case of adherent monolayers, frequent changes of tissue culture vessels were necessary. For T25 and T75 vessels, the cell suspension was removed to 50 ml sterile plastic tubes (Falcon) and centrifuged for 5 min at 500×g and 4° C. The supernatant was then discarded and the cell pellet suspended in growth medium. The cell suspension was then placed into a new tissue culture vessel. For 96-well, 48-well, and 6-well vessels, the vessels were centrifuged for 5 min at 500×g and 4° C. The supernatant was then aspirated away from the cell pellet and the cells suspended in growth medium. The cells were then transferred to a new tissue culture vessel. Tissue culture media volumes were typically 200 μl for 96-well tissue culture plates, 400 μl for 48-well tissue culture plates, 4 ml for 6-well tissue culture plates, 11 ml for T25 and 40 ml for T75 tissue culture vessels.

Passaging the cell suspensions was achieved in the following manner. Cells were centrifuged for 5 min at 500×g and 4° C. and suspended in 5 ml growth medium. Then 0.5 ml (T25) or 1.0 ml (T75) of the cell suspension was transferred to a new vessel containing growth medium. For cells in 96-well, 48-well, and 6-well plates, a ⅕ volume of cells was transferred to the corresponding wells of a new vessel containing ⅘ volume of growth medium.

Cells were counted as described for adherent cells.

2. Protocol for Freezing Cells

Cells stored for later use were frozen according to the protocol outlined in Example 1, above. Adherent monolayers were washed twice with 1×PBS and then treated with trypsin-EDTA (Life Technologies) for 5 min at 37° C. Non-adherent cells were centrifuged for 5 min at 500×g and 4° C. The cells were suspended by trituration and transferred to storage medium consisting of DMEM RPMI 1640 supplemented with 20% v/v FBS and 10% v/v dimethylsulfoxide (Sigma).

3. Cloning of Cell Lines

Adherent and non-adherent mammalian cell types were transfected with specific plasmid vectors carrying expression constructs to target specific genes of interest. Stable, transformed cell colonies were selected over a period of 2-3 weeks using cell growth medium (either DMEM, 10% v/v FBS or RPMI 1640, 10% v/v FBS) supplemented with geneticin or puromycin. Individual colonies were cloned to establish new transfected cell lines.

(a) Adherent Cells

As opposed to the dilution cloning method outlined in Example 3, above, in further examples using adherent cells, individual lines were cloned from discrete colonies as follows. First, the medium was removed from an individual well of a 6-well tissue culture vessel and the cell colonies washed twice with 2 ml of 1×PBS. Next, individual colonies were detached from the plastic culture vessel with a sterile plastic pipette tip and moved to a 96-well plate containing 200 μl of conditioned medium (see Example 1) supplemented with either geneticin or puromycin. The vessel was then incubated at 37° C. and 5% v/v CO₂ for approximately 72 hr. Individual wells were microscopically examined for growing colonies and the medium replaced with fresh growth medium. When the monolayer of each stable line had reached about 90% confluency it was transferred in successive steps as previously described until the stable, transformed line was housed in a T25 tissue culture vessel. At this point, aliquots of each stable cell line were frozen for long term maintenance.

(b) Non-Adherent Cells

Non-adherent cells were cloned by the dilution cloning method described in Example 3.

4. Cell Nuclei Isolation Protocol

(a) Adherent Cells

A 100 mm Petri dish (Costar) or T75 flask containing 30 ml of growth medium (DMEM or RPMI 1640) including 10% v/v FBS was seeded with 4×10⁶ cells and incubated at 37° C. and 5% v/v CO₂ until the monolayer was about 90% confluent (overnight). The Petri dish containing the monolayer was placed on a bed of ice and chilled before processing. Medium was decanted and 8 ml of 1×PBS (ice cold) was added to the Petri dish, and the tissue monolayer washed by gently rocking the dish. The PBS was again decanted and the wash repeated.

The tissue monolayer was overlaid with 4 ml of ice-cold sucrose buffer A [0.32 M sucrose; 0.1 mM EDTA; 0.1% v/v Igepal; 1.0 mM DTT; 10 mM Tris-HCl, pH 8.0; 0.1 mM PMSF; 1.0 mM EGTA; 1.0 mM Spermidine] and cells lysed by incubating them on ice for 2 min. Using a cell scraper, adherent cells were dislodged and a small aliquot of cells examined by phase-contrast microscopy. If the cells had not lysed, they were transferred to an ice-cold dounce homogenizer (Braun) and broken with 5-10 strokes of a type S pestle. Additional strokes were sometimes required. Cells were then examined microscopically to verify that nuclei were free from cytoplasmic debris. Ice-cold sucrose buffer B [1.7 M sucrose; 5.0 mM magnesium acetate; 0.1 mM EDTA; 1.0 mM DTT. 10 mM Tris-HCl, pH 8.0; 0.1 mM PMSF] (4 ml) was then added to the Petri dish and the buffers mixed by gentle stirring with the cell scraper. The final concentration of sucrose in cell homogenates should be sufficient to prevent a large build-up of debris at the interface between the homogenate and the sucrose cushion. The amount of sucrose buffer 2 added to cell homogenate may need to be adjusted accordingly.

(b) Non-Adherent Cells

A T75 tissue culture vessel containing 30 ml of growth medium (D[MEM or RPMI 1640) including 10% v/v FBS was seeded with 4×10⁶ cells and incubated at 37° C. and 5% v/v CO₂ overnight.

The contents of the T75 flask were transferred to a 50 ml screw-capped tube (Falcon), which was placed on ice and allowed to chill before processing. The tube was centrifuged at 500×g for 5 min in a chilled centrifuge to pellet cells. Medium was decanted, 10 ml of 1×PBS (ice cold) added to the tube and the cells suspended by gentle trituration. The PBS was again decanted and the wash repeated.

Cells were suspended in 4 ml of ice-cold sucrose buffer A and lysed by incubating on ice for 2 min and, optionally, by dounce homogenisation, as described above for adherent cells lines.

(c) Isolation Protocol

Nuclei were isolated from cellular debris by sucrose pad centrifugation, according to the protocol described in Example 4, except that sucrose buffers 1 and 2 were replaced by sucrose buffers A and B, respectively.

5. Nuclear Transcription Run-On Protocol

Example 5 provides the method, by nuclear transcription run-on protocol, for the preparation of [α-³²P]-UTP-labelled nascent RNA transcripts for gene-specific detection by filter hybridization (Examples 6, 7 and 8). To detect gene-specific transcription run-on products, an alternative approach to filter hybridization is the ribonuclease protection assay. Strand-specific, gene-specific unlabelled RNA probes are prepared using standard techniques. These are annealed to ³²P-labelled RNAs isolated from transcription run-on experiments. To detect double-stranded RNA, annealing reaction products are treated with a mixture of single strand specific RNases and reaction products are examined using PAGE. Techniques for this are well known to those experienced in the art and are described in RPA III (trademark) handbook ‘Ribonuclease Protection Assay’ (Catalog #s 1414, 1415, Ambion Inc.).

An additional method was used for the preparation of biotin-labelled nascent RNA transcripts (Patrone et al., 2000) for gene specific detection by real-time PCR assays. Intact nuclei were isolated from adherent and non-adherent cell types (refer to Examples 12-19, below) and stored at −70° C. in concentrations of 1×10⁸ per ml in glycerol storage buffer [50 mM Tris-HCl, pH 8.3; 40% v/v glycerol, 5 mM MgCl₂ and 0.1 mM EDTA].

One hundred microlitres of nuclei (10⁷) in glycerol storage buffer was added to 100 μl of ice cold reaction buffer supplemented with nucleotides [200 mM KCl, 20 mM Tris-HCl pH 8.0, 5 mM MgCl₂, 4 mM dithiothreitol (DTT), 4 mM each of ATP, GTP and CTP, 200 mM sucrose and 20% v/v glycerol]. Biotin-16-UTP (from 10 mM tetralithium salt; Sigma) was supplied to the mixture, which was incubated for 30 min at 29° C. The reaction was stopped, the nuclei lysed and digestion of DNA initiated by the addition of 20 μl of 20 mM calcium chloride (Sigma) and 10 μl of 10 mg/ml RNase-free DNase I (Roche). The mixture was incubated for 10 min at 29° C.

Isolation of nuclear run-on and total, including cytoplasmic, RNA was performed using TRIzol (registered trademark) reagent (Life Technologies) as per the manufacturer's instructions. RNA was suspended in 50 μl of RNase-free water. Nascent biotin-16-UTP-labelled run-on transcripts are then purified from total RNA using streptavidin beads Dynabeads (registered trademark) kilobaseBINDER (trademark) Kit, Dynal) according to the manufacturer's instructions.

Real-time PCR reactions are performed to quantify gene transcription rates from these run-on experiments. Real-time PCR chemistries are known to those familiar with the art. Sets of oligonucleotide primers are designed which are specific for transgenes, endogenous genes and ubiquitously-expressed control sequences. Oligonucleotide amplification and reporter primers are designed using Primer Express software (Perkin Elmer). Relative transcript levels are quantified using a Rotor-Gene RG-2000 system (Corbett Research).

6. Detection of mRNA

Ribonuclease protection assay, using the method of annealing unlabelled mRNA to 32P-labelled probes, may be used to detect transcripts of endogenous genes and transgenes in the cytoplasm. Reaction products are examined using PAGE. Steady state levels of RNA products of endogenous genes and transgenes are assessed by Northern analysis.

Alternatively, relative mRNA levels are quantified using real-time PCR with a Rotor-Gene RG-2000 system with amplification and reporter oligonucleotides designed using Primer Express software for specific transgenes, endogenous genes and ubiquitously-expressed control genes.

7. Southern Blot Analysis of Mammalian Genomic DNA

For all subsequent examples, Southern blot analyses of genomic DNA were carried out according to the following protocol. A T75 tissue culture vessel containing 40 ml of DMEM or RPMI 1640, 10% v/v FBS was seeded with 4×10⁶ cells and incubated at 37° C. and 5% v/v CO₂ for 24 hr.

(a) Adherent Cells

For adherent cells, proceed as follows: decant medium and add 5 ml of 1×PBS to the T75 flask and wash the tissue monolayer by gently rocking. Decant the PBS and repeat washing of the tissue monolayer with 1×PBS. Decant the PBS. Overlay the monolayer with 2 ml 1×PBS/1×Trypsin-EDTA. Cover the surface of the tissue monolayer evenly by gentle rocking of the flask. Incubate the T75 flask at 37° C. and 5% v/v CO₂ until the tissue monolayer separates from the flask Add 2 ml of medium including 10% v/v FBS to the flask. Under microscopic examination, the cells should now be single and round. Transfer the cells to a 10 ml capped tube and add 3 ml of ice-cold 1×PBS. Invert the tube several times to mix. Pellet the cells by centrifugation at 500×g for 10 min in a refrigerated centrifuge (4° C.). Decant the supernatant and add 5 ml of ice-cold 1×PBS to the capped tube. Suspend the cells by gentle vortexing. Determine the total number of cells using a haemocytometer slide. Cell numbers should not exceed 2×10⁸. Pellet the cells by centrifugation at 500×g for 10 min in a refrigerated centrifuge (4° C.). Decant the supernatant.

(b) Non-Adherent Cells

For non-adherent cells proceed as follows: decant cell suspension into a 50 ml Falcon tube and centrifuge at 500×g for 10 min in a refrigerated centrifuge (4° C.). Decant the supernatant and add 5 ml of ice-cold 1×PBS to the cells and suspend the cells by gentle vortexing. Pellet the cells by centrifugation at 500×g for 10 min in a refrigerated centrifuge (4° C.). Decant the supernatant and add 5 ml of ice-cold 1×PBS to the Falcon tube. Suspend the cells by gentle vortexing. Determine the total number of cells using a haemocytometer slide. Cell numbers should not exceed 2×10⁸. Pellet the cells by centrifugation at 500×g for 10 min in a refrigerated centrifuge (4° C.). Decant the supernatant.

(c) DNA Extraction and Analysis

Genomic DNA, for both adherent and non-adherent cell lines, was extracted using the Qiagen Genomic DNA extraction kit (Cat No. 10243) as per the manufacturer's instructions. The concentration of genomic DNA recovered was determined using a Beckman model DU64 photospectrometer at a wavelength of 260 nm.

Genomic DNA (10 μg) was digested with appropriate restriction endonucleases and buffer in a volume of 200 μl at 37° C. for approximately 16 hr. Following digestion, 20 μl of 3 M sodium acetate pH 5.2 and 500 μl of absolute ethanol were added to the digest and the solutions mixed by vortexing. The mixture was incubated at −20° C. for 2 hr to precipitate the digested genomic DNA. The DNA was pelleted by centrifugation at 10,000×g for 30 mm at 4° C. The supernatant was removed and the DNA pellet washed with 500 μl of 70% v/v ethanol. The 70% v/v ethanol was removed, the pellet air-dried, and the DNA suspended in 20 μl of water.

Gel loading dye (0.25% w/v bromophenol blue (Sigma); 0.25% w/v xylene cyanol FF (Sigma); 15% w/v Ficoll Type 400 (Pharmacia)) (5 μl) was added to the resuspended DNA and the mixture transferred to a well of 0.7% w/v agarose/TAE gel containing 0.5 μg/ml of ethidium bromide. The digested genomic DNA was electrophoresed through the gel at 14 volts for approximately 16 hr. An appropriate DNA size marker was included in a parallel lane.

The digested genomic DNA was then denatured (1.5 M NaCl, 0.5 M NaOH) in the gel and the gel neutralized (1.5 M NaCl, 0.5 M Tris-HCl pH 7.0). The electrophoresed DNA fragments were then capillary blotted to Hybond NX (Amersham) membrane and fixed by UV cross-linking (Bio Rad GS Gene Linker).

The membrane containing the cross-linked digested genomic DNA was rinsed in sterile water. The membrane was then stained in 0.4% v/v methylene blue in 300 mM sodium acetate (pH 5.2) for 5 min to visualize the transferred genomic DNA. The membrane was then rinsed twice in sterile water and destained in 40% v/v ethanol. The membrane was then rinsed in sterile water to remove ethanol.

The membrane was placed in a Hybaid bottle and 5 ml of pre-hybridization solution added (6×SSPE, 5×Denhardt's reagent, 0.5% w/v SDS, 100 μg/ml denatured, fragmented herring sperm DNA). The membrane was pre-hybridized at 60° C. for approximately 14 hr in a hybridization oven with constant rotation (6 rpm).

Probe (25 ng) was labelled with [α³²P]-dCTP (specific activity 3000 Ci/mmol) using the Megaprime DNA labelling system as per the manufacturer's instructions (Amersham Cat. No. RPN1606). Labelled probe was passed through a G50 Sephadex Quick Spin (trademark) column (Roche, Cat No. 1273973) to remove unincorporated nucleotides as per the manufacturer's instructions.

The heat-denatured labelled probe was added to 2 ml of hybridization buffer (6×SSPE, 0.5% w/v SDS, 100 μg/ml denatured, fragmented herring sperm DNA) pre-warmed to 60° C. The pre-hybridization buffer was decanted and replaced with 2 ml of pre-warmed hybridization buffer containing the labelled probe. The membrane was hybridized at 60° C. for approximately 16 hr in a hybridization oven with constant rotation (6 rpm).

The hybridization buffer containing the probe was decanted and the membrane subjected to several washes:

2×SSC, 0.5% w/v SDS for 5 min at room temperature;

2×SSC, 0.1% w/v SDS for 15 min at room temperature;

0.1×SSC, 0.5% w/v SDS for 30 min at 37° C. with gentle agitation;

0.1×SSC, 0.5% w/v SDS for 1 hour at 68° C. with gentle agitation; and

0.1×SSC for 5 min at room temperature with gentle agitation.

Washing duration at 68° C. varied based on the amount of radioactivity detected with a hand-held Geiger counter.

The damp membrane was wrapped in plastic wrap and exposed to X-ray film (Curix Blue HC-S Plus, AGFA) for 24 to 48 hr and the film developed to visualize bands of probe hybridized to genomic DNA.

8. Immunofluorescent Labelling of Cultured Cells

Glass microscope cover slips (12 mm×12 mm) were flamed with ethanol then submerged in 2 ml growth medium, two per well, in six-well plates. Cells were added to wells in 1-2 ml medium to give a density of cells after 16 hr growth such that cells remain isolated (200,000 to 500,000 per well depending on size and growth rate of cells). Without removing the cover slips from wells, the medium was aspirated and cells were washed with PBS. For fixation, cells were treated for 1 hr with 4% w/v paraformaldehyde (Sigma) in PBS then washed three times with PBS. Fixed cells were permeabilized with 0.1% v/v Triton X-100 (Sigma) in PBS for 5 min then washed three times with PBS. Cells on cover slips were blocked on one drop (about 100 μl) of 0.5% w/v bovine serum albumin Fraction V (BSA, Sigma) for 10 min. Cover slips were then placed for at least 1 h on 25 μl drops of primary mouse monoclonal antibody which had been diluted 1/100 in 0.5% v/v BSA in PBS. Cells on cover slips were then washed three times with 100 μl of 0.5% v/v BSA in PBS for about 3 min each before being placed for 30 min to 1 hr on 25 μl drops of Alexa Fluor (registered trademark) 488 goat anti-mouse IgG conjugate (Molecular Probes) secondary antibody diluted 1/100 in 0.5% v/v BSA in PBS. Cells on cover slips were then washed three times with PBS. Cover slips were mounted on glass microscope slides, three to the slide, in glycerol/DABCO [25 mg/ml DABCO (1,4diazabicyclo(2.2.2)octane (Sigma D 2522)) in 80% v/v glycerol in PBS] and examined with a 100×oil immersion objective under UV illumination at 500-550 nm.

9. Composition of Media Used in Experimental Protocols

The compositions of DMEM, OPTI-MEM I (registered trademark) Reduced Serum Medium, PBS and Trypsin-EDTA used are set out in Example 1.

(a) RPMI 1640 Medium

A commercial formulation of RPMI 1640 medium (Cat. No. 21870) was used and obtained from Life Technologies. The liquid formulation was:

| | | |
|---|---|---|
| Ca(NO₃)₂.4H₂O | 100 | mg/l |
| KCI | 400 | mg/l |
| MgSO₄ (anhyd) | 48.84 | mg/l |
| NaCl | 6,000 | mg/l |
| NaHCO₃ | 2,000 | mg/l |
| NaH₂PO₄ (anhyd) | 800 | mg/l |
| D-glucose | 2,000 | mg/l |
| Glutathione (reduced) | 1.0 | mg/l |
| Phenol Red | 5 | mg/l |
| L-Arginine | 200 | mg/l |
| L-Asparagine (free base) | 50 | mg/l |
| L-Aspartic Acid | 20 | mg/l |
| L-Cystine.2HCI | 65 | mg/l |
| L-Glutamic Acid | 20 | mg/l |
| Glycine | 10 | mg/l |
| L-Histidine (free base) | 15 | mg/l |
| L-Hydroxyproline | 20 | mg/l |
| L-Isoleucine | 50 | mg/l |
| L-Leucine | 50 | mg/l |
| L-Lysine.HCI | 40 | mg/l |
| L-Methionine | 15 | mg/l |
| L-Phenylalanine | 15 | mg/l |
| L-Proline | 20 | mg/l |
| L-Serine | 30 | mg/l |
| L-Threonine | 20 | mg/l |
| L-Tryptophan | 5 | mg/l |
| L-Tyrosine.2Na2H₂O | 29 | mg/l |
| L-Valine | 20 | mg/l |
| Biotin | 0.2 | mg/l |
| D-Ca Pantothenate | 0.25 | mg/l |
| Choline chloride | 3 | mg/l |
| Folic Acid | 1 | mg/l |
| i-Inositol | 35 | mg/l |
| Niacinamide | 1 | mg/l |
| Para-aminobenzioe Acid | 1 | mg/l |
| Pyridoxine HCI | 1 | mg/l |
| Riboflavin | 0.2 | mg/l |
| Thiamine HCI | 1 | mg/l |
| Vitamin B₁₂ | 0.005 | mg/l |

### EXAMPLE 11

Preparation of Plasmid Construct Cassettes for Use in Achieving Co-Suppression

1. Generic RNA Isolation, cDNA Synthesis and PCR Protocol

Total RNA was purified from the indicated cell lines using an RNeasy Mini Kit according to the manufacturer's protocol (Qiagen). To prepare cDNA, this RNA was reverse transcribed using Omniscript Reverse Transcriptase (Qiagen). Two micrograms of total RNA was reverse transcribed using 1 μM oligo dT (Sigma) as a primer in a 20 μl reaction according to the manufacturer's protocol (Qiagen).

To amplify specific products, 2 μl of this mixture was used as a substrate for PCR amplification, which was performed using HotStarTaq DNA polymerase according to the manufacturer's protocol (Qiagen). PCR amplification conditions involved an initial activation step at 95° C. for 15 mins, followed by 35 amplification cycles of 94° C. for 30 secs, 60° C. for 30 secs and 72° C. for 60 secs, with a final elongation step at 72° C. for 4 mins.

PCR products to be cloned were usually purified using a QIAquick PCR Purification Kit (Qiagen); in instances where multiple fragments were generated by PCR, the fragment of the correct size was purified from agarose gels using a QIAquick Gel Purification Kit (Qiagen) according to the manufacturer's protocol.

Amplification products were then cloned into pCR (registered trademark)2.1-TOPO (Invitrogen) according to the manufacturer's protocol.

2. Generic Cloning Techniques

To prepare the constructs described below, insert fragments were excised from intermediate vectors using restriction enzymes according to the manufacturer's protocols (Roche) and fragments purified from agarose gels using QIAquick Gel Purification Kits (Qiagen) according to the manufacturer's protocol. Vectors were usually prepared by restriction digestion and treated with Shrimp Alkaline Phosphatase according to the manufacturer's protocol (Amersham). Vector and inserts were ligated using T4 DNA ligase according to the manufacturer's protocols (Roche) and transformed into competent E. coli strain DH5a using standard procedures (Sambrook et al.; 1984).

3. Constructs

(a) Commercial Plasmids

Plasmid pEGFP-N1

Plasmid pEGFP-N1 (FIG. 1; Clontech) contains the CMV IE promoter operably connected to an open reading frame encoding a red-shifted variant of the wild-type GFP which has been optimized for brighter fluorescence. The specific GFP variant encoded by pEGFP-N1 has been disclosed by Cormack et al. (1996). Plasmid pEGFP-N1 contains a multiple cloning site comprising BglII and BamHI sites and many other restriction endonuclease cleavage sites, located between the CMV IE promoter and the EGFP open reading frame. The plasmid pEGFP-N1 will express the EGFP protein in mammalian cells. In addition, structural genes cloned into the multiple cloning site will be expressed as EGFP fusion polypeptides if they are in-frame with the EGFP-encoding sequence and lack a functional translation stop codon. The plasmid further comprises an SV40 polyadenylation signal downstream of the EGFP open reading frame to direct proper processing of the 3′-end of mRNA transcribed from the CMV IE promoter sequence (SV40 pA). The plasmid further comprises the SV40 origin of replication functional in animal cells; the neomycin-resistance gene comprising SV40 early promoter (SV40-E in FIG. 1) operably connected to the neomycin/kanamycin-resistance gene derived from Tn5 Kan/Neo in FIG. 1) and the HSV thymidine kinase polyadenylation signal, for selection of transformed cells on kanamycin, neomycin or geneticin; the pUC19 origin of replication which is functional in bacterial cells and the f1 origin of replication for single-stranded DNA production.

Plasmid pBluescript II SK⁺

Plasmid pBluescript II SK⁺ is commercially available from Stratagene and comprises the lacZ promoter sequence and lacZ-α transcription terminator, with multiple restriction endonuclease cloning sites located there between. Plasmid pBluescript II SK⁺ is designed to clone nucleic acid fragments by virtue of the multiple restriction endonuclease cloning sites. The plasmid further comprises the Co1E1 and f1 origins of replication and the ampicillin-resistance gene.

Plasmid pCR (Registered Trademark) 2.1

Plasmid pCR2.1 is a commercially-available, T-tailed vector from Invitrogen and comprises the lacZ promoter sequence and lacZ-α transcription terminator, with a cloning site for the insertion of structural gene sequences there between. Plasmid pCR (registered trademark) 2.1 is designed to clone nucleic acid fragments by virtue of the A-overhang frequently synthesized by Taq polymerase during the polymerase chain reaction. The plasmid further comprises the Co1E1 and f1 origins of replication and kanamycin-resistance and ampicillin-resistance genes.

Plasmid pCR (Registered Trademark) 2.1-TOPO

Plasmid pCR (registered trademark) 2.1-TOPO is a commercially available T-tailed vector from Invitrogen and comprises the lacZ promoter sequence and lacZ-α transcription terminator, with multiple restriction endonuclease cloning sites located there between. Plasmid pCR (registered trademark) 2.1-TOPO is provided with covalently bound topoisomerase I enzyme for fast cloning. The plasmid further comprises the Co1E1 and f1 origins of replication and the kanamycin and ampicillin resistance genes.

Plasmid pPUR

Plasmid pPUR is commercially available from Clontech and comprises the SV40 early promoter operably connected to an open reading frame encoding the Streptomyces alboniger puromycin-N-acetyl-transferase (pac) gene (de la Luna and Ortin, 1992). The plasmid further comprises an SV40 polyadenylation signal downstream of the pac open reading frame to direct proper processing of the 3′-end of mRNA transcribed from the SV40 E promoter sequence. The plasmid further comprises a bacterial replication origin and the ampicillin resistance (β-lactamase) gene for propagation in E. coli.

(b) Intermediate Cassettes

Plasmid TOPO.BGI2

Plasmid TOPO.BGI2 comprises the human β-globin intron number 2 (BGI2) placed in the multiple cloning region of plasmid pCR (registered trademark) 2.1-TOPO. To produce this plasmid, the human β-globin intron number 2 was amplified from human genomic DNA using the amplification primers:

GD1 GAG CTC TTC AGG GTG AGT CTA TGG GAC CC [SEQ ID NO: 1]

and

GA1 CTG CAG GAG CTG TGG GAG GAA GAT AAG AG [SEQ ID NO: 2]

and cloned into plasmid pCR (registered trademark) 2.1-TOPO to make plasmid TOPO.BGI2. BGI2 is a functional intron sequence that is capable of being post-transcriptionally cleaved from RNA transcripts containing it in mammalian cells.

Plasmid TOPO.PUR

Plasmid TOPO.PUR comprises the SV40 E promoter, the puromycin-N-acetyl-transferase gene, and the SV40 polyadenylation signal sequence from the plasmid pPUR placed in the multiple cloning region of plasmid pCR (registered tradmark) 2.1-TOPO. To produce this plasmid, the region of plasmid pPUR containing the SV40 E promoter, the puromycin-N-acetyl-transferase gene, and the SV40 polyadenylation signal sequence was amplified from plasmid pPUR (Clontech) using the amplification primers:

AflIII-pPUR-Fwd TCT CCT TAC GCG TCT GTG CGG TAT [SEQ ID NO: 3] and

AflIII-pPUR-Rev ATG AGG ACA CGT AGG AGC TTC CTG [SEQ ID NO: 4]

and cloned into plasmid pCR (registered trademark) 2.1-TOPO to make plasmid TOPO.PUR.

(c) Plasmid Cassettes

Plasmid pCMV.cass

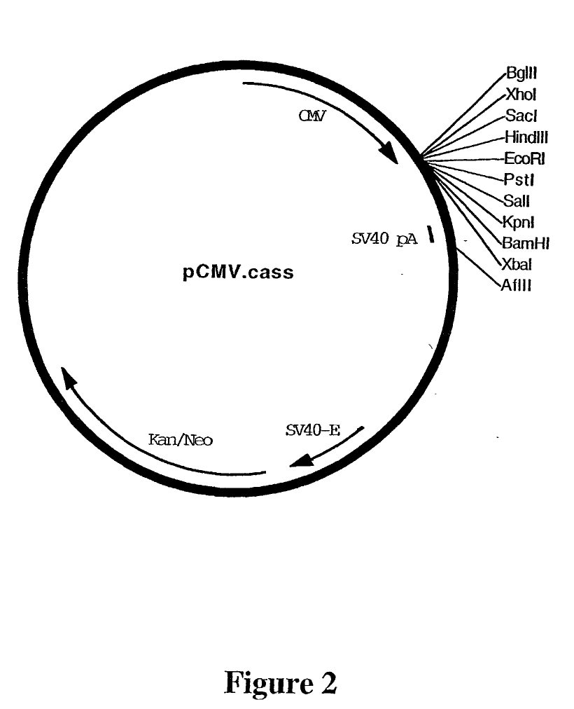

Plasmid pCMV.cass FIG. 2) is an expression cassette for driving expression of a structural gene sequence under control of the CMV-IE promoter sequence. Plasmid pCMV.cass was derived from pEGFP-N1 (FIG. 1) by deletion of the EGFP open reading frame as follows: Plasmid pEGFP-N1 was digested with PinAI and NotI, blunt-ended using PfuI DNA polymerase and then religated. Structural gene sequences are cloned into pCMV.cass using the multiple cloning site, which is identical to the multiple cloning site of pEGFP-N1, except it lacks the PinAI site.

Plasmid pCMV.BGI2.cass

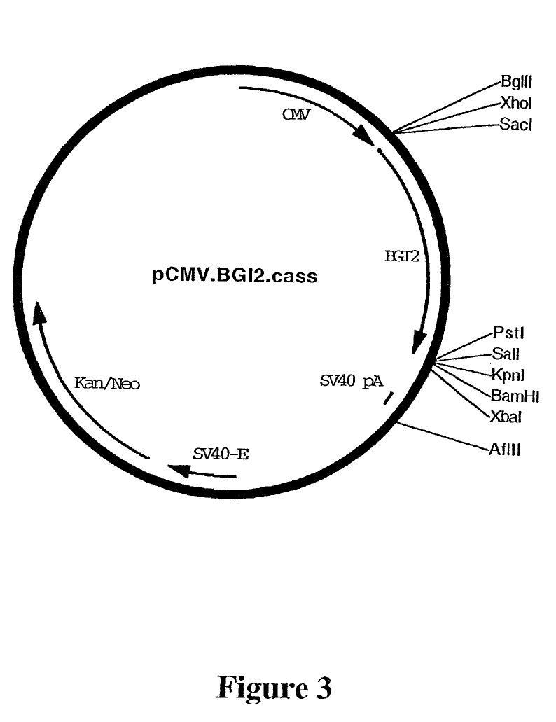

To create pCMV.BG12.cass (FIG. 3), the human β-globin intron sequence was isolated as a SacI/PstI fragment from TOPO.BGI2 and cloned between the SacI and PstI sites of pCMV.cass. In pCMV.BGI2.cass, any RNAs transcribed from the CMV promoter will include the human β-globin intron 2 sequences; these intron sequences will presumably be excised from transcripts as part of the normal intron processing machinery, since the intron sequences include both the splice donor and splice acceptor sequences necessary for normal intron processing.

### EXAMPLE 12

Co-Suppression of Green Fluorescent Protein in Porcine Kidney Type 1 Cells In Vitro

1. Culturing of Cell Lines

PK-1 cells (derived from porcine kidney epithelial cells) were grown as adherent monolayers using DMEM supplemented with 10% v/v FBS, as described in Example 10, above.

2. Preparation of Genetic Constructs

(a) Interim Plasmids

Plasmid pBluescript.EGFP

Plasmid pBluescript.EGFP comprises the EGFP open reading frame derived from plasmid pEGFP-N1 (FIG. 1, refer to Example 11) placed in the multiple cloning region of plasmid pBluescript II SK⁺. To produce this plasmid, the EGFP open reading frame was excised from plasmid pEGFP-N1 by restriction endonuclease digestion using the enzymes NotI and XhoI and ligated into NotI/XhoI digested pBluescript II SK⁺.

Plasmid pCR.Bgl-GFP-Bam

Plasmid pCR.Bgl-GFP-Bam comprises an internal region of the EGFP open reading frame derived from plasmid pEGFP-N1 (FIG. 1) placed in the multiple cloning region of plasmid pCR2.1 (Invitrogen, see Example 11). To produce this plasmid, a region of the EGFP open reading frame was amplified from pEGFP-N1 using the amplification primers:

Bgl-GFP: CCC GGG GCT TAG TGT AAA ACA GGC TGA GAG [SEQ ID NO: 5]

and

GFP-Bam: CCC GGG CAA ATC CCA GTC ATT TCT TAG AAA [SEQ ID NO: 6]

and cloned into plasmid pCR2.1, according to the manufacturer's directions (nvitrogen). The internal EGFP-encoding region in plasmid pCR.Bgl-GFP-Bam lacks functional translational start and stop codons.

Plasmid pCMV GFP.BGI2.PFG

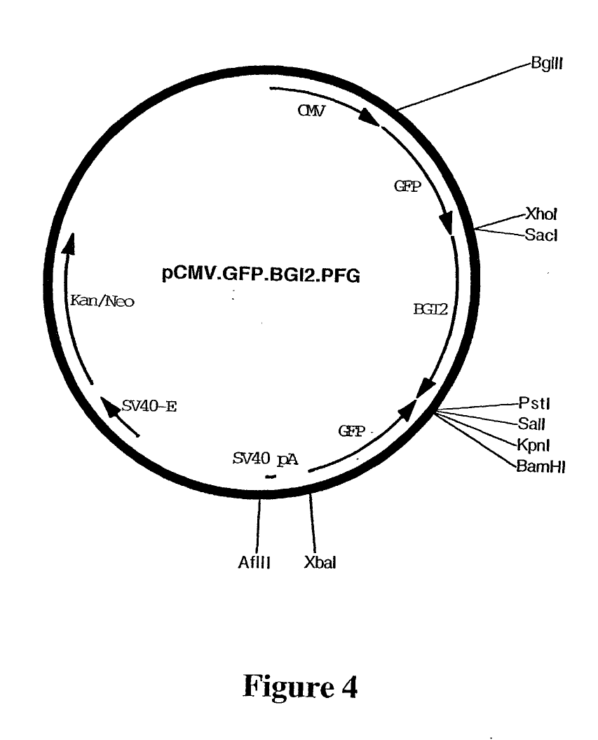

Plasmid pCMV.GFP.BGI2.PFG (FIG. 4) contains an inverted repeat or palindrome of an internal region of the EGFP open reading frame that is interrupted by the insertion of the human β-globin intron 2 sequence therein. Plasmid pCMV.GFP.BGI2.PFG was constructed in successive steps: (i) the GFP sequence from plasmid pCR.Bgl-GFP-Bam was sub-cloned in the sense orientation as a BglII-to-BamHI fragment into BglII-digested pCMV.BGI2.cass (FIG. 3, refer to Example 11) to make plasmid pCMV.GFP.BGI2, and (ii) the GFP sequence from plasmid pCR.Bgl-GFP-Bam was sub-cloned in the antisense orientation as a BglII-to-BamHI fragment into BamHI-digested pCMV.GFP.BGI2 to make the plasmid pCMV.GFP.BGI2.PFG.

(b) Test Plasmids

Plasmid pCM.EGFP

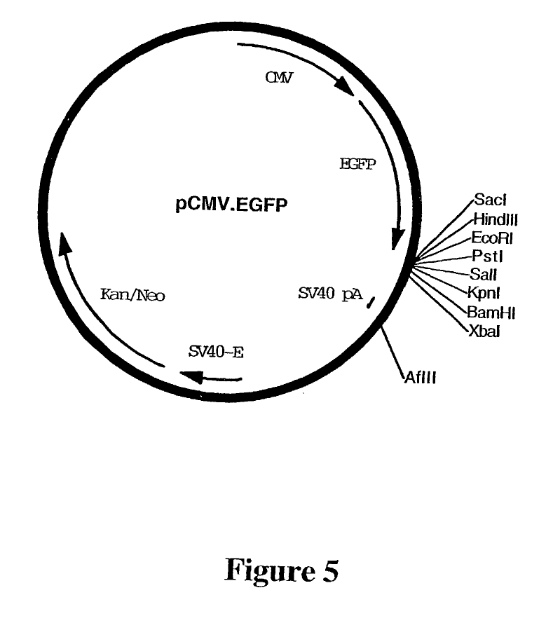

Plasmid pCMV.EGFP (FIG. 5) is capable of expressing the entire EGFP open reading frame under the control of CMV-IE promoter sequence. To produce pCMV.EGFP, the EGFP sequence from pBluescript.EGFP, above, was sub-cloned in the sense orientation as a BamHI-to-SacI fragment into BglIISacI-digested pCMV.cass (FIG. 2, refer to Example 11) to make plasmid pCMV.EGFP.

Plasmid pCMV^{pur}.BGI2.cass

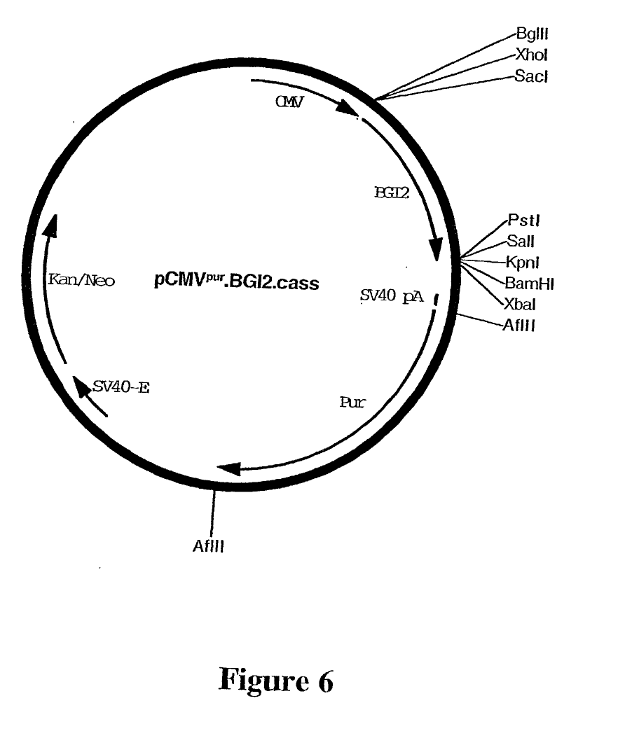

Plasmid pCMV^{pur}.BGI2.cass (FIG. 6) contains a puromycin resistance selectable marker gene in pCMV.BGI2.cass (FIG. 3) and is used as a control in these experiments. To create PCMV^{pur}.BGI2.cass, the puromycin resistance gene from TOPO.PUR (Example 10) was cloned as an AflII fragment into AflII-digested pCMV.BGI2.cass.

Plasmid pCMV^{pur}.GFP.BGI2.PFG

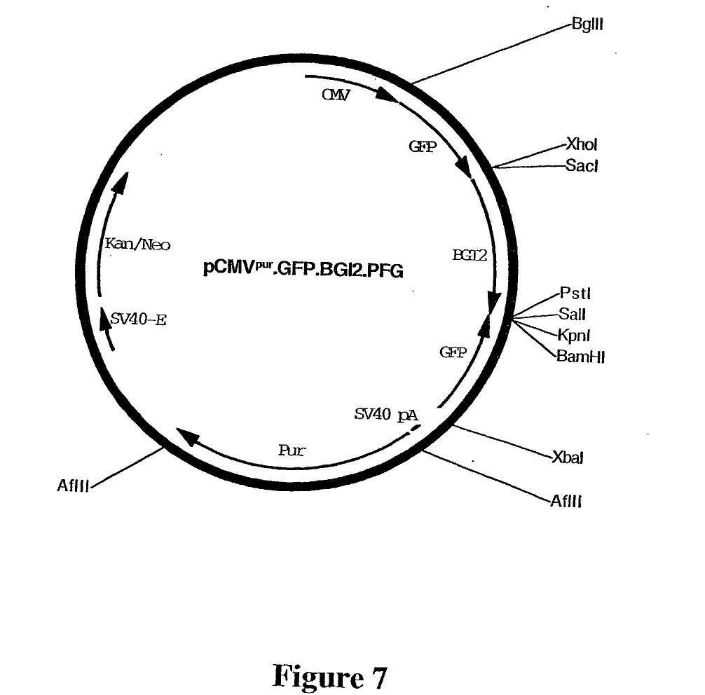

Plasmid pCMV^{pur}.GFP.BGI2.PFG (FIG. 7) contains an inverted repeat or palindrome of an internal region of the EGFP open reading frame that is interrupted by the insertion of the human β-globin intron 2 sequence therein and a puromycin resistance selectable marker gene. Plasmid pCMV^{pur}.GFP.BGI2.PFG was constructed by cloning the puromycin resistance gene from TOPO.PUR (Example 10) as an AflII fragment into AflII-digested pCMV.GFP.BGI2.PFG (FIG. 4).

3. Detection of Co-Suppression Phenotype

(a) Insertion of EGFP-Expressing Transgene into PK-4 Cells

Transformations were performed in 6 well tissue culture vessels. Individual wells were seeded with 4×10⁴ PK-1 cells in 2 ml of DMEM, 10% v/v FBS and incubated at 37° C., 5% v/v CO₂ until the monolayer was 60-90% confluent, typically 16 to 24 hr.

To transform a single plate (6 wells), 12 μg of pCMV.EGFP (FIG. 5) plasmid DNA and 108 μl of GenePORTER2 (trademark) (Gene Therapy Systems) were diluted into OPTI-MEM-I (registered trademark) to obtain a final volume of 6 ml and incubated at room temperature for 45 min.

The tissue growth medium was removed from each well and the monolayers therein washed with 1 ml of 1×PBS. The monolayers were overlayed with 1 ml of the plasmid DNA/GenePORTER2 (trademark) conjugate for each well and incubated at 37° C., 5% v/v CO₂ for 4.5 hr.

OPTI-MEM-I (registered trademark) (1 ml) supplemented with 20% v/v FBS was added to each well and the vessel incubated for a further 24 hr, at which time the monolayers were washed with 1×PBS and medium was replaced with 2 ml of fresh DMEM including 10% v/v FBS. Cells transformed with pCMV.EGFP were examined after 24-48 hr for transient EGFP expression using fluorescence microscopy at a wavelength of 500-550 nm.

Forty-eight hr after transfection the medium was removed, the cell monolayer washed with 1×PBS and 4 ml of fresh DMEM containing 10% v/v FBS, supplemented with 1.5 mg/ml genetecin (Life Technologies), was added to each well. Genetecin was included in the medium to select for stably transformed cell lines. The DMEM, 10% v/v FBS, 1.5 mg/ml genetecin medium was changed every 48-72 hr. After 21 days of selection, stable, EGFP-expressing PK-1 colonies were apparent.

Individual colonies of stably transfected PK-1 cells were cloned, maintained and stored as described in Generic Techniques in Example 10, above.

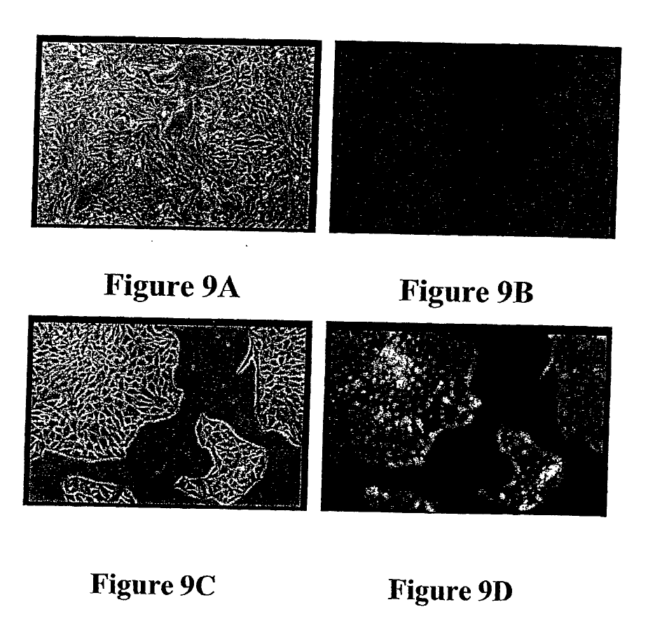

A number of parental cell lines were transformed with pCMV.EGFP. In many of these, GFP expression was either extremely low or completely undetectable as listed in Table 2 and shown in FIGS. 9A, 9B, 9C and 9D.

TABLE 2

| | | Number of cell lines |
|---|---|---|
| Parental | Number of cloned | with extremely low |
| Cell line | lines examined | or undetectable GFP |
| PK-1 | 59 | 2 |
| MM96L | 12 | 4 |
| B16 | 12 | 10 |
| MDAMB468 | 11 | 1 |

These data indicated that inactivation of GFP occurred frequently in different types of cell lines, established from three different species.

(b) Post-Transcriptional Silencing of EGFP-Expressing Transgene in PK-1 Cells

To study the onset of post-transcriptional gene silencing (PTGS) of the EGFP-expressing transgene, cells from 12 stable EGFP-expressing PK-1 lines (PK-1/EGFP) were transfected with the construct pCMV^{pur}.GFP.BGI2.PFG (FIG. 7). Two controls were also included. The first control was a replicate of each stable line transformed with the plasmid pCMV^{pur}.BGI2.cass (FIG. 6) The second control was a replicate untransfected PK-1/EGFP line.

The transformation of PK-1 cells with pCMV^{pur}.GFP.BGI2.PFG and pCMV^{pur}.IBGI2.cass was performed in 6-well tissue culture vessels, in triplicate, using the same method as described above in (a).

Forty-eight hr after transfection the medium was removed, the cell monolayer washed with PBS (as above) and 4 ml of fresh DMEM containing 10% v/v FBS and 1 mg/ml geneticin (GGM) were added to each well of cells. In addition, where the cells were transfected with either pCMV^{pur}.BGI2.cass or pCMV^{pur}.GFP.BGI2.PFG, the GGM was further supplemented with 1.0 μg/ml puromycin; puromycin was included in the medium to select for stably transformed cell lines. After 21 days of selection, co-transformed silenced colonies were apparent. Following transfection, all replicates were inspected microscopically for the presence of PTGS, as indicated by the absence of the EGFP-expressing phenotype in cells transformed with PCMV^{pur}.GFP.BGI2.PFG but not in cells transformed with pCMV^{pur}.BGI2.cass or transfected replicate controls.

3. Analysis by Nuclear Transcription Run-On Assays

To detect transcription of the transgene RNA in the nucleus of PK-1 cells, nuclear transcription run-on assays are performed on cell-free nuclei isolated from actively dividing cells. The nuclei are obtained according to the cell nuclei isolation protocol set forth in Example 10, above.

Analyses of nuclear RNA transcripts for the transgene EGFP from the transfected plasmid pCMV.EGFP and the transgene GFP.BGI2.PFG from the co-transfected plasmid pCMV^{pur}.GFP.BGI2PFG are performed according to the nuclear transcription run-on protocol set forth in Example 10, above.

Rates of transcription in the nuclei of all PK-1 cells analyzed—whether transfected with plasmid pCMV.EGFP or with the transgene GFP.BGI2.PFG—are not substantially different from rates found in nuclei of either the untransfected PK-1/EGFP control line or the control line transformed with the plasmid pCMV^{pur}.BGI2.cass.

5. Comparison of mRNA in Non-Transformed and Co-Suppressed Lines

Messenger RNA for EGFP from the plasmid pCMV.EGFP and RNA transcribed from the transgene GFP.BGI2.PFG are analyzed according to the protocol set forth in Example 10, above.

6. Southern Analysis

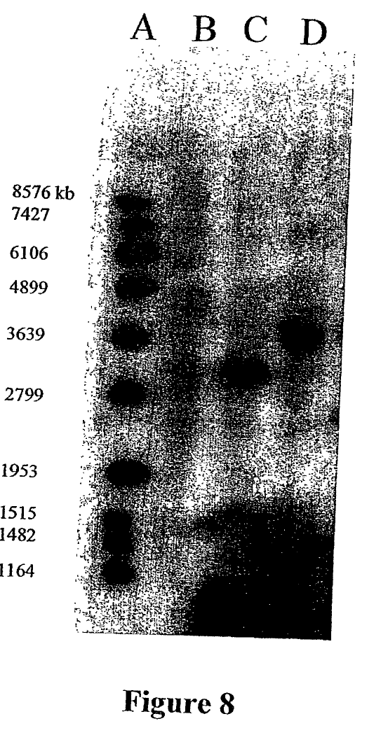

Individual transgenic PK-1 cell lines (transfected and co-transfected) are analyzed by Southern blot analysis to confirm integration and determine copy number of the transgenes. The procedure is carried out according to the protocol set forth in Example 10, above. An example is illustrated in FIG. 8.

### EXAMPLE 13

Co-Suppression of Bovine Enterovirus in Madin Darby Bovine Kidney Type CRIB-1 Cells In Vitro

1. Culturing of Cells Lines

CRIB-1 cells (derived from bovine kidney epithelial cells) were grown as adherent monolayers using DMEM supplemented with 10% v/v Donor Calf Serum (DCS; Life Technologies), as described in Example 10, above. Cells were always grown in incubators at 37° C. in an atmosphere containing 5% v/v CO₂.

2. Preparation of Genetic Constructs

(a) Interim Plasmid

Plasmid pCR.BEV2

The complete Bovine enterovirus (BRV) RNA polymerase coding region was amplified from a full-length cDNA clone encoding same, using primers:

BEV-1 CGG CAG ATC CTA ACA ATG GCA GGA CAA ATC GAG TAC ATC [SEQ ID NO: 7]

and

BEV-3 GGG CGG ATC CTT AGA AAG AAT CGT ACC AC [SEQ ID) NO: 8].

Primer BEV-1 comprises a BglII restriction endonuclease site at positions 4 to 9 inclusive, and an ATG start site at positions 16-18 inclusive. Primer BEV-3 comprises a BamHI restriction enzyme site at positions 5 to 10 inclusive and the complement of a TAA translation stop signal at positions 11 to 13 inclusive. As a consequence, an open reading frame comprising a translation start signal and a translation stop signal is contained between the BglII and BamHI restriction sites. The amplified fragment was cloned into pCR2.1 to produce plasmid pCR.BEV2.

Plasmid pBS.PFGE

Plasmid pBS.PFGE contains the EGFP coding sequences from pEGFP-N1 cloned into the polylinker of pBluescript II SK⁺. To generate this plasmid, the EGFP coding sequences from pEGFP-N1 was cloned as a NotI-to-SacI fragment into NotI/SacI-digested pBluescript II SK⁺.

(b) Test Plasmids

Plasmid PCMV.EGFP

Plasmid pCMV.EGFP (FIG. 5) is capable of expressing the entire EGFP open reading frame and is used in this and subsequent examples as a positive transfection control (refer to Example 12, 2(b)).

Plasmid pCMV.BEV2.BGI2.2VEB

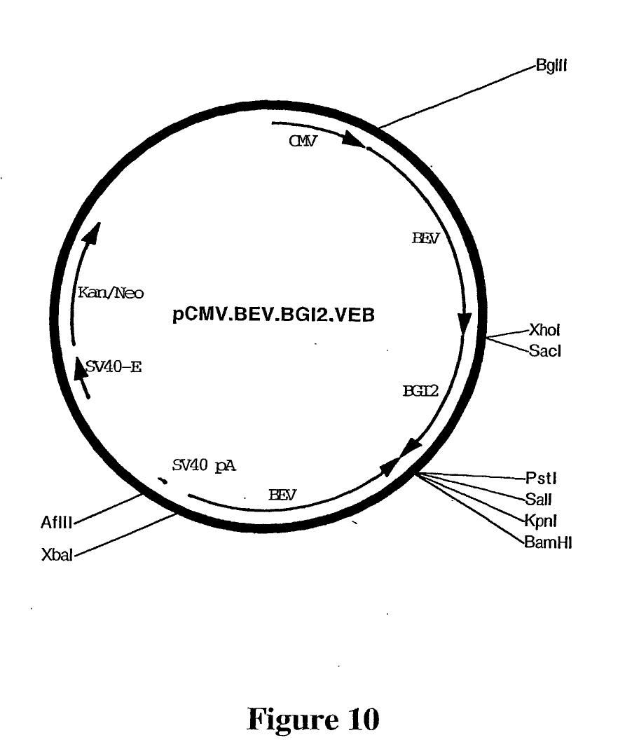

Plasmid pCMV.BEV2.BGI2.2VEB (FIG. 10) contains an inverted repeat or palindrome of the BEV polymerase coding region that is interrupted by the insertion of the human β-globin intron 2 sequence therein. Plasmid pCMV.BEV2.BGI2.2VEB was constructed in successive steps: (i) the BEV2 sequence from plasmid pCR.BEV2 was sub-cloned in the sense orientation as a BglII-to-BamHI fragment into BglII-digested pCMV.BGI2.cass (Example 11) to make plasmid pCMV.BEV2.BGI2, and (ii) the BEV2 sequence from plasmid pCR-BEV2 was sub-cloned in the antisense orientation as a BglII-to-BamHI fragment into BamHI-digested pCMV.BEV2.BGI2 to make plasmid pCMV.BEV2.BGI2.2VEB.

Plasmid pCMV.BEV.EGFP.VEB

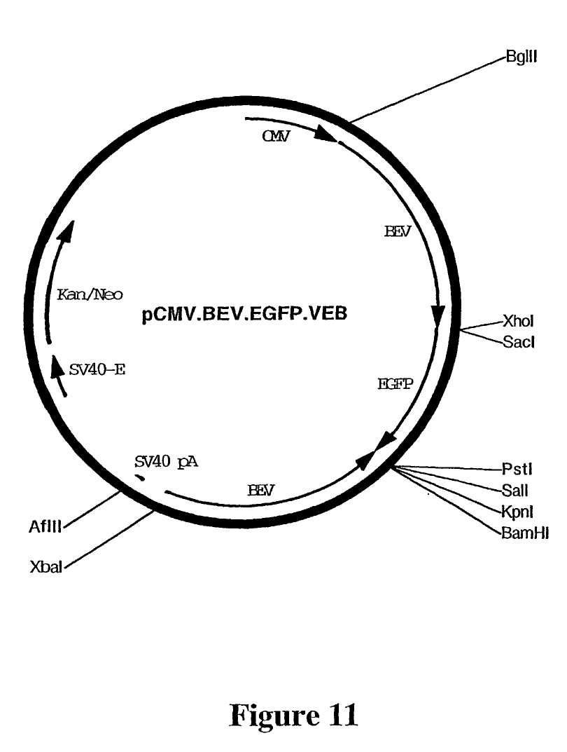

Plasmid pCMV.BEV.EGFP.VEB (FIG. 11) contains an inverted repeat or palindrome of the BEV polymerase coding region that is interupted by EGEP coding sequences which act as a stuffer fragment. To generate this plasmid, the EGFP coding sequence from pBS.PFGE was isolated as an EcoRI fragment and cloned into EcoRI-digested pCMV.cass in the sense orientation relative to the CMV promoter to generate pCMV.EGFP.cass. Plasmid pCMV. BEV.EGFP.VEB was constructed in successive steps: (i) the BEV polymerase sequence from plasmid pCR.BEV2 was subcloned in the sense orientation as a BglII-to-BamHI fragment into BglII-digested pCMV.EGFP.cass to make plasmid pCMV.BEV.EGFP, and (ii) the BEV polymerase sequence from plasmid pCR.BEV2 was sub-cloned in the antisense orientation as a BglII-to-BamHI fragment into BamHI-digested pCMV.BEV.EGFP to make plasmid pCMV. BEV.EGFP.VEB.

3. Detection of Co-Suppression Phenotype

(a) Insertion of Bovine Enterovirus RNA Polymerase-Expressing Transgene into CRIB-1 Cells

Transformations were performed in Swell tissue culture vessels. Individual wells were seeded with 2×10⁵ CRIB-1 cells in 2 ml of DMEM, 10% v/v DCS and incubated at 37° C., 5% v/v CO₂ until the monolayer was 60-90% confluent, typically 16 to 24 hr.

The following solutions were prepared in 10 ml sterile tubes:

Solution A: For each transfection, 1 μg of DNA (pCMV.BEV2.BGL2.2VEB or pCMV.EGFP—Transfection Control) was diluted into 100 μl of OPTI-MEM-I (registered trademark) Reduced Serum Medium (serum-free medium) and;

Solution B: For each transfection, 10 μl of LIPOFECTAMINE (trademark) Reagent was diluted into 100 μl OPTI-MEM-I (registered trademark) Reduced Serum Medium.

The two solutions were combined and mixed gently, and incubated at room temperature for 45 min to allow DNA-liposome complexes to form. While complexes formed, the CRIB-1 cells were rinsed once with 2 ml of OPTI-MEM I (registered tradmark) Reduced Serum Medium.

For each transfection, 0.8 ml of OPTI-MEM I (registered trademark) Reduced Serum Medium was added to the tube containing the complexes, the tube mixed gently, and the diluted complex solution overlaid onto the rinsed CRIB-1 cells. Cells were then incubated with the complexes at 37° C. and 5% v/v CO₂ for 16 to 24 hr.

Transfection mixture was then removed and the CRIB-1 monolayers overlaid with 2 ml of DMEM, 10% v/v DCS. Cells were incubated at 37° C. and 5% v/v CO₂ for approximately 48 hr. To select for stable transformants, the medium was replaced every 72 hr with 4 ml of DMEM, 10% v/v DCS, 0.6 mg/ml geneticin. Cells transformed with the transfection control pCMV.EGFP were examined after 24-48 hr for transient EGFP expression using fluorescence microscopy at a wavelength of 500-550 nm. After 21 days of selection, stably transformed CRIB-1 colonies were apparent.

Individual colonies of stably transfected CRIB-1 cells were cloned, maintained and stored as described in Generic Techniques in Example 10, above.

(b) Determination of Bovine Enterovirus Titre

The BEV isolate used in these experiments was a cloned isolate, K2577. The titre of this original viral stock was unknown. To amplify BEV virus from this stock, cells were infected with 5 μl of viral stock per well and the virus allowed to replicate for, 48 hr, as described below. Culture medium was harvested at this time and transferred to a screw capped tube. Dead cells and debris were then removed by centrifugation at 3,500 rpm for 15 min at 4° C. in a Sigma 3K18 centrifuge. The supernatant was decanted into a fresh tube and centrifuged at 20,000 rpm for 30 min at 4° C. in a Beckman J2-M1 centrifuge to remove remaining debris. The supernatant was decanted and this new BEV stock titred as described below and stored at 4° C.

Absolute:

In a 6-well tissue culture plate, seed 2.5×10⁵ CRIB-1 cells per well in 2 ml DMEM, 10% v/v DCS. Incubate the cells at 37° C. in an atmosphere containing 5% v/v CO₂ until the cells are 90-100% confluent.

Dilute BEV in serum-free medium DMFM at dilutions of 10⁻¹ to 10⁻⁹. Aspirate the medium from the CRIB-1 monolayers. Overlay the monolayer with 2 ml of 1×PBS and gently rock the tissue culture vessel to wash the monolayer. Aspirate the PBS from the monolayer and repeat the wash once more.

Immediately add 1 ml of the diluted virus solutions (10⁻⁴ to 10 ⁻⁹) directly onto the rinsed CRIB-1 cells, using one dilution per well in duplicate. Incubate the CRIB-1 cells with BEV for 1 hour at 37° C. and 5% v/v CO₂ with gentle agitation. Aspirate the viral inoculum and overlay infected cells with 3 ml of nutrient agar (1% Noble Agar in DMEM). The Noble Agar is made up 2% w/v in sterile distilled water and the DMEM as 2×DMEM. Melt the Noble Agar and equilibrate to 50° C. in a water-bath for 1 hour. Equilibrate the 2×DMEM to 37° C. in a water-bath for 15 min prior to use. Mix the two solutions 1:1 and use to overlay infected cells.

Allow the nutrient agar overlay to set and incubate inverted at 37° C. and 5% v/v CO₂ for 18-24 hr. Following incubation, overlay each well with 3 ml of Neutral Red Agar (1.7 ml Neutral Red Solution (Life Technologies)/100 ml Nutrient Agar). Allow the Neutral Red Agar overlay to set and incubate the 6 well plates in an inverted position in the dark at 37° C. and 5% v/v CO₂ for 18-24 hr. Count the number of plaques 24 hr after addition of Neutral Red Agar to determine the titre of the BEV viral stock.

Empirical:

In a 24-well tissue culture plate, 4×10⁴ CRIB-1 cells were seeded per well in 800 μl DMEM, 10% v/v DCS. The cells were incubated at 37° C. in an atmosphere containing 5% v/v CO₂ until they were 90-100% confluent.

From concentrated BEV viral stock, BEV was diluted in serum-free DMEM at dilutions of 10⁻¹ to 10⁻⁹. The medium was aspirated from the CRIB-i monolayers and the monolayer overlaid with 800 μl of 1×PBS and washed by gently rocking the tissue culture vessel. PBS was aspirated from the monolayer and the wash repeated.

200 μl of the diluted virus solutions (10⁻³ to 10⁻⁹) was added immediately directly onto the rinsed CRIB-1 cells using one dilution per well in duplicate. The CRIB-1 cells were incubated with BEV for 24 hr at 37° C. and 5% v/v CO₂ and each well inspected microscopically for cell lysis. A further 600 μl of serum-free DMEM was then added to each well. After a further 24 hr, each well was inspected microscopically for cell lysis. The correct dilution is the minimum viral concentration that kills most of the CRIB-1 cells after 24 hr and all cells after 48 hr.

(c) Bovine Enterovirus Challenge of CRIB-1 Cells Transformed with pCMVBEV2.BGI2.2VEB

In a 24-well tissue culture plate, 4×104 CRIB-1 cells per well were seeded in triplicate, in 800 μl DMEM, 10% v/v DCS The cells were incubated at 37° C. in an atmosphere containing 5% v/v CO₂ until they were 90-100% confluent.

From concentrated BEV viral stock, BEV virus was diluted in serum-free DUEM at the correct dilution as determined by absolute or empirical measurement. In addition, the BEV viral stock was diluted to one log above and below the correct dilution (typically 10⁻⁴ to 10⁻⁶). The medium was aspirated from the CRIB-1 monolayers and the monolayers overlaid with 800 μl of 1×PBS and washed gently by rocking the tissue culture vessel. PBS was aspirated from the monolayer and the wash repeated.

200 μl of the diluted virus solutions (one dilution per replicate) was added immediately directly onto the rinsed CRIB-1 cells. The cells were incubated with BEV for 24 hr at 37° C. and 5% v/v CO₂, and each well inspected microscopically for cell lysis. A further 600 μl of serum-free DMEM was added to each well. After a further 24 hr, each well was inspected microscopically for cell lysis.

Transcription of the transgene (BEV2.BGI2.2VEB) induces post-transcriptional gene silencing of the BEV RNA polymerase gene, necessary for viral replication. Silencing of the BEV RNA polymerase gene induces resistance to infection by the Bovine enterovirus. These cell lines will continue to divide and grow in the presence of the virus, while control cells die within 48 hr. Viral-tolerant cells are used for further analysis.

(d) Generation of CRIB-1 Viral Tolerant Cell Lines

To determine whether cells transformed with pCMV.BEV.EGFP.VEB or pCMV.BEV2.BGI2.2VEB were tolerant to BEV infection, transformed cell lines were challenged with dilutions of BEV and monitored for survival. To overcome inherent variation in these assays, multiple challenges were performed and lines consistently showing viral tolerance were isolated for further examination. Results of these experiments are shown below in Tables 3 and 4.

TABLE 3
CRIB-1 cells transfected with pCMV.BEV.EGFP.VEB
(CRIB-1 EGFP)

| | Challenge 1 | | Challenge 2 | | Challenge 3 | | Challenge 4 | |
|---|---|---|---|---|---|---|---|---|
| Cell line | 10⁻⁴ | 10⁻⁵ | 10⁻⁴ | 10⁻⁵ | 10⁻⁴ | 10⁻⁵ | 10⁻⁴ | 10⁻⁵ |
| CRIB-1 | nd | nd | − | − | − | − | − | − |
| CRIB-1 EGFP | − | − | − | − | − | − | + | − |
| #1 | | | | | | | | |
| CRIB-1 EGFP | − | − | + | ++ | − | − | nd | nd |
| #3 | | | | | | | | |
| CRIB-1 EGFP | − | − | − | − | − | − | ++ | − |
| #4 | | | | | | | | |
| CRIB-1 EGFP | − | − | + | +++ | − | − | nd | nd |
| #5 | | | | | | | | |
| CRIB-1 EGFP | − | + | − | − | − | − | − | − |
| #6 | | | | | | | | |
| CRIB-1 EGFP | + | + | − | + | + | + | nd | nd |
| #7 | | | | | | | | |
| CRIB-1 EGFP | + | +++ | + | + | + | − | ++ | |
| #8 | | | | | | | | |
| CRIB-1 EGFP | − | − | − | + | + | + | nd | nd |
| #9 | | | | | | | | |
| CRIB-1 EGFP | − | + | − | + | + | ++ | nd | nd |
| #10 | | | | | | | | |
| CRIB-1 EGFP | + | ++ | − | − | + | +++ | nd | nd |
| #11 | | | | | | | | |
| CRIB-1 EGFP | − | + | + | ++ | + | + | nd | nd |
| #12 | | | | | | | | |
| CRIB-1 EGFP | − | − | + | + | − | − | nd | nd |
| #13 | | | | | | | | |
| CRIB-1 EGFP | ++ | ++ | + | ++ | ++ | + | + | + |
| #14 | | | | | | | | |
| CRIB-1 EGFP | − | + | ++ | ++ | + | ++ | nd | nd |
| #15 | | | | | | | | |
| CRIB-1 EGFP | − | + | − | ++ | + | ++ | nd | nd |
| #16 | | | | | | | | |
| CRIB-1 EGFP | − | − | + | + | − | − | nd | nd |
| #17 | | | | | | | | |
| CRIB-1 EGFP | + | + | ++ | + | ++ | ++ | nd | nd |
| #18 | | | | | | | | |
| CRIB-1 EGFP | − | − | − | − | + | +++ | nd | nd |
| #20 | | | | | | | | |
| CRIB-1 EGFP | − | ++ | + | ++ | + | + | nd | nd |
| #21 | | | | | | | | |
| CRIB-1 EGFP | − | + | + | + | + | nd | nd | |
| #22 | | | | | | | | |
| CRIB-1 EGFP | − | − | − | ++ | − | ++ | − | − |
| #23 | | | | | | | | |
| CRIB-1 EGFP | − | − | + | ++ | − | + | | |
| #24 | | | | | | | | |
| CRIB-1 EGFP | − | + | − | +++ | − | − | nd | nd |
| #25 | | | | | | | | |
| CRIB-1 EGFP | + | ++ | ++ | +++ | ++ | +++ | − | − |
| #26 | | | | | | | | |

−: no cells surviving
+: 1-10% of cells surviving.
++: 10-90% of cells surviving.
+++: 90%+ of cells surviving
nd: not done.

TABLE 4
CRIB-1 cells transfected with pCMV.BEV2.BGI2.2VEB
(CRIB-1 BGI2)

| | Challenge 1 | | Challenge 2 | | Chalenge 3 | | Challenge 4 | |
|---|---|---|---|---|---|---|---|---|
| Cell | 10⁻⁴ | 10⁻⁵ | 10⁻⁴ | 10⁻⁵ | 10⁻⁴ | 10⁻⁵ | 10⁻⁴ | 10⁻⁵ |
| CRIB-1 | nd | nd | − | − | − | − | − | − |
| CRIB-1 BGI2 | − | − | − | − | − | − | nd | nd |
| #1 | | | | | | | | |
| CRIB-1 BGI2 | − | − | − | + | − | − | − | − |
| #2 | | | | | | | | |
| CRIB-1 BGI2 | − | − | ++ | ++ | + | ++ | nd | nd |
| #3 | | | | | | | | |
| CRIB-1 BGI2 | − | − | − | + | − | − | nd | nd |
| #4 | | | | | | | | |
| CRIB-1 BGI2 | − | − | − | ++ | − | − | nd | nd |
| #5 | | | | | | | | |
| CRIB-1 BGI2 | + | + | +++ | ++ | + | + | nd | nd |
| #6 | | | | | | | | |
| CRIB-1 BGI2 | + | + | − | +++ | − | − | nd | nd |
| #7 | | | | | | | | |
| CRIB-1 BGI2 | − | + | +++ | ++ | − | + | nd | nd |
| #8 | | | | | | | | |
| CRIB-1 BGI2 | − | + | − | ++ | + | ++ | − | ++ |
| #9 | | | | | | | | |
| CRIB-1 BGI2 | ++ | ++ | ++ | +++ | + | + | − | − |
| #10 | | | | | | | | |
| CRIB-1 BGI2 | + | ++ | + | + | − | + | nd | nd |
| #11 | | | | | | | | |
| CRIB-1 BGI2 | + | + | + | +++ | − | − | nd | nd |
| #12 | | | | | | | | |
| CRIB-1 BGI2 | − | − | +++ | +++ | − | − | nd | nd |
| #13 | | | | | | | | |
| CRIB-1 BGI2 | + | ++ | + | ++ | + | nd | nd | |
| #14 | | | | | | | | |
| CRIB-1 BGI2 | + | + | + | ++ | + | ++ | − | − |
| #15 | | | | | | | | |
| CRIB-1 BGI2 | − | − | − | − | − | − | nd | nd |
| #16 | | | | | | | | |
| CRIB-1 BGI2 | − | + | − | ++ | − | − | nd | nd |
| #17 | | | | | | | | |
| CRIB-1 BGI2 | − | − | − | +++ | − | − | nd | nd |
| #18 | | | | | | | | |
| CRIB-1 BGI2 | − | − | − | ++ | + | +++ | + | +++ |
| #19 | | | | | | | | |
| CRIB-1 BGI2 | + | + | + | +++ | + | + | nd | nd |
| #20 | | | | | | | | |
| CRIB-1 BGI2 | − | − | − | − | − | − | − | − |
| #21 | | | | | | | | |
| CRIB-1 BGI2 | − | − | − | − | − | − | − | − |
| #22 | | | | | | | | |
| CRIB-1 BGI2 | − | + | +++ | +++ | + | + | nd | nd |
| #23 | | | | | | | | |
| CRIB-1 BGI2 | − | ++ | +++ | + | − | − | nd | nd |
| #24 | | | | | | | | |

−: no cells surviving
+: 1-10% of cells surviving.
++: 10-90% of cells surviving.
+++: 90%+ of cells surviving
nd: not done.

These data showed that viral-tolerant cell lines could be defined in this fashion In addition, cells which survived this viral challenge could be grown up for further analyses.

To further define the degree of viral tolerance in such cell lines, the cell line CRIB-1 BGT2 #19, and viral-tolerant cells grown from cells that survived the initial challenge (line CRIB-1 BGI2 #19(tol)), were further analyzed using finer scale serial dilutions of BEV. Three-fold serial dilutions of BEV were used to infect cell lines in triplicate using the procedure outlined in Section 3(c). The results of these experiments are shown in Table 5.

TABLE 5

| | Dilution of viral stock | | | | | |
|---|---|---|---|---|---|---|
| Cell line | 3.3 × 10⁻⁴ | 1.1 × 10⁻⁴ | 3.7 × 10⁻⁵ | 1.2 × 10⁻⁵ | 4.1 × 10⁻⁶ | 1.3 × 10⁻⁶ |
| CRIB-1 Replicate 1 | − | − | − | − | − | +++ |
| CRIB-1 Replicate 1 | − | − | − | − | − | + |
| CRIB-1 Replicate 1 | − | − | − | − | − | +++ |
| CRIB-1 BGI2 #19 | − | − | + | + | +++ | |
| Replicate 1 | | | | | | |
| CRIB-1 BGI2 #19 | − | − | − | − | ++ | +++ |
| Replicate 2 | | | | | | |
| CRIB-1 BGI2 #19 | − | − | − | + | +++ | +++ |
| Replicate 3 | | | | | | |
| CRIB-1 BGI2 #19(tol) | − | − | + | + | +++ | +++ |
| Replicate 1 | | | | | | |
| CRIB-1 BGI2 #19(tol) | − | − | + | + | ++ | +++ |
| Replicate 2 | | | | | | |
| CRIB-1 BGI2 #19(tol) | − | − | + | + | +++ | +++ |
| Replicate 3 | | | | | | |

−: no cells surviving 48 hr post-infection
+: 1-10% of cells surviving 48 hr post-infection.
++: 10-90% of cells surviving 48 hr post-infection.
+++: 90%+ of cells surviving 48 hr post-infection.

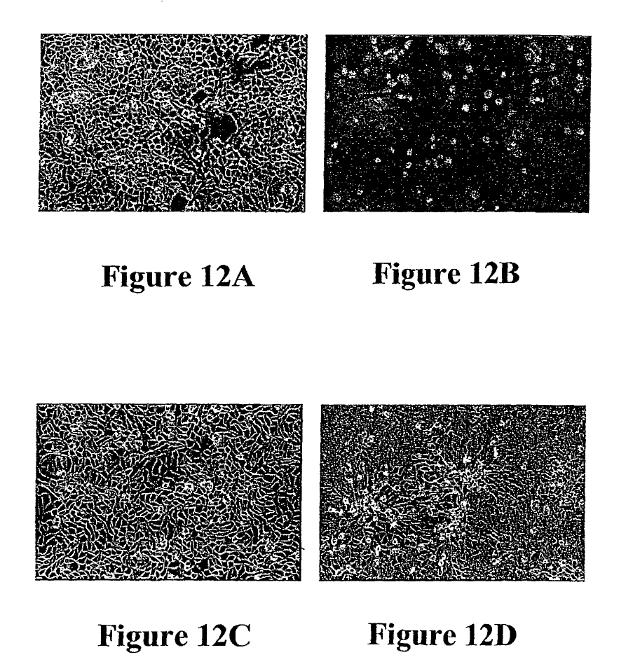

These data showed that the cell lines CRIB-1 BGI2 #19 and CRIB-1 BGI2 #19(tol) were tolerant to higher titres of BEV than the parental CRIB-1 line. FIGS. 12A, 12B and 12C shows micrographs comparing CRIB-1 and CRIB-1 BGI2 #19(tol) cells before and 48 hr after BEV infection.

4. Analysis by Nuclear Transcription Run-On Assays

To detect transcription of the transgene in the nucleus of CRIB-1 cells, nuclear transcription run-on assays are performed on cell-free nuclei isolated from actively dividing cells. The nuclei are obtained according to the cell nuclei isolation protocol set forth in Example 10, above.

Analysis of the nuclear RNA transcript for the transgene BEV2.BGI2.2VEB from the transfected plasmid pCMV.BEV2.BGI2.2VEB is performed according to the nuclear transcription run-on protocol set forth in Example 10, above.

5. Comparison of mRNA in Non-Transformed and Co-Suppressed Lines

Messenger RNA for BEV RNA polymerase and RNA transcribed from the transgene BEV2.BGI2.2VEB are analyzed according to the protocol set forth in Example 10, above.

6. Southern Analysis

Individual transgenic CRIB-1 cell lines are analyzed by Southern blot analysis to confirm integration of the transgene and determine copy number of the transgene. The procedure is carried out according to the protocol set forth in Example 10, above.

### EXAMPLE 14

Co-Suppression of Tyrosinase in Murine Type B16 Cells In Vitro

1. Culturing of Cell Lines

B16 cells derived from murine melanoma (ATCC CRL-6322) were grown as adherent monolayers using RPMI 1640 supplemented with 10% v/v FBS, as described in Example 10, above.

2. Preparation of Genetic Constructs

(a) Interim Plasmid

Plasmid TOPO.TYR

Total RNA was purified from cultured murine B16 melanoma cells and cDNA prepared as described in Example 11.

To amplify a region of the murine tyrosinase gene, 2 μl of this mixture was used as a substrate for PCR amplification using the primers:

TYR-F: GTT TCC AGA TCT CTG ATG GC [SEQ ID NO: 9]

and

TYR-R: AGT CCA CTC TGG ATC CTA GG [SEQ ID NO: 10].

The PCR amplification was performed using HotStarTaq DNA polymerase according to the manufacturer's protocol (Qiagen). PCR amplification conditions involved an initial activation step at 95° C. for 15 mins, followed by 35 amplification cycles of 94° C. for 30 secs, 55° C. for 30 secs and 72° C for 60 secs, with a final elongation step at 72° C. for 4 mins.

The PCR amplified region of tyrosinase was column purified (PCR purification column, Qiagen) and then cloned into pCR (registered trademark) 2.1-TOPO according to the manufacturer's instructions (Invitrogen) to make plasmid TOPO.TYR.

(b) Test Plasmids

Plasmid pCMV.EGFP

Plasmid pCMV.EGFP (FIG. 5) is capable of expressing the entire EGFP open reading frame and is used in this and subsequent examples as a positive transfection control (refer to Example 12, 2(b)).

Plasmid PCMV.TYR.BGI2.RYT

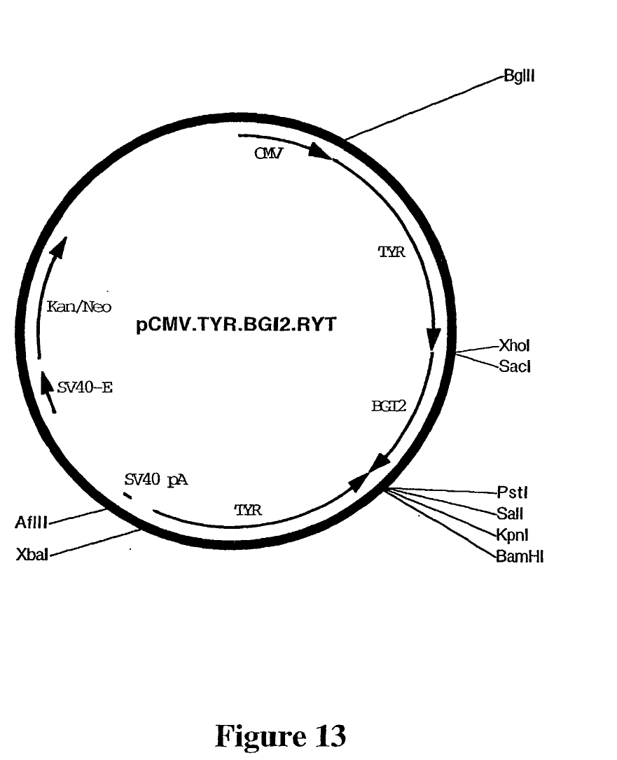

Plasmid pCMV.TYR.BGI2.RYT (FIG. 13) contains an inverted repeat, or palindrome, of a region of the murine tyrosinase gene that is interrupted by the insertion of the human β-globin intron 2 sequence therein. Plasmid pCMV.TYR.BGI2.RYT was constructed in successive steps: (i) the TYR sequence from plasmid TOPO.TYR was sub-cloned in the sense orientation as a BglII-to-BamHI fragment into BglII-digested pCMV.BGI2 to make plasmid pCMV.TYR.BGI2, and (ii) the TYR sequence from plasmid TOPO.TYR was sub-cloned in the antisense orientation as a BglII-to-BamHI fragment into BamHI-digested pCMV.TYR.BGI2 to make plasmid pCMV.TYR.BGI2.RYT.

Plasmid pCMV.TYR

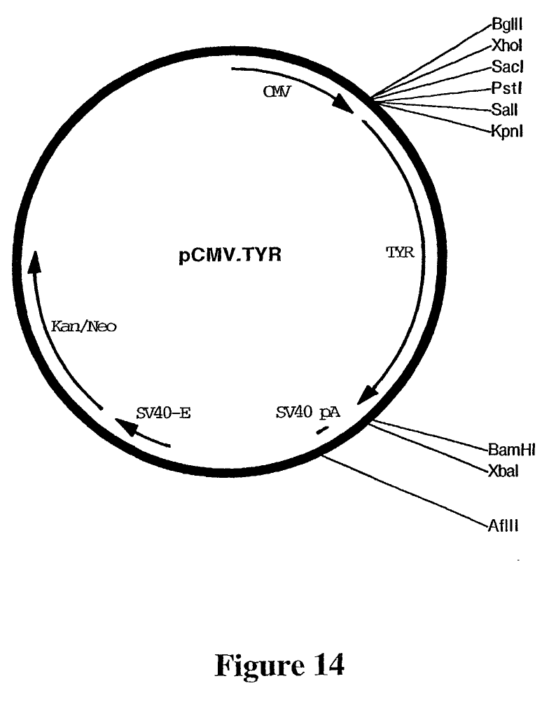

Plasmid pCMV.TYR (FIG. 14) contains a single copy of mouse tyrosinase cDNA sequence, expression of which is driven by the CMV promoter. Plasmid pCMV.TYR was constructed by cloning the TYR sequence from plasmid TOPO.TYR as a BamHI-to-BglII fragment into BamHI-digested pCMV.cass and selecting plasmids containing the TYR sequence in a sense orientation relative to the CMV promoter.

Plasmid pCMV.TYR.TYR

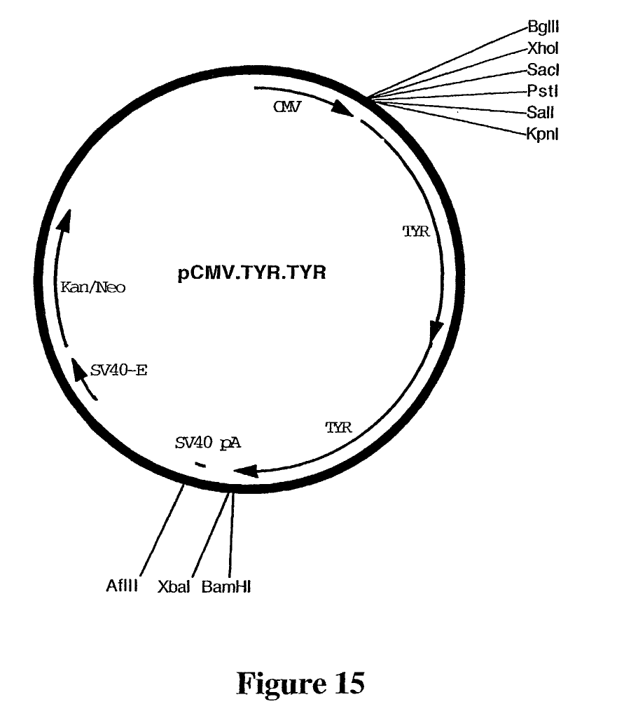

Plasmid pCMV.TYR.TYR (FIG. 15) contains a direct repeat of the mouse tyrosinase cDNA sequence, expression of which is driven by the CMV promoter. Plasmid pCMV.TYR.TYR was constructed by cloning the TYR sequence from plasmid TOPO.TYR as a BamHI-to-BglII fragment into BamHI-digested pCMV.TYR and selecting plasmids containing the second TYR sequence in a sense orientation relative to the CMV promoter.

3. Detection of Co-Suppression Phenotype

(a) Reduction of Melanin Pigmentation through PTGS of Tyrosinase by Insertion of a Region of the Tyrosinase Gene into Murine Melanoma B16 Cells

Tyrosinase is the major enzyme controlling pigmentation in mammals. If the gene is inactivated, melanin will no longer be produced by the pigmented B16 melanoma cells. This is essentially the same process that occurs in albino animals.

Transformations were performed in 6 well tissue culture vessels. Individual wells were seeded with 1×10⁵ cells in 2 ml of RPMI 1640, 10% v/v FBS and incubated at 37° C., 5% V/V CO₂ until the monolayer was 60-90% confluent, typically 16 to 24 hr.

Subsequent procedures were as described above in Example 13, 3(a), except that B16 cells were incubated with the DNA liposome complexes at 37° C. and 5% v/v CO₂ for 3 to 4 hr only.

Individual colonies of stably transfected B16 cells were cloned, maintained and stored as described in Example 10, above.

Thirty six clones stably transformed with pCMV.TYR.BGI2.RYT, 34 clones stably transformed with pCMV.TYR and 37 clones stably transformed with pCMV.TYR.TYR were selected for subsequent analyses.

When the endogenous tyrosinase gene is post-transcriptionally silenced, melanin production in the B16 cells is reduced. B16 cells that would normally appear to contain a dark brown pigment will now appear lightly pigmented or unpigmented.

(b) Visual Monitoring of Melanin Production in Transformed B16 Cell Lines

To monitor melanin content of transformed cell lines, cells were trypsinized and transferred to media containing PBS to inhibit trypsin activity. Cells were then counted with a haemocytometer and 2×10⁶ cells transferred to a microfuge tube. Cells were collected by centrifugation at 2,500 rpm for 3 min at room temperature and pellets examined visually.

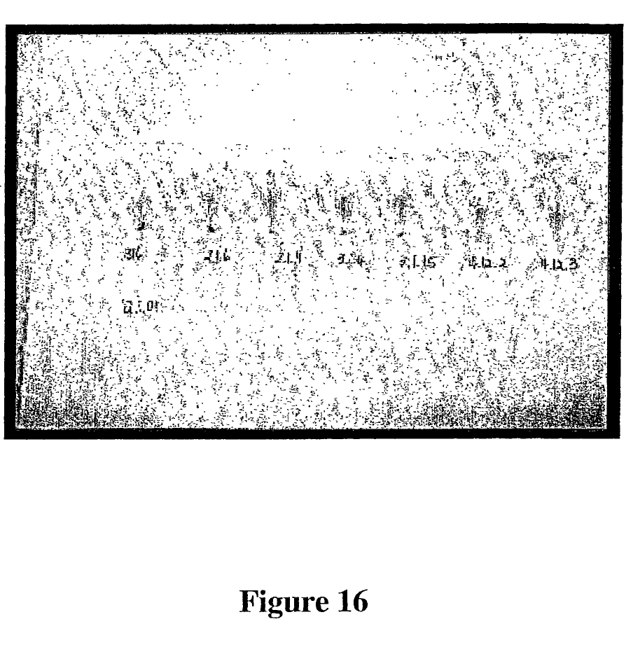

Five clones transformed with pCMV.TYR.BGI2.RYT, namely B 16.2 1.11, B16 3.1.4, B16 3.1.15, B16 4.12.2 and B16 4.12.3, were considerably paler than the B16 controls (FIG. 16). Four clones transformed with pCMV.TYR (B16+Tyr 2.3, B16+Tyr 2.9, B16+Tyr 3.3, B16+Tyr 3.7 and B16+Tyr 4.10) and five clones transformed with pCMV.TYR.TYR (B16+TyrTyr 1.1, B16+TyrTyr 2.9, B16+TyrTyr 3.7, B16+TyrTyr 3.13 and B16+TyrTyr 4.4) were also significantly paler than the B16 controls.

(c) Identification of Melanin by Staining According to Schmorl

Specific diagnosis for the presence of cellular melanin can be achieved using a modified Schmorl's melanin staining method (Koss, L. G. (1979). Diagntostic Cytology. J. B. Lippincott, Philadelphia). Using this method, the presence of melanin in the cell is detected by a specific staining procedure that converts melanin to a greenish-black pigment.

Cell populations to be stained were resuspended at a concentration of 500,000 cells per ml in RPMI 1640 medium. Volumes of 200 μl were dropped onto surface-sterilized microscope slides and slides were incubated at 37° C. in a humidified atmosphere in 100 mm TC dishes until cells had adhered firmly. The medium was removed and cells were fixed by air drying on a heating block at 37° C. for 30 min then post-fixed with 4% w/v paraformaldehyde (Sigma) in PBS for 1 hr. Fixed cells were hydrated by dipping in 96% v/v ethanol in distilled water, 70% v/v ethanol, 50% v/v ethanol then distilled water. Slides with adherent cells were left for 1 hr in a ferrous sulfate solution [2.5% w/v ferrous sulfate in water] then rinsed in four changes of distilled water, 1 min each. Slides were left for 30 min in a solution of potassium ferricyanide [1% (w/v) potassium ferricyanide in 1 ((v/v) acetic acid in distilled water]. Slides were dipped in 1% v/v acetic acid (15 dips) then dipped in distilled water (15 dips).

Cells were stained for 1-2 min in a Nuclear Fast Red preparation [0.1% w/v Nuclear Fast Red (C.I. 60760 Sigma N 8002) dissolved with heating in 5% w/v ammonium sulfate in water]. Fixed and stained cells on slides were washed by dipping in distilled water (15 dips). Cover slips were mounted on slides in glycerol/DABCO [25 mg/ml DABCO (1,4 diazabicyclo(2.2.2)octane (Sigma D 2522)) in 80 % v/v glycerol in PBS]. Cells were examined by bright field microscopy using a 100×oil immersion objective.

The results of staining with Schmorl's stain correlated with the simple visual data illustrated in FIG. 16 for all cell lines. When B16 cells were stained with the above procedure, melanin was obvious in most cells. In contrast, fewer cells stained for melanin in the transformed lines B16 2.1.11, B116 3.1.4, B16 3.1.15, B16 4.12.2, B16 4.12.3, B16 Tyr 2.3, B16 Tyr 2.9, B16 Tyr 4.10, B16 TyrTyr 1.1, B16 TyrTyr 2.9 and B16 TyrTyr 3.7, consistent with the reduced total tyrosinase activity observed in these cell lines.

(d) Assaying Tyrosinase Enzyme Activity in Transformed Cell Lines

Tyrosinase catalyzes the first two steps of melanin synthesis: the hydroxylation of tyrosine to dopa (dihydroxyphenylalanine) and the oxidation of dopa to dopaquinone. Tyrosinase can be measured as its dopa oxidase activity. This assay uses Besthorn's hydrazone (3-methyl-2-benzothiazolinonehydrazone hydrochloride, MBTH) to trap dopaquinone formed by the oxidation of L-dopa. Presence of a low concentration of N,N′-dimethylformamide in the assay mixture renders the MBTH soluble and the method can be used over a range of pH values. MBTH reacts with dopaquinone by a Michael addition reaction and forms a dark pink product whose presence is monitored using a spectrophotometer or plate reader. It is assumed that the reaction of the MBTH with dopaquinone is very rapid relative to the enzyme-catalyzed oxidation of 1-dopa. The rate of production of the pink pigment can be used as a quantitative measure of enzyme activity (Winder and Harris, 1991; Dutkiewicz et al., 2000).

B16 cells and transformed B16 cell lines were plated into individual wells of a 96-well plate in triplicate. Constant numbers of cells (25,000) were transferred into individual wells and cells were incubated overnight. Tyrosinase assays were performed as described below after either 24 or 48 hr incubation.

Individual wells were washed with 200 μl PBS and 20 μl of 0.5% v/v Triton X-100 in 50 mM sodium phosphate buffer (pH 6.9) was added to each well. Cell lysis and solubilisation was achieved by freeze-thawing plates at −70° C. for 30 min, followed by incubating at room temperature for 25 min and 37° C. for 5 min.

Tyrosinase activity was assayed by adding 190 μl freshly-prepared assay buffer (6.3 mM MBTH, 1.1 mM L-dopa, 4% v/v N,N′-dimethylformamide in 48 mM sodium phosphate buffer (pH 7.1)) to each well. Colour formation was monitored at 505 nm in a Tecan plate reader and data collected using X/Scan Software. Readings were taken at constant time intervals and reactions monitored at room temperature, typically 22° C. Results were calculated as the average of enzyme activities as measured for the triplicate samples. Data were analyzed and tyrosinase activity estimated at early time-points when product formation was linear, typically between 2 and 12 min. Results from these experiments are shown below in Tables 6 and 7.

TABLE 6

| | Tyrosinase activity | Relative tyrosinase |
|---|---|---|
| | (Δ OD 505 nm/min/ | activity compared to |
| Cell Line | 25,000 cells) | B16 cells (%) |
| B16 | 0.0123 | 100 |
| B16 2.1.6 | 0.0108 | 87.8 |
| B16 2.1.11 | 0.0007 | 5.7 |
| B16 3.1.4 | 0.0033 | 26.8 |
| B16 3.1.15 | 0.0011 | 8.9 |
| B16 4.12.2 | 0.0013 | 10.6 |
| B16 4.12.3 | 0.0011 | 8.9 |
| B16 Tyr Tyr 1.1 | 0.0043 | 34 |
| B16 Tyr Tyr 2.9 | 0.0042 | 34.1 |
| B16 Tyr Tyr 3.7 | 0.0087 | 70.7 |

TABLE 7

| | Tyrosinase activity | Relative tyrosinase |
|---|---|---|
| | (Δ OD 505 nm/min/ | activity compared to |
| Cell Line | 25,000 cells) | B16 cells (%) |
| B16 | 0.0200 | 100 |
| B16 Tyr 2.3 | 0.0036 | 18.2 |
| B16 Tyr 2.9 | 0.0017 | 8.7 |
| B16 Tyr 4.10 | 0.0034 | 17.2 |

These data showed that tyrosinase enzyme activity was inhibited in lines transformed with the constructs pCMV.TYR.BGI2.RYT, pCMV.TYR and pCMV.TYR.TYR

4. Analysis by Nuclear Transcription Run-On Assays

To detect transcription of the transgene RNAs in the nucleus of B16 cells, nuclear transcription run-on assays were performed on nuclei isolated from actively dividing cells. The nuclei were obtained according to the cell nuclei isolations protocol set forth in Example 10, above.

Analysis of the nuclear RNA transcripts for the transgene TYR.BGI2.RYT from the transfected plasmid pCMV.TYR.BGI2.RYT and the endogenous tyrosinase gene is performed according to the nuclear transcription run-on protocol set forth in Example 10, above.

To estimate transcription rates of the endogenous tyrosinase gene in B16 cells and the transformed lines B16 3.1.4 and B16 Tyr Tyr 1.1, nuclear transcription run-on assays were performed on nuclei isolated from actively dividing cells. The nuclei were obtained according to the cell nuclei isolation protocol set forth in Example 10, above, and run-on transcripts were labelled with biotin and purified using streptavidin capture as outlined in Example 10.

To determine the transcription rate of the endogenous tyrosinase gene in the above cell lines, the amount of biotin-labelled tyrosinase transcripts isolated from nuclear run-on assays was quantified using real time PCR reactions. The relative transcription rates of the endogenous tyrosinase gene were estimated by comparing the levels of biotin-labelled tyrosinase RNA to the levels of a ubiquitously-expressed endogenous transcript, namely murine glyceraldehyde phosphate dehydrogenase (GAPDH).

The levels of expression of both the endogenous tyrosinase and mouse GAPDH genes were determined in duplex PCR reactions. To permit quantitative interpretation of these data, a standard curve was generated using oligo dT-purified RNA isolated from B16 cells. Oligo dT-purification was achieved using Dynabeads mRNA DIRECT Micro Kit according to the manufacturer's instructions (Dynal). Results from these analyses are shown in Table 8.

TABLE 8

| | Tyrosinase | | | |
|---|---|---|---|---|
| | and GAPDH RNA levels | | | |
| | in biotin-captured nuclear | | | Relative |
| | transcription run-on RNAs | | | transcription rate |
| Cell Line | Cₜ TYR | Cₜ GAPDH | Δ Cₜ | of Tyrosinase gene |
| B16 | 38.6 | 27.2 | 11.5 | 1.00 |
| B16 3.1.4 | 36.5 | 24.4 | 12.1 | 0.65 |
| B16 Tyr Tyr 1.1 | 38.5 | 26.2 | 12.4 | 0.59 |

These data show clearly that rates of transcription from the endogenous tyrosinase gene in the nuclei of the two silenced B16 cell lines B16 3.1.4 and B16 TyrTyr 1.1, transformed with pCMV.TYR.BGI2.RYT and pCMV.TYR.TYR, respectively, are not significantly different from the rate of transcription from the tyrosinase gene in nuclei of non-transformed B16 cells.

5. Comparison of mRNA in Non-Transformed and Co-Suppressed Lines

Messenger RNA for endogenous tyrosinase and RNA transcribed from the transgene TYR.BGI2.RYT are analyzed according to the protocols set forth in Example 10, above.

To obtain accurate estimates of tyrosinase mRNA levels in B16 and transformed lines, real time PCR reactions were employed. Results from these analyses are shown in Table 9.

TABLE 9

| | Tyrosinase and | | | |
|---|---|---|---|---|
| | GAPDH RNA levels in | | | |
| | oligo-dT purified total RNAs | | | Relative levels of |
| Cell Line | Cₜ TYR | Cₜ GAPDH | Δ Cₜ | tyrosinase mRNA |
| B16 | 33.5 | 21.9 | 11.7 | 1.0 |
| B16 3.1.4 | 33.8 | 22.1 | 11.7 | 1.0 |
| B16 Tyr Tyr 1.1 | 35.1 | 23.0 | 12.1 | 0.7 |

These data show clearly that the level of tyrosinase mRNA (as poly(A)RNA) in the two silenced B16 cell lines B16 3.1.4 and B16 TyrTyr 1.1, transformed with pCMV.TYR.BGI2.RYT and pCMV.TYR.TYR, respectively, are not significantly different from the level of tyrosinase mRNA in non-transformed B16 cells.

6. Southern Analysis

Individual transgenic B16 cell lines are analyzed by Southern blot analysis to confirm integration and determine copy number of the transgene. The procedure is carried out according to the protocol set forth in Example 10, above.

### EXAMPLE 15

Co-Suppression of Tyrosinase in Mus musculus Strains C57BL/6 and C57BL/6×DBS Hybrid In Vivo

1. Preparation of Constructs

The interim plasmid TOPO.TYR and test plasmid pCMV.TYR.BGI2.RYT were generated as described in Example 14, above.

2. Generation of Transgenic Mice

Transgenic mice were generated through genetic modification of pronuclei of zygotes. After isolation from oviducts, zygotes were placed on an injection microscope and the transgene, in the form of a purified DNA solution, was injected into the most visible pronucleus (U.S. Pat. No. 4,873,191).

Pseudo-pregnant female mice were generated, to act as “recipient mothers”, by induction into a hormonal stage that mimics pregnancy. Injected zygotes were then either cultured overnight in order to assess their viability, or transferred immediately back into the oviducts of pseudo-pregnant recipients. Of 421 injected zygotes, 255 were transferred. Transgenic off-spring resulting from these injections are called “founders”. To determine that the transgene has integrated into the mouse genome, off-spring are genotyped after weaning. Genotyping was carried out by PCR and/or by Southern blot analysis on genomic DNA purified from a tail biopsy.

Founders are then mated to begin establishing transgenic lines. Founders and their offspring are maintained as separate pedigrees, since each pedigree varies in transgene copy number and/or chromosomal location. Therefore, each transgenic mouse generated by pronuclear injection is the founder of a new strain. If the founder is female, some pups from the first letter are analyzed for transgene transmission.

3. Detection of Co-Suppression Phenotype

Visual read-out of successful transgenic mice is an alteration to coat colour. Skin-cell biopsies are harvested from transgenic mice and cultured as primary cultures of melanocytes by standard methods (Bennett et al., 1989; Spanakis et al., 1992; Sviderskaya et al., 1995).

The biopsy area of adult mice is shaved and the skin surface-sterilized with 70% v/v ethanol then rinsed with PBS. The skin biopsy is removed under sterile conditions. Sampling of skin from newborn mice isis done after sacrifice of the animal, which isis then ished in 70% v/v ethanol and rinsed in PBS. Skin samples are dissected under sterile conditions.

All biopsies are stored in PBS in 6-well plates. To obtain single cell suspensions, PBS is pipetted off and skin samples cut into small pieces (2×5 mm) with two scalpels and incubated in 2×trypsin (5 mg/mi) in PBS at 37° C. for about 1 hr for newborn samples and up to 15 hr in 1×trypsin (2.5 mg/ml) at 4° C. for samples of adult skin (0.5 g in 2.5 ml). This digestion separates epidermis from dermis. Trypsin is replaced with RPMI 1640 medium to stop enzyme activity. The epidermis of each piece is separated with fine forceps (sterile) and isolated epidermal samples are collected and pooled in 1×trypsin in PBS. Single cell suspensions are prepared by pipetting and separated cells are collected in RPMI 1640 medium. Trypsinization of epidermal samples can be repeated. Pooled epidermal cells are concentrated by gentle centrifugation (1000 rpm for 3 min) and resuspended in growth medium [RPM 1640 with 5% v/v FBS, 2 mM L-glutamine, 20 units/ml penicillin, 20 μg/ml streptomycin plus phorbol 12-myristate 13-acetate (PMA) 10 ng/ml (16 nM) and cholera toxin (CTX) 20 ng/ml (1.8 nM)]. Suspensions are transferred to T25 flasks and incubated without disturbance for 48 hr. Medium is changed and unattached cells removed at 48 hr. After a further 48-72 hr incubation, the medium is discarded, the attached cells ished with PBS and treated with 1×trypsin in PBS. Melanocytes become preferentially detached after this treatment and the detached cells are transferred to fresh medium in new flasks.

Melanocytes in tissue culture are easily distinguishable from keratinocytes by their morphology. Keratinocytes have a round or polygonal shape; melanocytes appear bipolar or polydendritic. Melanocytes may be stained by Schmorl's method (see Example 14, above) to detect melanin granules. In addition, samples of cultures grown on cover slips are investigated by immunofluorescence labelling (see Example 10, above) with a primary murine monoclonal antibody against MART-1 (NeoMarkers MS-614) which is an antigen found in melanosomes. This antibody does not cross-react with cells of epithelial, lymphoid or mesenchymal origin.

4. Analysis by Nuclear Transcription Run-On Assays

To detect transcription of the tyrosinase endogenous gene and transgene RNAs in the nucleus of primary culture melanocytes, nuclear transcription run-on assays are performed on cell-free nuclei isolated from actively dividing cells, according to the cell nuclei isolation protocol set forth in Example 10, above.

Analysis of nuclear RNA transcripts for the tyrosinase endogenous gene and the transgene from the transfected plasmid pCMV.TYR.BGI2.RYT are performed according to the nuclear transcription run-on protocol set forth in Example 10, above.

5. Comparison of mRNA in Non-Transformed and Co-Suppressed Lines

Messenger RNA for endogenous tyrosinase and RNA transcribed from the transgene TYR.BGI2.RYT are analyzed according to the protocols set forth in Example 10, above.

6. Southern Analysis

Primary culture melanocytes are analyzed by Southern blot analysis to confirm integration and determine copy number of the transgene. This is carried out according to the protocol set forth in Example 10, above.

### EXAMPLE 16

Co-Suppression of α-1,3,-galactosyl Transferase (GalT) in Mus musculus Strain C57BL/6 In Vivo

1. Preparation of Genetic Constructs

(a) Plasmid TOPO.GALT

Total RNA was purified from cultured murine 2.3D17 neural cells and cDNA prepared as described in Example 11.

To amplify the 3α-UTR of the murine α-1,3,-galactosyl transferase (GalT) gene, 2 μl of this mixture was used as a substrate for PCR amplification using the primers:

GALT-F2: CAC AGA CAG ATC TCT TCA GG [SEQ ID NO:11]

and

GALT-R1: ACT TTA GAC GGA TCC AGC AC [SEQ ID NO: 12].

The PCR amplification was performed using HotStarTaq DNA polymerase according to the manufacturer's protocol (Qiagen). PCR amplification conditions involved an initial activation step at 95° C. for 15 mins, followed by 35 amplification cycles of 94° C. for 30 secs, 55° C. for 30 secs and 72° C. for 60 secs, with a final elongation step at 72° C. for 4 mins.

The PCR amplified region of GalT was column purified (PCR purification column, Qiagen) and then cloned into pCR2.1-TOPO according to the manufacturer's instructions (nvitrogen), to make plasmid TOPO.GALT.

(b) Test Plasmid

Plasmid PCMV.GALTBGI2.TLAG

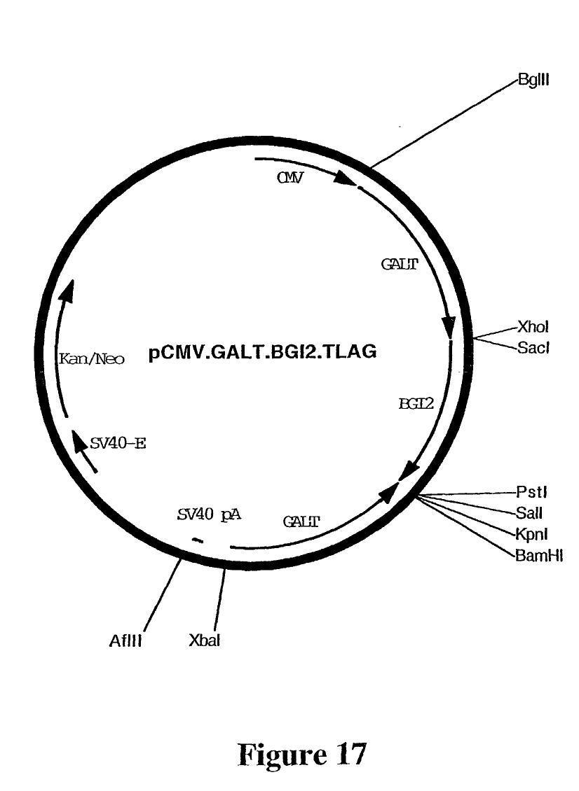

Plasmid pCMV.GALT.BGI2.TLAG (FIG. 17) contains an inverted repeat, or palindrome, of a region of the Murine 3′UTR GalT gene that is interrupted by the insertion of the human β-globin intron 2 sequence therein. Plasmid pCMV.GALT.BGI2.TLAG was constructed in successive steps: (i) the GALT sequence from plasmid TOPO.GALT was sub-cloned in the sense orientation as a BglII-to-BamHI fragment into BglII-digested pCMV.BGI2 to make plasmid pCMV.GALT.BGI2, and (ii) the GALT sequence from plasmid TOPO.GALT was sub-cloned in the antisense orientation as a BglII-to-BamHI fragment into BamHI-digested pCMV.GALT.BGI2 to make plasmid pCMV.GALT.BGI2.TLAG.

2. Generation of Transgenic Mice

Transgenic mice were generated through genetic modification of pronuclei of zygotes. After isolation from oviducts, zygotes were placed on an injection microscope and the transgene, in the form of a purified DNA solution, was injected into the most visible pronucleus (U.S. Pat. No. 4,873,191).

Pseudo-pregnant female mice were generated, to act as “recipient mothers”, by induction into a hormonal stage that mimics pregnancy. Injected zygotes were then either cultured overnight in order to assess their viability, or transferred imnmediately back into the oviduct of pseudo-pregnant recipients. Of 99 injected zygotes, 25 were transferred. Transgenic off-spring resulting from these injections are called “founders”. To determine that the transgene has integrated into the mouse genome, off-spring are genotyped after weaning. Genotyping was carried out by PCR and/or by Southern blot analysis on genomic DNA purified from a tail biopsy.

Founders are then mated to begin establishing transgenic lines. Founders and their offspring are maintained as separate pedigrees, since each pedigree varies in transgene copy number and/or chromosomal location. Therefore, each transgenic mouse generated by pronuclear injection is the founder of a new strain. If the founder is female, some pups from the first letter are analyzed for transgene transmission.

3. Detection of Co-Suppression Phenotype

The enzyme α-1,3,-galactosyl transferase (GalT) catalyzes the addition of galactosyl sugar residues to cell surface proteins in cells of all mammals except humans and other primates. The epitope enabled by the action of GalT is the predominant antigen responsible for the rejection of xenotransplants in humans. Cytological analyses of GalT expression levels in peripheral blood leukocytes (PBL) and splenocytes using FACS confirms the down regulation of the gene's activity.

Analysis of Peripheral Blood Leukoqytes and SplenoCytes from Transgenic Mice by FACS

To analyze cells from transgenic mice transformed with the GalT construct, FACS assays on peripheral blood leukocytes (PBL) and splenocytes are undertaken. White blood cells are the most convenient source of tissue for analysis and these can be isolated from either PBL or splenocytes. To isolate PBL, mice are bled from an eye and 50 to 100 μl of blood collected into heparinized tubes. The red blood cells (RBCs) are lysed by treatment with NH₄Cl buffer (0.168M) to recover the PBLs.

To obtain splenocytes, animals are euthanased, the spleens removed and macerated and RBCs lysed as above. The generated splenocytes are cultured in vitro in the presence of interleukin-2 (IL2; Sigma) to generate short term T cell cultures. The cells are then fixed in 4% w/v PFA in PBS. All steps are performed on ice. GalT activity can be most conveniently assayed using a plant lectin (IB4; Sigma), which binds specifically to galactosyl residues on cell surface proteins. GalT is detected on the cell surface by binding IB4 conjugated to biotin. The leukocytes are then treated with streptavidin conjugated to Cy5 fluorophore. Another cell marker, the T cell specific glycoprotein Thy-1, is labelled with a fluorescein isothiocyanate-conjugated antibody (FITC; Sigma). The leukocytes are incubated in a mixture of the reagents for 30 min to label the cells. After washing, the cells are analyzed on the FACScan. (Tearle, R. G. et al., 1996).

4. Analysis by Nuclear Transcription Run-On Assays

To detect transcription of transgene RNAs in the nucleus of splenocytes, nuclear transcription run-on assays are performed on cell-free nuclei isolated from actively dividing cells. In vitro culturing of splenocytes in the presence of IL-2 generates short term T cell cultures. The nuclei are obtained according to the cell nuclei isolation protocol for suspension cell cultures, set forth in Example 10 above.

Analysis of nuclear RNA transcripts for the GalT endogenous gene and the transgene from the transfected plasmid pCMV.GALT.BGI2.TLAG is performed according to the nuclear transcription run-on protocol set forth in Example 10, above.

5. Comparison of mRNA in Non-Transformed and Co-Suppressed Lines

Messenger RNA for endogenous GalT and RNA transcribed from the transgene GALT.BGI2.TLAG are analyzed according to the protocols set forth in Example 10, above.

6. Southern Analysis

Individual transgenic splenocyte cell lines are analyzed by Southern blot analysis to confirm integration and determine copy number of the transgenes. This is carried out according to the protocol set forth in Example 10, above.

### EXAMPLE 17

Co-Suppression of Mouse Thymidine Kinase in NIH/3T3 Cells In Vitro

Cells produce ribonucleotides and deoxyribonucleotides via two pathways—de novo synthesis or salvage synthesis. De novo synthesis is the assembly of nucleotides from simple compounds such as amino acids, sugars, CO₂ and NH₃. The precursors of purine and pyrimidine nucleotides, inosine 5′-monophosphate (IMP) and uridine 5′-monophosphate (UMP) respectively, are produced first by this pathway. De novo synthesis of IMP and thymridine 5′-monophosphate (TMP) requires tetrahydrofolate derivatives as co-factors and de novo synthesis of these nucleotides is blocked by the antifolate aminopterin which inhibits dihydrofolate reductase. Salvage synthesis refers to enzymatic reactions that convert free preformed purine bases or thymidine to their corresponding nucleotide monophosphates (NMP). When de novo synthesis is blocked, salvage enzymes enable the cell to survive while pre-formed bases are present in the medium.

Mammalian cells normally express several salvage enzymes including thymidine kinase (TK) which converts thymidine to TMP. The drug 5-bromo-2′-deoxyuridine (BrdU; Sigma) selects cells that lack TK. In cells with functioning TK, the enzyme converts the drug analogue to its corresponding 5′-monophosphate which is lethal when incorporated into DNA. Conversely, cells lacking TK expression are unable to grow in HAT medium (Life Technologies) which contains both aminopterin and thymidine. The first factor in the supplement blocks de novo synthesis of NMPs and the second provides a substrate for the TK salvage pathway so that cells with that pathway intact are able to survive.

1. Culturing of T3 Cell Lines

Cells of the murine fibroblast-like line NIH/3T3 (ATCC CRL-1658) were grown as adherent monolayers in DMEM, supplemented with 10% v/v FBS and 2 mM L-glutamine as described in Example 10, above. Cells were routinely grown in incubators at 37° C. in an atmosphere containing 5% v/v CO₂.

2. Preparation of Genetic Constructs

(a) Interim Plasmid

Plasmid TOPO.MTK

A region of the murine thymidine kinase gene was amplified by PCR using murine cDNA as a template. The cDNA was prepared from total RNA isolated from the murine melanoma ine, B16. Total RNA was purified as described in Example 14, above. Murine thymidine kinase sequences were amplified using the primers:—

MTK1: AGA TCT ATT TTT CCA CCC ACG GAC TCT CGG [SEQ ID NO: 13]

and

MTK4: GGA TCC GCC ACG AAC AAG GAA GAA ACT AGC [SEQ ID NO: 14].

The amplification product was cloned into pCR (registered trademark) 2.1-TOPO to create the intermediate clone TOPO.MTK.

(b) Test Plasmid

Plasmid pCM MTKBGI2.KTM

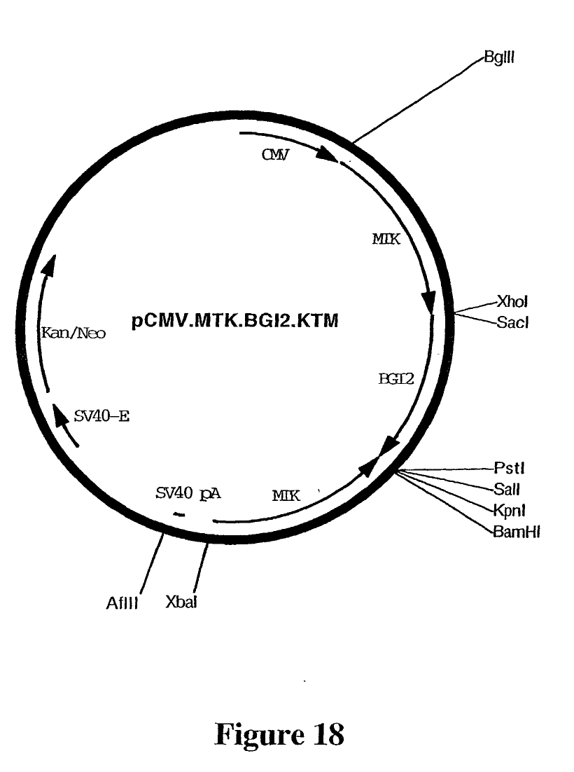

Plasmid pCMV.MTK.BGI2.KTM (FIG. 18) contains an inverted repeat or palindrome of the murine thymidine kinase coding region that is interrupted by the insertion of the human β-globin intron 2 sequence therein. Plasmid pCMV.MTIKBGI2.KTM was constructed in successive steps: (i) the MTK sequence from plasmid TOPO.MTK was sub-cloned in the sense orientation as a BglII-to-BamHI fragment into BglII-digested pCMV.BGI2.cass (Example 11) to make plasmid pCMV.MTK-BGI2, and (ii) the MTK sequence from plasmid TOPO.MTK was sub-cloned in the antisense orientation as a BglII-to-BamHI fragment into BamHI-digested pCMVXTK.BGI2 to make plasmid pCMV.MTK-BGI2.KTM.

3. Detection of Co-Suppression Phenotype

(a) Insertion of TK-Expressing Transgene into NIH/3T3 Cells

Transformations were performed in swell tissue culture vessels. Individual wells were seeded with 1×10⁵ cells in 2 ml of DMEM, 10% v/v FBS and incubated at 37° C., 5% v/v CO₂ until the monolayer was 60-90% confluent, typically 16 to 24 hr.

Subsequent procedures were as described above in Example 13, 3(a), except that NIH/3T3 cells were incubated with the DNA liposome complexes at 37° C. and 5% v/v CO₂ for 3 to 4 hr only.

(b) Post-Transcriptional Silencing of the Mouse TK Gene in NIH/3T3 Cells

NIH/3T3 cells with PTGS of TK are able to tolerate addition of BrdU (NeoMarkers) to their normal growth medium at levels of 100 μg/ml and continue to replicate under this regime. Populations of similarly treated control NIH/3T3 cells cease to replicate and cell numbers do not increase after culture for seven days in BrdU-containing medium. Control NIH/3T3 cells are able to replicate in growth medium containing 1×HAT supplement, while cells with PTGS of TK are unable to grow under these conditions. Further evidence of PTGS of TK is obtained by monitoring incorporation of BrdU in the nucleus via immunofluorescence staining (see Example 10, above) of the cell using a monoclonal antibody directed against BrdU. Clones that fulfil all criteria—(i) resistance to the lethal effects of BrdU; (ii) loss of the nucleotide salvage pathway, and (iii) lack of incorporation of BrdU in the nucleus—undergo direct testing of PTGS via nuclear transcription run-on assays.

4. Analysis by Nuclear Transcription Run-On Assays

To detect transcription of the transgene RNA in the nucleus of NIH/3T3 cells, nuclear transcription run-on assays are performed on cell-free nuclei isolated from actively dividing cells. The nuclei are obtained according to the cell nuclei isolation protocol set forth in Example 10, above.

Analysis of the nuclear RNA transcripts for the transgene MTK-BGI2.KTM from the transfected plasmid pCMV.MTK.BGI2KTM and the endogenous TK gene is performed according to the nuclear transcription run-on protocol set forth in Example 10, above.

5. Comparison of mRNA in Non-Transformed and Co-Suppressed Lines

Messenger RNA for endogenous TK and RNA transcribed from the transgene MTK.BGI2.KTM are analyzed according to the protocols set forth in Example 10, above.

6. Southern Analysis

Individual transgenic NIH/3T3 cell lines are analyzed by Southern blot analysis to confirm integration and determine copy number of the transgene. The procedure is carried out according to the protocol set forth in Example 10, above.

EXAMPLE 18

### Co-Suppression of HER-2 in MDA-MB-468 Cells in Vitro

HER-2 (also designated neu and erbB-2) encodes a 185 kDa transmembrane receptor tyrosine kinase that is constitutively activated at low levels and displays potent oncogenic activity when over-expressed. HER-2 protein over-expression occurs in about 30% of invasive human breast cancers. The biological function of HER-2 is not well understood. It shares a common structural organisation with other members of the epidermal growth factor receptor family and may participate in similar signal transduction pathways leading to changes in cytoskeleton reorganisation, cell motility, protease expression and cell adhesion. Over-expression of HER-2 in breast cancer cells leads to increased tumorigenicity, invasiveness and metastatic potential (Slamon et al., 1987).

1. Culturing of Cell Lines

Human MDA-MB-468 cells were cultured in RPMI 1640 supplemented with 10% v/v FBS. Cells were passaged twice a week by treating with trypsin to release cells and transferring a proportion of the culture to fresh medium, as described in Example 10, above.

2. Preparation of Genetic Constructs

(a) Interim Plasmid

Plasmid TOPO.HER-2

A region of the human HER-2 gene was amplified by PCR using human cDNA as a template. The cDNA was prepared from total RNA isolated from a human breast tumour line, SK-BR-3. Total RNA was purified as described in Example 14, above. Human HER-2 sequences were amplified using the primers:—

H1: CTC GAG AAG TGT GCA CCG GCA CAG ACA TG [SEQ ID NO: 15]

and

H3: GTC GAC TGT GTT CCA TCC TCT GCT GTC AC [SEQ ID NO: 16].

The amplification product was cloned into pCR (registered trademark) 2.1-TOPO to create the intermediate clone TOPO.HER-2.

(b) Test Plasmid

Plasmid pCMV.HER2.BGI2.2REH

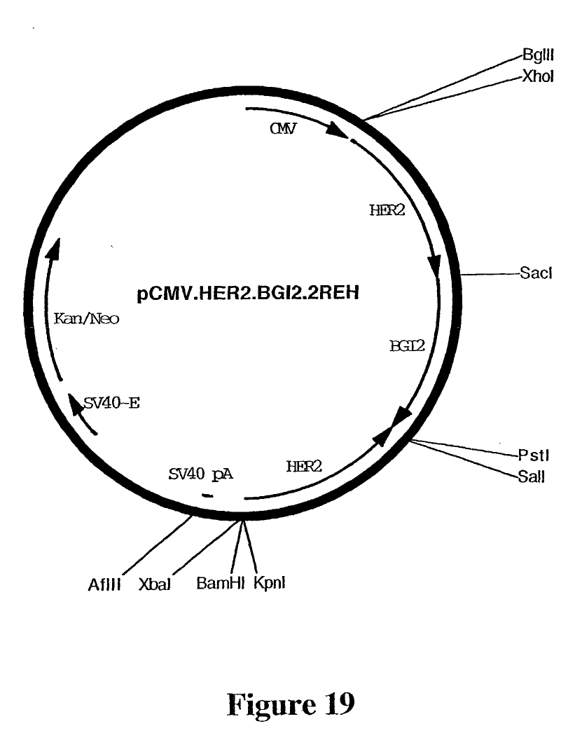

Plasmid pCMV.HER2.BGI2.2REH (FIG. 19) contains an inverted repeat or palindrome of the HER-2 coding region that is interrupted by the insertion of the human β-globin intron 2 sequence therein. Plasmid pCMV.HER2.BGI2.2REH was constructed in successive steps: (i) the HER-2 sequence from plasmid TOPO.HER2 was sub-cloned in the sense orientation as a SalI/XhoI fragment into SalI-digested pCMV.BGI2.cass (Example 11) to make plasmid pCMV.HER2.BGI2, and (ii) the HER2 sequence from plasmid TOPO.HER2 was sub-cloned in the antisense orientation as a SalI/XhoI fragment into XhoI-digested pCMV.HER2.BGI2 to make plasmid pCMV.HER2.BGI2.2REH.

3. Determination of On-Set of Co-Suppression

(a) Transfection of HER-2 Constructs

Transformations were performed in 6-well tissue culture vessels. Individual wells were seeded with 4×10⁵ MDA-MB-468 cells in 2 ml of RPMI 1640 medium, 10% v/v FBS and incubated at 37° C., 5% v/v CO₂ until the monolayer was 60-90% confluent, typically 16 to 24 hr.

Subsequent procedures were as described above in Example 13, 3(a), except that MDA-MB-468 cells were incubated with the DNA liposome complexes at 37° C. and 5% v/v CO₂ for 3 to 4 hr only. Thirty-six transformed cell lines were isolated for subsequent analysis.

(b) Post-Transcriptional Silencing of HER-2 in MDA-MB-468 Cells

MDA-MB468 cells over-express HER-2 and PTGS of the gene in geneticin-selected clones derived from this cell line are tested initially by immunofluorescence labelling of clones (see Example 10, above) with a primary murine monoclonal antibody directed against the extracellular domain of HER-2 protein (Transduction Laboratories and NeoMarkers). Comparison of HER-2 protein levels among (i) MDA-MB-468 cells; (ii) clones exhibiting evidence of PTGS of the gene, and (iii) control human cell lines, are undertaken via western blot analysis (see below) with the anti-HER-2 antibody. Clones that fulfil the criterion of absence of expression of HER-2 protein undergo direct testing of PTGS via nuclear transcription run-on assays.

To analyze HER-2 expression in MDA-MB468 cells and transformed lines, cells were examined using immunofluorescent labelling as described in Example 10. The primary antibody was a mouse Anti-erbB2 monoclonal antibody (Transduction Laboratories, Cat. No. E19420, an IgG2b isotype) used at 1/400 dilution; the secondary antibody was Alexa Fluor 488 goat anti-mouse IgG (H+L) conjugate (Molecular Probes, Cat. No. A-11001) used at 1/100 dilution. As a negative control, MDA-M]B468 cells (parental and transformed hines) were probed with Alexa Fluor 488 goat anti-mouse IgG (H+L) conjugate only.

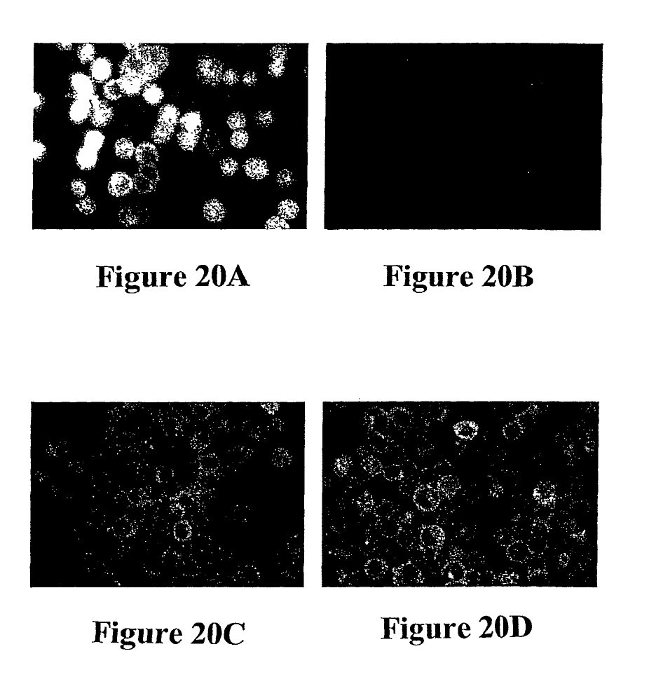

Several MDA-MB-468 cell lines transformed with pCMV.HBR2.BGI2.2REH were found to have reduced immunofluorescence, examples of which are illustrated in FIGS. 20A, 20B, 20C and 20D.

(c) FACS Analysis to Define Cell Lines Showing Reduced Expression of Her-2

To determine the level of expression of HER-2 in transformed cell lines, approximately 500,000 cells grown in a 6-well plate were washed twice with 1×PBS then dissociated with 500 μl cell dissociation solution (Sigma C 5789) according to the manufacturer's instructions (Sigma). Cells were transferred to medium in a microcentrifuge tube and collected by centrifugation at 2,500 rpm for 3 min. The supernatant was removed and cells resuspended in 1 ml 1×PBS.

For fixation, cells were collected by centrifugation as above and suspended in 50 μl PBA (1×PBS, 0.1 % w/v BSA fraction V (Trace) and 0.1 % w/v sodium azide) followed by the addition of 250 μl of 4 % w/v paraformaldehyde in 1×PBS. and incubated at 4° C. for 10, min. To permeabilize cells, cells were collected by centrifligation at 10,000 rpm for 30 sec, the supernatant removed and cells suspended in 50 μl 0.25 % w/v saponin (Sigma S 4521) in PBA and incubated at 4° C. for 10 min. To block cells, cells were collected by centrifugation at 10,000 rpm for 30 sec, the supernatant removed and cells suspended in 50 μl PBA, 1 % v/v FBS and incubated at 4° C. for 10 min.

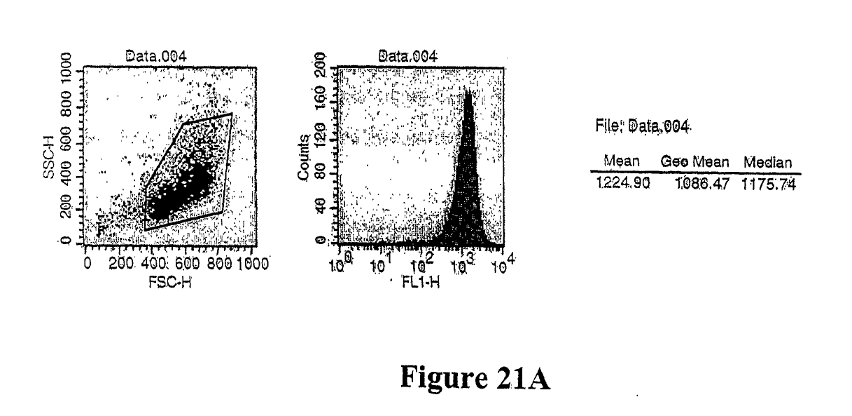

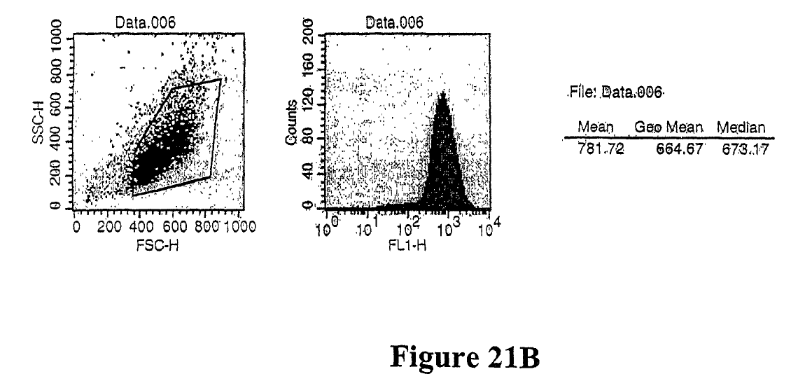

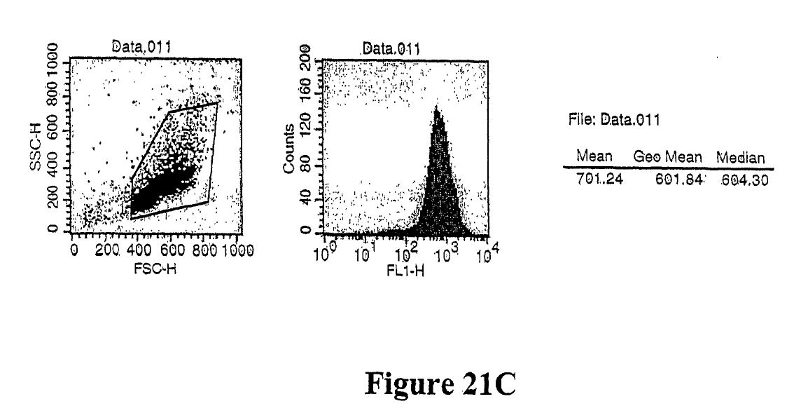

To quantify HER-2 protein, fixed, permeabilized cells were probed with Anti-erbB2 monoclonal antibody (Transduction Laboratories) at 1/100 dilution followed by Alexa Fluor 488 goat anti-mouse IgG conjugate (Molecular Probes) at 1/100 dilution Cells were then analysed by FACS using a Becton Dickinson FACSCalibur and Cellquest software (Becton Dickinson). To estimate true background fluorescence values, unstained MDA-MB-468 cells were probed with an irrelevant prmary antibody (MART-1, an IgG2b antibody (NeoMarkers)) and the Alexa Fluor 488 secondary antibody, both at 1/100 dilutions. Examples of FACS data are shown in FIGS. 21A, 21B and 21C. Results of analyses of all cell lines are compiled in Table 10.

TABLE 10

| | Mean | Geometric mean | Median |
|---|---|---|---|
| Cell line | Fluorescence | Fluorescence | Fluorescence |
| MDA-MB-468 | 5.07 | 4.72 | 4.78 |
| (control.1) | | | |
| MDA-MB-468 | 137.24 | 121.68 | 117.57 |
| (control.2) | | | |
| MDA-MB-468 | 1224.90 | 1086.47 | 1175.74 |
| MDA-MB-468 1.1 | 1167.94 | 1056.17 | 1124.04 |
| MDA-MB-468 1.4 | 781.72 | 664.67 | 673.17 |
| MDA-MB-468 1.5 | 828.34 | 673.82 | 710.50 |
| MDA-MB-468 1.6 | 925.16 | 807.09 | 850.53 |
| MDA-MB-468 1.7 | 870.81 | 749.27 | 791.48 |
| MDA-MB-468 1.8 | 1173.92 | 938.72 | 1124.04 |
| MDA-MB-468 1.10 | 701.24 | 601.84 | 604.30 |
| MDA-MB-468 1.11 | 1103.18 | 980.10 | 1064.99 |
| MDA-MB-468 1.12 | 817.39 | 666.61 | 710.50 |
| MDA-MB-468 2.5 | 966.72 | 862.76 | 905.80 |
| MDA-MB-468 2.6 | 752.70 | 633.49 | 649.38 |
| MDA-MB-468 2.7 | 842.00 | 677.15 | 716.92 |
| MDA-MB-468 2.8 | 986.05 | 792.13 | 881.68 |
| MDA-MB-468 2.9 | 802.36 | 686.06 | 716.92 |
| MDA-MB-468 2.10 | 1061.79 | 944.49 | 1009.04 |
| MDA-MB-468 2.12 | 931.63 | 790.81 | 820.47 |
| MDA-MB-468 2.13 | 894.47 | 792.46 | 827.88 |
| MDA-MB-468 2.15 | 1052.87 | 946.79 | 1009.04 |
| MDA-MB-468 3.1 | 1049.88 | 931.96 | 991.05 |
| MDA-MB-468 3.2 | 897.00 | 802.43 | 842.91 |
| MDA-MB-468 3.4 | 981.63 | 858.95 | 913.98 |
| MDA-MB-468 3.5 | 1072.00 | 930.17 | 982.17 |
| MDA-MB-468 3.7 | 1098.95 | 993.26 | 1036.63 |
| MDA-MB-468 3.8 | 1133.86 | 1026.31 | 1074.61 |
| MDA-MB-468 3.9 | 831.73 | 729.32 | 763.51 |
| MDA-MB-468 3.12 | 1120.82 | 998.67 | 1064.99 |
| MDA-MB-468 3.13 | 1039.41 | 963.71 | 1036.63 |
| MDA-MB-468 4.5 | 770.93 | 681.01 | 697.83 |
| MDA-MB-468 4.7 | 838.16 | 752.74 | 784.39 |
| MDA-MB-468 4.8 | 860.76 | 769.51 | 813.12 |
| MDA-MB-468 4.10 | 1016.21 | 904.69 | 947.46 |
| MDA-MB-468 4.11 | 870.10 | 776.73 | 813.12 |
| MDA-MB-468 4.12 | 986.93 | 857.20 | 913.98 |
| MDA-MB-468 4.13 | 790.41 | 712.25 | 743.18 |
| MDA-MB-468 4.14 | 942.36 | 842.34 | 873.79 |
| MDA-MB-468 4.16 | 771.81 | 677.69 | 697.83 |

“MDA-MB-468 control.1” is MDA-MB-468 cells without staining—neither primary nor secondary antibody. “MDA-MB-468 control.2” is MDA-MB-468 cells stained with irrelevant primary antibody MART-1 and the Alexa Fluor 488 secondary antibody. All other cells, as described, were stained with Anti-erbB2 primary antibody and Alexa Fluor 488 secondary antibody.

These data showed that MDA-MB-468 cells transformed with pCMV.BER2.BGI2.2REH have significantly reduced expression of HER-2 protein.

4. Anasis by Nuclear Transcription Run-On Assays

To detect transcription of the transgene RNA in the nucleus of MDA-MBE468 cells nuclear transcription run-on assays are performed on cell-free nuclei isolated from actively dividing cells. The nuclei are obtained according to the cell nuclei isolation protocol set forth in Example 10, above.

Analysis of nuclear RNA transcripts for the transgene HER2.BGI2.2REH and the endogenous HER-2 gene is performed according to the nuclear transcription run-on protocol set forth in Example 10, above.

5. Comparison of mRNA in Non-Transformed and Co-Suppressed Lines

Messenger RNA for the endogenous HER-2 gene and RNA transcribed from the transgene HER23GI2.2REH are analyzed according to the protocols set forth in Example 10, above.

6. Southern Analysis

Individual transgenic NIH/3T3 cell lines are analyzed by Southern blot analysis to confirm integration and determine copy number of the transgene. The procedure is carried out according to the protocol set forth in Example 10, above.

7. Western Blot Analysis

Selected clones and control MDA-MB-468 cells are grown overnight to near-confluence on 100 mm TC plates (10⁷ cells). Cells in plates are first washed with buffer containing phosphatase inhibitors (50 mM Tris-HCl pH 6.8, 1 mM Na₄P₂O₇, 10 mM NaF, 20 μM Na₂MoO₄, 1 mM Na₃VO₄), and then scraped from the plate in 600 μl of lysis buffer (50 mM Tris-HCl pH 6.8, 1 mM Na₄P₂O₇, 10 mM NaF, 20 μM Na₂MoO₄, 1 mM Na₃VO₄, 2% w/v SDS) which has been heated to 100° C. Suspensions are incubated in screw-capped tubes at 100° C. for 15 min. Tubes with lysed cells are centrifuged at 13,000 rpm for 10 min; supernatant extracts are removed and stored at −20° C.

SDS-PAGE 10% v/v separating and 5% v/v stacking gels (0.75 mm) are prepared in a Protean apparatus (BioRad) using 29:1 acrylamide:bisacrylamide (Bio-Rad) and Tris-HCl buffers at pH 8.8 and 6.8, respectively. Volumes of 60 μl from extracts are combined with 20 μl of 4×loading buffer (50 mM Tris-HCl pH 6.8, 2% w/v SDS, 40% v/v glycerol, bromophenol blue and 400 mM dithiothreitol added before use), heated to 100° C. for 5 min, cooled then loaded into wells before the gel is run in the cold room at 120V until protein samples enter the separating gel, then at 240V. Separated proteins are transferred to Hybond-ECL nitrocellulose membranes (Amersham) using an electroblotter (Bio-Rad), according to manufacturer's instructions.

Membranes are rinsed in TBST buffer (10 mM Tris-HCl pH 8.0, 150 mM NaCl, 0.05% v/v Tween 20) then blocked in a dish in TBST with 5% w/v skim milk powder plus phosphatase inhibitors (1 mM Na₄P₂O₇, 10 mM NaF, 20 μM Na₂MoO₄, 1 mM Na₃VO₄). Membranes are incubated in a small volume in TBST with 2.5% w/v skim milk powder plus phosphatase inhibitors containing a mouse monoclonal antibody against the ECD of HER-2 (Transduction Laboratories, NeoMarkers) diluted 1:4000. Membranes are washed three times for 10 min in TBST with 2.5% w/v skim milk powder plus phosphatase inhibitors. Membranes are incubated in a small volume in TBST with 2.5% w/v skim milk powder plus phosphatase inhibitors containing the horse radish peroxidase conjugated secondary antibody diluted 1:1000. Membranes are washed three times for 10 min in TBST with 2.5% w/v skim milk powder plus phosphatase inhibitors.

The presence of HER-2 protein is detected using the ECL luminol-based system (Amersham), according to manufacturer's instructions. Stripping of membranes for detection of a second control protein is done by incubating membranes for 30 min at 55° C. in 100 ml of stripping buffer (62 mM Tris-HCl pH 6.7, 2% w/v SDS, 100 mM freshly prepared 2-mercaptoethanol).

### EXAMPLE 19

Co-Suppression of Brn-2 in MM96L Melanoma Cells In Vitro

The Brn-2 transcription factor-belongs to a class of DNA binding proteins, termed Oct-factors, which specifically interact with the octamer control sequence ATGCAAAT. All Oct-factors belong to a family of proteins that was originally classified on the basis of a conserved region essential for sequence-specific, high affinity DNA binding termed the POU domain. The POU domain is present in three mammalian transcription factors, Pit-1, Oct-1 and Oct-2 and in a developmental control gene in C. elegans, unc-86. Additional POU proteins have been described in a number of species and these are expressed in a cell-lineage specific manner. The brn-2 gene appears to be involved in the development of neuronal pathways in the embryo and the Brn-2 protein is present in the adult brain. Electromobility shift assays (EMSAs) of nuclear extracts from cultured mouse neurons and from tumours of neural crest origin have detected a number of Oct-factor proteins. These include N-Oct-2, N-Oct-3, N-Oct-4 and N-Oct-5. It has been shown that N-Oct-2, N-Oct-3 and N-Oct-5 are also differentially expressed in human melanocytes, melanoma tissue and melanoma cell lines, all derived from the neural crest lineage. The brn-2 genomic locus is known to encode the N-Oct-3 and N-Oct-5 DNA binding activities. N-Oct-3 is present in all melanoma cells tested so far including the MM96L line employed in these experiments. When expression of Brn-2 protein is blocked, N-Oct-3 DNA-binding activity is lost, and there are additional downstream effects including changes in cell morphology, a loss of expression of elements of the melanogenesis/pigmentation pathway and losses of neural crest markers and other markers of the melanocyfic lineage. Melanoma cells without Brn-2 are no longer tumorigenic in immunodeficient mice (Thomson et al., 1995).

1. Culturing of Cell Lines

Cells of the MM96L line, derived from human melanoma, were grown as adherent monolayers in RPMI 1640 medium supplemented with 10% v/v FBS and 2 mM L-glutamine, as described in Example 10, above.

2. Preparation of Genetic Constructs

(a) Interim Plasmid

Plasmid TOPO.BRN-2

A region of the human Brn-2 gene was amplified by PCR, using a human Brn-2 genomic clone, using the primers:—

brn1: AGA TCT GAC AGA AAGAGC GAG CGA GGA GAG [SEQ ID NO: 17]

and

brn4: GGA TTC AGT GCG GGT CGT GGT GCG CGC CTG [SEQ ID NO: 18].

The amplification product was cloned into pCR (registered trademark) 2.1-TOPO to create the intermediate clone TOPO.BRN-2.

(b) Test Plasmid

Plasmid pCMVBRN2.BGI2.2NRB

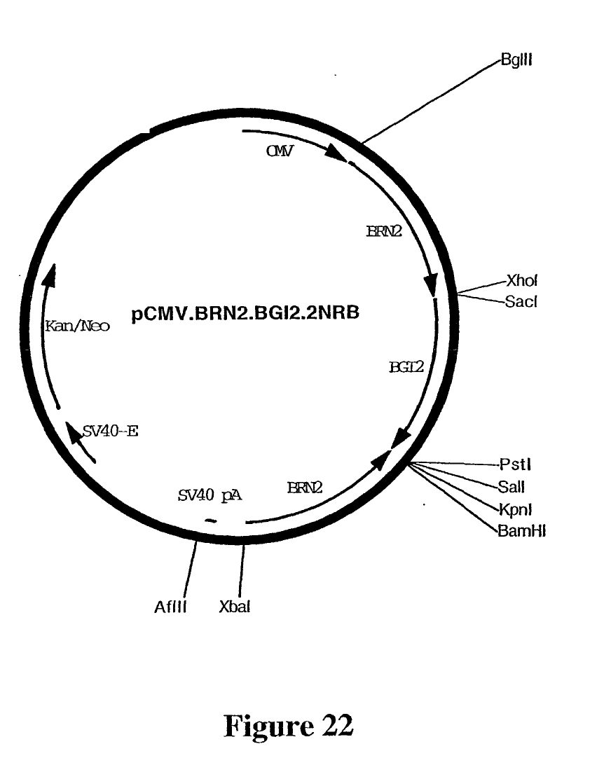

Plasmid pCMV.BRN2.BGI2.2NRB (FIG. 22) contains an inverted repeat or palindrome of the BRN-2 coding region that is interrupted by the insertion of the human β-globin intron 2 sequence therein. Plasmid pCMV.BRN2.BGI2.2NRB was constructed in successive steps: (i) the BRN2 sequence from plasmid TOPO.RN2 was sub-cloned in the sense orientation as a BglII-to-BamHI fragment into BglII-digested pCMV.BGI2.cass (Example 11) to make plasmid pCMV.BRN2.BGI2), and (ii) the BRN2 sequence from plasmid TOPO.BRN2 was sub-cloned in the antisense orientation as a BglII-to-BamHI fragment into BamHI-digested pCMV.BRN2.BGI2 to make plasmid pCMV.BRN2.BGI2.2NRB.

3. Detection of Co-Suppression Phenotype

(a) Transfection of Brn-2 constructs: Insertion of Brn2-Expressing Transgene Into MM96L Cells

Transformations were performed in 6-well tissue culture vessels. Individual wells were seeded with 1×10⁵ MM96L cells in 2 ml of RPMI 1640 medium, 10% v/v FBS and incubated at 37° C., 5% v/v CO₂ until the monolayer was 60-90% confluent, typically 16 to 24hr.

Subsequent procedures were as described above in Example 13, 3(a), except that MM96L cells were incubated with the DNA liposome complexes at 37° C. and 5% v/v CO₂ for 3 to 4 hr, only.

A total of 36 lines transformed with the construct pCMV.BRN2BGI2.2NRB were chosen for subsequent analyses.

(b) Post-Transcriptional Silencing of Brn-2-Expressing Transgene in MM96L Cells

Clones with features of PTGS of Brn-2 derived from MM96L cells stably transfected with the construct were selected on the basis of morphological changes from the phase bright, bipolar and multidendritic cell type common to melanocytes to a low contrast (LC), rounded shape which is distinct and easily identified. Cells arising from such LC clones are subjected to analysis by electromobility shift assay (EMSA, see below) to identify presence or absence of N-Oct-3 activity. Additional testing is based on the loss of pigmentation. Cells of LC clones are stained for the presence of melanin using the modified Schmorl's method for staining of the pigment biopolymer, as described in Example 14, above. Clones that fulfil all criteria—(i) LC morphology, (ii) absence of N-Oct-3 DNA binding activity, and (iii) loss of pigmentation—undergo direct testing of PTGS via nuclear transcription run-on assays.

To isolate lines for further analyses, lines showing altered morphology were selected and sub-clones of these lines were obtained by plating the parental clones at low density and picking clones showing altered morphology using techniques outlined above (see Example 10). The sub-clones chosen for further analyses were MM96L 2.1.1 and MM96L 3.19.1.

4. Analysis by Nuclear Transcription Run-On Assays

To estimate transcription rates of the endogenous BRN-2 gene in MM96L cells and the transformed lines MM96L 2.1.1 and MM96L 3.19.1, nuclear transcription run-on assays are performed on nuclei isolated from actively dividing cells. The nuclei are obtained according to the cell nuclei isolation protocol set forth in Example 10, above, and transcription nm-on transcripts are labelled with biotin and purified using streptavidin capture as outlined in Example 10.

To determine the transcription rate of the endogenous BRN-2 gene in the above cell lines, the amount of biotin-labelled BRN-2 transcript isolated from nuclear run-on assays is quantified using real time PCR reactions. The relative transcription rates of the endogenous BRN-2 gene is estimated by comparing the level of biotin-labelled BRN-2 RNA to the level of a ubiquitously-expressed endogenous transcript, namely human glyceraldehyde phosphate dehydrogenase (GAPDH).

The levels of expression of both the endogenous BRN-2 and human GAPDH genes are determined in duplex PCR reactions.

5. Comparison of mRNA in Non-Transformed and Co-Suppressed Lines

Messenger RNA for the endogenous Brn-2 gene and RNA transcribed from the transgene BRN2.BGI2.2NRB are analyzed according to the protocols set forth in Example 10, above.

To obtain accurate estimates of BRN-2 mRNA levels in Mb96L and transformed lines, real time PCR reactions were employed. Results from these analyses are shown in Table 11.

TABLE 11

| | BRN-2 and GAPDH mRNA levels | | | |
|---|---|---|---|---|
| | in olig-dT purified total RNAs | | | Relative levels |
| Cell Line | Cₜ TYR | Cₜ GAPDH | Δ Cₜ | of BRN-2 mRNA |
| MM96L | 33.1 | 22.7 | 10.4 | 1.00 |
| MM96L 2.1.1 | 33.2 | 22.5 | 10.7 | 0.83 |
| MM96L 3.19.1 | 32.1 | 22.6 | 9.5 | 0.89 |

These data show that the levels of BRN-2 mRNA (as poly(A)RNA) in two transformed lines with reversion phenotype, MM96L 2.1.1 and MM96L 3.19.1, are not significantly different from the level of BRN-2 mRNA in non-transformed MM96L cells.

6. Southern Analysis

Individual transgenic MM96L cell lines are analyzed by Southern blot analysis to confirm integration and determine copy number of the transgene. The procedure is carried out according to the protocol set forth in Example 10, above.

7. Electromobility Shift Assay (EMSA)

To prepare nuclear and cytoplasmic extracts, 2×10⁷ cells are plated in a 100 mm TC dish and grown overnight. Before harvesting cells, the TC dish is put on ice, the medium aspirated completely and cells washed twice with ice cold PBS. A volume of 700 μl PBS is added and cells scraped off the plate and the suspension transferred to a 1.5 ml microfuge tube. The plate is rinsed with 400 μl ice cold PBS and this is added to the tube. All subsequent work is done at 4° C. The cell suspension is centrifuged at 2,500 rpm for 5 min and the supernatant removed. A volume of 150 μl HWB solution [10 mM YEPES pH 7.4, 1.5 mM MgCl₂, 10 mM KCl, protease inhibitors (Roche), 1 mM sodium orthovanadate and phosphatase inhibitors comprising 10 mM NaF, 15 mM Na₂MoO₄ and 100 μM Na₃VO₄] is added to the pellet and cells resuspended with a pipette. Cell swelling is checked at this point. A volume of 300 μl LB solution [10 mM HEPES pH 7.4, 1.5 mM MgCl₂, 10 mM KCl, protease inhibitors (Roche), 1 mM sodium orthovanadate and phosphatase inhibitors and 0.1% NP-40] is added and cells left on ice for 5 min. Cell lysis is checked at this point The tube is spun at 2500 rpm for 5 min and the supernatant transferred to a new tube. The pellet, which comprises the cell nuclei, is retained.

Nuclei are washed by resuspension in 800 μl of HWB solution, then the tube is spun at 2,500 rpm for 5 min. The supernatant is removed and the nuclei are resuspended in 150 μl NEB solution [20 mM HEPES pH 7.8, 0.42 M NaCl, 20% v/v glycerol, 0.2 mM EDTA, 1.5 mM MgCl₂, protease inhibitors, 1 mM sodium orthovanadate and phosphatase inhibitors] and left on ice for 10 min. The tube is spun at 13,000 rpm to pellet nuclear remnants, then the supernatant, which is the nuclear extract, is removed. A small aliquot of each nuclear extract is retained for determination of protein concentration by the colorimetric Bradford assay (Bio-Rad). The remainder is stored at −70° C. NEB solution is stored and used to dilute extracts for working concentrations.

The double-stranded DNA probes used for EMSA of N-Oct-1 and N-Oct-3 were as follows:—

| | | |
|---|---|---|
| clone 25 | GCATAATTAATGAATTAGTG | [SEQ ID NO:19] |
| | CGTATTAATTACTTAATCAC | |
| Oct-WT | GAAGTATGCAAAGCATGCATCTC | [SEQ ID NO:20] |
| | CTTCATACGTTTCGTACGTAGAG | |
| Oct-dpm8 | GAAGTAAGGAAAGCATGCATCTC | [SEQ ID NO:21] |
| | CTTCATTCCTTTCGTACGTAGAG | |

The clone 25 probe has a high affinity for Oct-1 and N-Oct-3. The sequence was selected for these properties from a panel of randomly-generated double stranded oligonucleotides (Bendall et al., 1993). The probe Oct-WT was derived from the SV40 enhancer sequence and contains a consensus octamer binding site which has been mutated in the Oct-dpmg probe (Sturm et al., 1987; Thomson et al., 1995).

Probes are labelled with [γ-³²P]-ATP. The probes are diluted to 1 μM and 5 μl is incubated at 37° C. for 1 hr in 1×polynucleotide kinase (PNK) buffer (Roche), 2 μl [γ-³²P]-ATP (10 mCi/ml, 3000 Ci/mmol, Amersham) with 1 μl T4 PNK (10 U/μl (Roche)) brought to a volume of 20 μl with MilliQ water. The reaction is diluted to 100 μl with TE buffer (see Example 10) and run through a Sephadex G25 column (Nap column (Roche)) with TE. Approximately 4.5 pmol of labelled probe is recovered at a concentration of 0.15 pmol/μl. Labelled probes are stored at −20° C.

Binding reactions of probe and extracts are done in 10 μl volumes comprising 12% v/v glycerol, 1×binding buffer (20 mM BEPES pH 7.0, 140 mM KCl), 13 mM NaCl, 5 mM MgCl₂, 2 μl labelled probe (0.04 pmol), 1 μg protein extract, MilliQ water and, where indicated, unlabelled probe competitor. The order of addition is usually competitor or water, labelled probe, protein extract. One tube is prepared without a protein sample but with 2 μl PAGE loading dye (see Example 10).

Binding reactions are incubated for 30 min at room temperature before 9 μl is loaded into the wells of a Mini-Protean (Bio-Rad) apparatus prepared with a 7% acrylamide: bisacrylamide 29:1 Tris-glycine gel. The 1×gel and 1×gel running buffer are diluted from 5×stocks, respectively, 0.75 M Tris-HCl pH 8.8 and 125 mM Tris-HCl pH 8.3, 0.96 M glycine, 1 mM EDTA pH 8. Gels are run at 10 V/cm, fixed in 10% v/v acetic acid for 15 min, transferred to Whatman 3MM paper and dried before exposure of X-ray film for 16-48 hr.

### EXAMPLE 20

Co-Suppression of YB-1 and p53 in Murine Type B10.2 and Pam 212 Cells In Vitro

1. Culturing of Cell Lines

B10.2 cells derived from murine fibrosarcoma and Pam 212 cells derived from murine epidermal keratinocytes were grown as adherent monolayers using either RPMI 1640 or DMEM supplemented with 5% v/v FBS, as described in Example 10, above.

2. Preparation of Genetic Constructs

(a) Interim Plasmids

Plasmid TOPO.YB-1

To amplify a region of the mouse YB-1 gene, 25 ng of a plasmid clone containing a mouse YB-1 cDNA (obtained from Genesis Research & Development Corporation, Auckland NZ) was used as a substrate for PCR amplification using the primers:—

Y1: AGA TCT GCA GCA GAC CGT AAC CAT TAT AGG [SEQ ID NO: 22]

and

Y4: GGA TCC ACC TTT ATT AAC AGG TGC TTG CAG [SEQ ID NO: 23].

The PCR amplification was performed using HotStarTaq DNA polymerase according to the manufacturer's protocol (Qiagen). PCR amplification conditions involved an initial activation step at 95° C. for 15 mins, followed by 35 amplification cycles of 94° C. for 30 secs, 55° C. for 30 secs and 72° C. for 60 secs, with a final elongation step at 72° C. for 4 mins.

The PCR amplified region of YB-1 was column purified (PCR purification column, Qiagen) and then cloned into pCR (registered trademark) 2.1-TOPO according to the manufacturer's instructions (Invitrogen), to make plasmid TOPO.YB-1.

Plasmid TOPO.p53

To amplify a region of the mouse p53 gene, 25 ng of a plasmid clone containing a mouse p53 cDNA (obtained from Genesis Research & Development Corporation, Auckland NZ) was used as a substrate for PCR amplification using the primers:—

P2: AGA TCT AGA TAT CCT GCC ATC ACC TCA CTG [SEQ ID NO: 24]

and

P4: GGA TCC CAG GCC CCA CTT TCT TGA CCA TTG [SEQ ID NO: 25].

The PCR amplification was performed using HotStarTaq DNA polymerase according to the manufacturer's protocol (Qiagen). PCR amplification conditions involved an initial activation step at 95° C. for 15 mins, followed by 35 amplification cycles of 94° C. for 30 secs, 55° C. for 30 secs and 72° C. for 60 secs, with a final elongation step at 72° C. for 4 mins.

The PCR amplified region of p53 was column purified (PCR purification column, Qiagen) and then cloned into pCR (registered trademark) 2.1-TOPO according to the manufacturer's instructions (Invitrogen), to make plasmid TOPO.p53.

Plasmid TOPO.YB1.p53

To create a construct fusing YB-1 and p53 cDNA sequences, the murine YB-1 sequence from TOPO.YB-1 was isolated as a BglII-to-BamHI fragment and cloned into the BamHI site of TOPO.p53. A clone in which the YB-1 insert was oriented in the same sense as the p⁵³ sequence was selected and designated TOPO.YB1.p53.

(b) Test Plasmids

Plasmid pCMV.YB1.BGI2.1BY

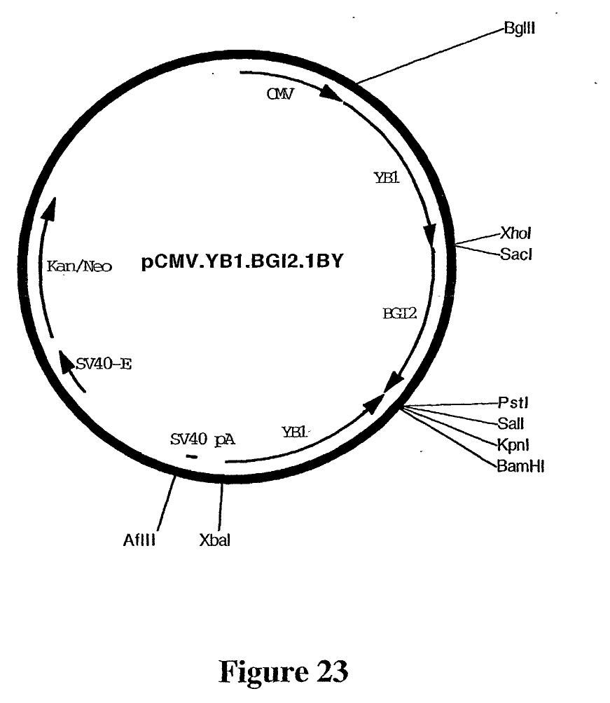

Plasmid pCMV.YB1.BGI2.IBY (FIG. 23) is capable of transcribing a region of the murine YB-1 gene as an inverted repeat or palindrome that is interrupted by the human β-globin intron 2 sequence therein. Plasmid pCMV.YB1.BGI2.1BY was constructed in successive steps: (i) the YB-1 sequence from plasmid TOPO.YB-1 was sub-cloned in the sense orientation as a BglII-to-BamHI fragment into BglII-digested pCMV.BGI2 to make plasmid pCMV.YB1.BGI2, and (ii) the YB-1 sequence from plasmid TOPO.YB-1 was sub-cloned in the antisense orientation as a BglII-to-BamHI fragment into BamHI-digested pCMV.YB1.BGI2 to nake plasmid pCMV.YB1.BGI2.1BY.

Plasmid pCMV.YB1.p53.BGI2.35p.1BY

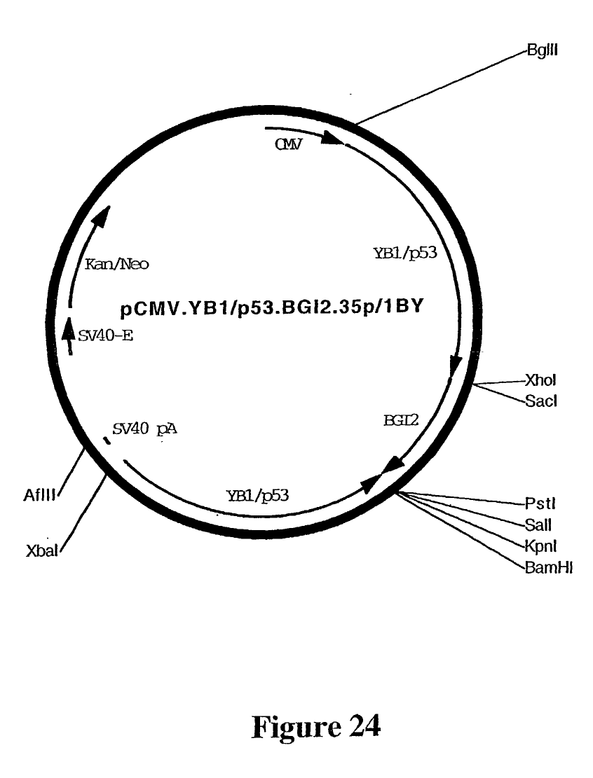

Plasmid pCMV.YB1.p53.BGI2.35p.1BY (FIG. 24) is capable of expressing fused regions of the murine YB-1 and p53 genes as an inverted repeat or palindrome that is interrupted by the human β-globin intron 2 sequence therein. Plasmid pCMV.YB1.p53.BG12.35p.1BY was constructed in successive steps: (i) the YB-1.p53 fusion sequence from plasmid TOPO.YB1.p53 was sub-cloned in the sense orientation as a BglII-to-BamHI fragment into BglII-digested pCMV.BGI2 to make plasmid pCMV.YB1.p53.BGI2, and (ii) the YB-1.p53 fusion sequence from plasmid TOPO.YB1.p53 was sub-cloned in the antisense orientation as a BglII-to-BamHI fragment into BamHI-digested pCMV.YB1.p53.BGI2 to make plasmid pCMV.YB1.p53.BGI2.35p.1BY.

3. Detection of Co-Suppression Phenotypes

(a) Post-Transcriptional Gene silencing of YB-1 by Insertion of a Region of the YB-1 Gene into Murine Fibrosarcoma B10.2 Cells and Murine Epidermal Keratinocyte Pam 212 Cells

YB-1 (Y-box DNA/RNA-binding factor 1) is a transcription factor that binds, inter alia, to the promoter region of the p53 gene and in so doing represses its expression. In cancer cells that express normal p53 protein at normal levels (some 50% of all human cancers), the expression of p53 is under the control of YB-1, such that diminution of YB-1 expression results in increased levels of p53 protein and consequent apoptosis. The murine cell lines B10.2 and Pam 212 are two such tumorigenic cell lines with, normal p53 expression. The expected phenotype for co-suppression of YB-1 in these two cell lines is apoptosis.

Transformations with pCMV.YB1.BGI2.1BY were performed in 6 well tissue culture vessels. Individual wells were seeded with 3.5×10⁴ cells (B10.2 or Pam 212) in 2 ml of RPMI 1640 or DMEM, 5% v/v FBS and incubated at 37° C., 5% v/v CO₂ for 24 hr prior to transfection.

The two mixes used to prepare transfection medium were:

Mix A: 1.5 μl of LIPOFECTAMINE 2000 (trademark) Reagent (Life Technologies) in 100 μl of OPTI-EM I (registered trademark) medium (Life Technologies), incubated at room temperature for 5 min;

Mix B: 1 μl (400 ng) of pCMV.YB1.BG12.1BY DNA in 100 μl of OPTI-MEM I (registered trademark) medium.

After preliminary incubation, Mix A was added to Mix B and the mixture incubated at room temperature for a further 20 min.

Medium overlaying each cell culture was replaced with 800 μl of fresh medium and 200 μl of transfection mix added. Cells were incubated at 37° C., 5% v/v CO₂ for 72 hr.

Duplicate cultures of both cell types (B10.2 and Pam 212) were transfected.

Cells were suspended with trypsin, centrifuged and resuspended in PBS according to the protocol described in Example 10.

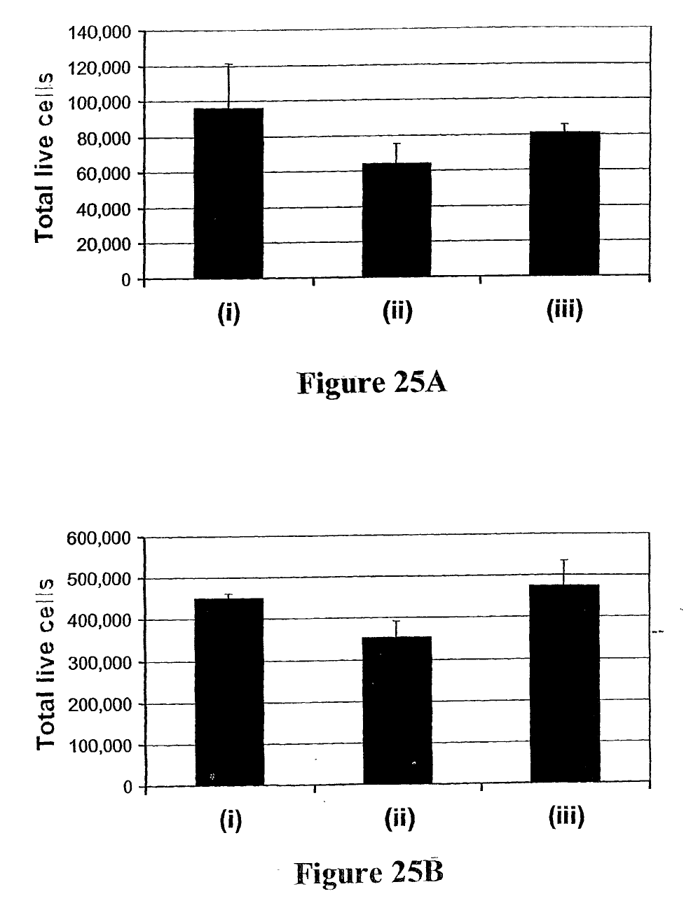

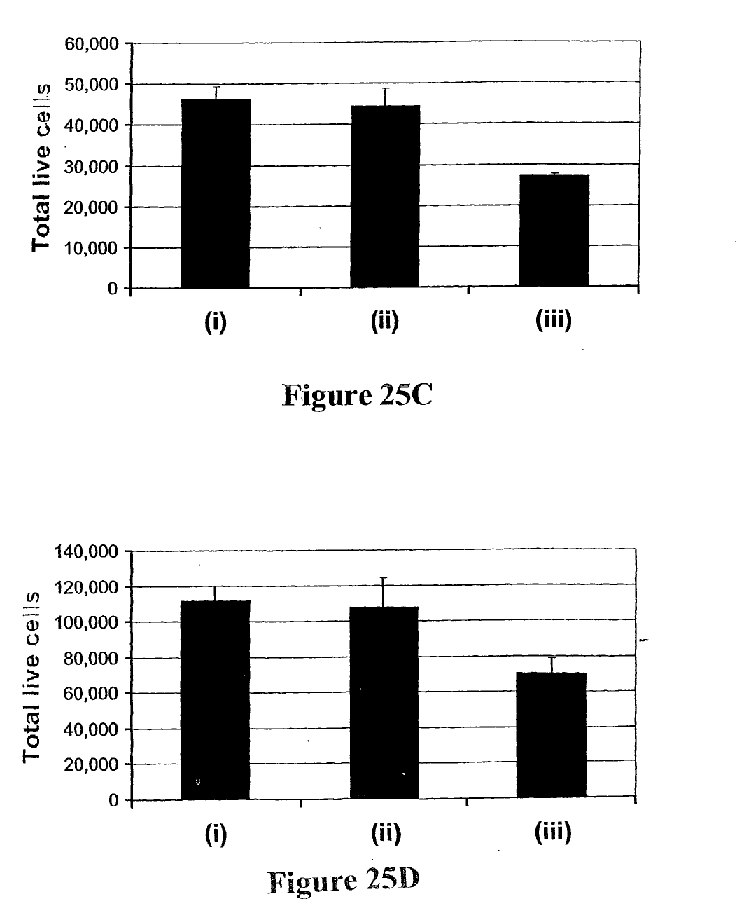

Live and dead cell numbers were determined by trypan blue staining (0.2%) and counting in quadruplicate on a haemocytometer slide. Results are presented in FIGS. 25A, 252B, 25C and 25D (refer to the Figure Legends for details).

(b) Post-Transcriptional Gene Silencing of YB-1 and p53 by Co-Insertion of Regions of the YB-1 and p53 Genes into Murine Ribrosarcoma B10.2 Cells and Murine Epidermal Keratinocyte Pam 212 Cells

The data presented in FIGS. 25A, 25B, 25C and 25D show that cell death is increased in B10.2 and Pam 212 cells following insertion of a YB-1 construct designed to induce co-suppression of YB-1, consistent with induction of co-suppression. Simultaneous co-suppression of p53, which is responsible for initiating the apoptotic response in these cells, would be expected to eliminate excess cell death by apoptosis.

Transformations with pCMV.YB1.p53.BGI2.35p.1BY were performed in 6 well tissue culture vessels. Individual wells were seeded with 3.5×10⁴ cells (B 10.2 or Pam 212) in 2 ml of RPMI 1640 or DMEM, 5% v/v FBS and incubated at 37° C., 5% v/v CO₂ for 24 hr prior to transfection.

The two mixes used to prepare transfection medium were:—

Mix A: 1.5 μl of LIPOFECTAMINE 2000 (trademark) Reagent in 100 μl of OPTI-MEM I (registered trademark) medium, incubated at room temperature for 5 min;

Mix B: 1 μl (400 ng) of pCMV.YB1.p53.BGI2.35p.1BY DNA in 100 μl of OPTI-MEM I (registered trademark) medium.

After preliminary incubation, Mix A was added to Mix B and the mixture incubated at room temperature for a further 20 min

Medium overlaying each cell culture was replaced with 800 μl of fresh medium and 200 μl of transfection mix added. Cells were incubated at 37° C., 5% v/v CO₂ for 72 hr.

Cells were suspended with trypsin, centrifuged and resuspended in PBS according to the protocol described in Example 10.

Live and dead cell numbers were determined by trypan blue staining (0.2%) and counting in quadruplicate on a haemocytometer slide. Results are presented in FIGS. 25A, 252B, 25C and 25D (refer to the Figure Legends for details).

(c) Control: Insertion of GFP into Murine Fibrosarcoma B10.2 Cells and Murine Epidermal Keratinocyte Pam 212 Cells

Transformations with pCMV.EGFP were performed in 6 well tissue culture vessels. Individual wells were seeded with 3.5×10⁴ cells (B10.2 or Pam 212) in 2 ml of RPMI 1640 or DMEM, 5% v/v FBS and incubated at 37° C., 5% v/v CO₂ for 24 hr prior to transfection.

The two mixes used to prepare transfection medium were:—

Mix A: 1.5 μl of LIPOFECTAMINE 2000 (trademark) Reagent in 100 μl of OPTI-MEM I (registered trademark) medium, incubated at room temperature for 5 min;

Mix B: 1 μl (400 ng) of pCMV.EGFP DNA in 100 μl of OPTI-MEM I (registered trademark) medium.

After preliminary incubation, Mix A was added to Mix B and the mixture incubated at room temperature for a further 20 min.

Medium overlaying each cell culture was replaced with 800 μl of fresh medium and 200 μl of transfection mix added. Cells were incubated at 37° C., 5% v/v CO₂ for 72 hr.

Cells were suspended with trypsin, centrifuged and resuspended in PBS according to the protocol described in Example 10.

Live and dead cell numbers were determined by trypan blue staining (0.2%) and counting in quadruplicate on a haemocytometer slide. Results are presented in FIGS. 25A, 252B, 25C and 25D (refer to the Figure Legends for details).

(d) Control: Attenuation of YB-1 Phenotype by Insertion of a Decoy Y-Box Oligonucleotide into Murine Fibrosarcoma B10.2 Cells and Murine Epidermal Keratinocyte Pam 212 Cells

The role of YB-1in repressing p53-initiated apoptosis in B110.2 and Pam 212 cells has been demonstrated by relieving the repression in two ways: (i) transfection with YB-1 antisense oligonucleotides; (ii) transfection with a decoy oligonucleotide that corresponds to the Y-box sequence of the p53 promoter. The latter was used as a positive control in the present example.

Transformations with YB1 decoy and a control (non-specific) oligonucleotide were performed in 24 well tissue culture vessels. Individual wells were seeded with 3.5×10⁴ cells (B10.2 or Pam 212) in 2 ml of RPMI 1640 or DMEM, 5% v/v FBS and incubated at 37° C., 5% v/v CO₂ for 24 hr prior to transfcction.

The two mixes used to prepare transfection medium were:—

Mix A: 1.5 μl of Lipofectin (trademark) Reagent (Life Technologies) in 100 μl of OPTI-MEM I (registered trademark) medium, incubated at room temperature for 30 min;

Mix B: 0.4 μl (40 pmol) of oligonucleotide (YB1 decoy or control) in 100 μl of OPTI-MEM I (registered trademark)medium.

After preliminary incubation, Mix A was added to Mix B and the mixture incubated at room temperature for a further 15 min.

A no-oligonucleotide (Lipofectin (trademark) only) control was also prepared.

Cells were washed in serum-free medium (Optimem) and transfection mix added. Cells were incubated at 37° C., 5% v/v CO₂ for 4 hr, after which medium was replaced with 1 ml of RPMI containing 10% v/v FBS and incubation continued overnight (18 hr).

Cells were suspended with trypsin, centrifuged and resuspended in PBS according to the protocol described in Example 10.

Live and dead cell numbers were determined by trypan blue staining (0.2%) and counting in quadruplicate on a haemocytometer slide. Results are presented in FIGS. 25A, 252B, 25C and 25D (refer to the Figure Legends for details).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altschul, S. F., Madden, T. L, Schaffer, A. A., Zhang, J. H., Zhang, Z., Miller, W. and Lipman, D. J. (1997) Nucl. Acids Res. 25:3389.

Ausubel, F. M., Brent, R, Kingston, R, E., Moor, D. D., Seidman, J. G., Smith, J. A., Struhtl, K. (eds) “Current Protocols in Molecular Biology” John Wiley & Sons Inc, 1994-1998, Chapter 15.

Bahramian, M. B. and Zarbl, H. (1999) Mol. Cell. Biol. 19:274-283.

Bingham, P. M. (1997) Cell 90:385-387.

Bonner and Laskey, (1974) Eur. J Biochem. 46:83.

Bendall, A. J., Sturn, R. A., Danoy, P. A. C. and Molloy, P. L. (1993) Broad binding-site specificity and affinity properties of octamer 1 and brain octamer-binding proteins. European Journal of Biochemistry 217:799-811.

Bennett, D. C., Cooper, P. J., Dexter, T. J., Devlin, L. M., Heasman, J., and Nester, B. (1989) Development 105:379-385.

Cogoni, C., Irelan, J. T., Schumacher, M., Schmidhauser, T. J., Selker, E. J. and Macino, G. (1996) The EMBO Journal 15:3153-3163.

Cogoni, C. and Macino, G. (1997) Proceedings of the National Academy of Sciences 94:10233-10238.

Cormack, B. P., Valdivia, R. H. and Falkow, S. (1996) Gene 173 (1 Spec. No.):33-38.

de la Luna, S. and Ortin, J. (1992) Methods in Enzymology 216:376-385.

Doherty, J. K., Bond, C., Jardim, A., Adelman, J. P. and Clinton, G. M. (1999) Proceedings of the National Academies of Science of the USA 96(19):10869-10874.

Dutkiewicz, R., Albert, D. M. and Levin, L. A. (2000) Effects of latanoprost on tyrosine activity and mitotic index of cultured melanoma lines. Experimental Eye Research 70: 563-569.

Garrick, D., Fiering, S., Martin, D. I. K and Whitelaw, E. (1998) Nature Genetics 18:56-59.

Koss, L. G. (1979) Diagnostic Cytology. J. B. Lippincott, Philadelphia.

Lindbo, J. A., Silva-Rosales, L. Proebsting, W. M. and Dougherty, W. G. (1993) The Plant Cell 5:1749-1759.

Littlefield, J. W. (1964) Science 154:709-710.

Marmur and Doty (1962) J. Mol. Biol. 5:109.

Napoli, C., Lemieux, C. and Jorgensen, R. (1990) The Plant Cell 2:279-289.

Pal-Bhadra, M., Bhadra, U. and Birchler, J. A. (1997) Cell 90:479-490.

Patrone, G., Puppo, F., Cusano, R., Scamari, M., Ceccherini, I., Puliti, A and Ravazzolo, R. (2000) BioTechiques 29:1012-1017.

Sambrook, J., Fritsch, E. F. and Maniatis, T. (1989). Molecular Cloning—A laboratory Manual, 2nd ed. Cold Spring Harbor Laboratory Press, Cold Spring Harbor.

Slamon, D. J., Clark, G. M., Wong, S. G., Levin, W. J., Uhkich, A. and McGuire, W. L. (1987) Science 235:77-182.

Spanakis, E., Lamina, P., and Bennett, D. C. (1992) Development 114:675-680.

Stunn, R., Baurnker, T., Franza Jr, B. R, Herr, W. (1987) Genes and Development 1:1147-1160.

Sviderskaya, E. V., Wakeling, W. F., and Benmett, D. C. (1995) Development 114:1547-1557.

Tearle, R. G., Tange, M. J., Zannettino, Z. L., Katerelos, M., Shinkel, T. A., van Denderen, B. J. W., Lonie, A. J., Lyons, I., Nottle, M. B, Cox, T., Becker, C., Peura, A. M, Wigley, P. L., Crawford, R. J., and d'Apice, A. J. F. (1996) Transplantation 61:13-19.

Thomson, J. A. F., Murphy, K, Baker, E., Sutherland, Grant R., Parsons, Peter G. and Stuin, Richard A. (1995) Oncogene 11:691-700.

van der Krol, A. R., Mur, L A., Beld, M., Mol, J. N. and Stuitje, A. R. (1990) The Plant Cell 2:291-299.

Waterhouse, P. M., Graham, M. W. and Wang, Ming-Bo (1998) Proceedings of the National Academy of Sciences 95:13959-13964.

Winder, A. J. and Harris, H. (1991) New assays for the tyrosine hydroxylase and dopa oxidase activities of tyrosinase. European Journal of Biochemistry 198: 317-326.

All publications, patents, and patent applications are incorporated by reference herein, as though individually incorporated by reference.

What is claimed is:

1. A genetic construct comprising a sequence of nucleotides substantially identical to a target endogenous sequence of nucleotides in the genome of a vertebrate animal cell and a nucleotide sequence complementary to said target endogenous nucleotide sequence wherein the nucleotide sequences identical and complementary to said target endogenous nucleotide sequences are separated by a spacer sequence wherein upon introduction of said genetic construct to said animal cell, an RNA transcript resulting from transcription of a gene comprising said endogenous target sequence of nucleotides exhibits an altered capacity for translation into a proteinaceous product.

2. The genetic construction of claim 1 wherein the vertebrate animal cell is from a mammal, avian species, fish or reptile.

3. The genetic construct of claim 2 wherein the vertebrate animal cell is from a mammal.

4. The genetic construct of claim 3 wherein the mammal is a human, primate, livestock animal or laboratory test animal.

5. The genetic construct of claim 4 wherein the mammal is a murine species.

6. The genetic construct of claim 4 wherein the mammal is a human.

7. The genetic construct of claim 1 wherein the spacer sequence is an intron.

8. The genetic construct of claim 7 wherein the intron sequence is an intron from a gene encoding β-globin.

9. The genetic construct of claim 8 wherein the β-globin intron is human β-globin intron 2.

10. The genetic construct of claim 1 wherein there is substantially no reduction in the level of transcription of said gene comprising the endogenous target sequence.

11. The genetic construct of claim 1 wherein the total level of RNA transcribed from said gene comprising said endogenous target sequence of nucleotides is not substantially reduced.

12. A genetic construct comprising:
(i) a nucleotide sequence substantially identical to a target endogenous sequence of nucleotides in the genome of a vertebrate animal cell;
(ii) a single nucleotide sequence substantially complementary to said target endogenous nucleotide sequence defined in (i);
(iii) an intron nucleotide sequence separating said nucleotide sequence of (i) and (ii);
wherein upon introduction of said construct to said animal cell, an RNA transcript resulting from transcription of a gene comprising said endogenous target sequence of nucleotides exhibits an altered capacity for transcription.

13. The genetic construct of claim 12 wherein the vertebrate animal cell is from a mammal, avian species, fish or reptile.

14. The genetic construct of claim 13 wherein the vertebrate animal cell is from a mammal.

15. The genetic construct of claim 14 wherein the mammal is a human, primate, livestock animal or laboratory test animal.

16. The genetic construct of claim 15 wherein the mammal is a murine species.

17. The genetic construct of claim 14 wherein the mammal is a human.

18. The genetic construct of claim 12 wherein there is substantially no reduction in the level of transcription of said gene comprising the endogenous target sequence.

19. The genetic construct of claim 12 wherein total level of RNA transcribed from said gene comprising said endogenous target sequence of nucleotides is not substantially reduced.

20. A genetic construct comprising:
(i) a nucleotide sequence substantially identical to a target endogenous sequence of nucleotides in the genome of a vertebrate animal cell;
(ii) a nucleotide sequence substantially complementary to said target endogenous nucleotide sequence defined in (i);
(iii) an intron nucleotide sequence separating said nucleotide sequence of (i) and (ii);
wherein upon introduction of said construct to said animal cell, an RNA transcript resulting from transcription of a gene comprising said endogenous target sequence of nucleotides exhibits an altered capacity for translation into a proteinaceous product and wherein there is substantially no reduction in the level of transcription of said gene comprising the endogenous target sequence and/or total level of RNA transcribed from said gene comprising said endogenous target sequence of nucleotides is not substantially reduced.

21. The genetic construct of claim 20 wherein the vertebrate animal cell is from a mammal, avian species, fish or reptile.

22. The genetic construct of claim 21 wherein the vertebrate animal cell is from a mammal.

23. The genetic construct of claim 22 wherein the mammal is a human, primate, livestock animal or laboratory test animal.

24. The genetic construct of claim 23 wherein the mammal is a murine species.

25. The genetic construct of claim 23 wherein the mammal is a human.

26. A genetically modified vertebrate animal cell characterized in that said cell:
(i) comprises a sense copy of a target endogenous nucleotide sequence introduced into said cell or a parent cell thereof; and
(ii) comprises substantially no proteinaceous product encoded by a gene comprising said endogenous target nucleotide sequence compared to a non-genetically modified form of same cell.

27. The genetically modified vertebrate animal cell of claim 26 wherein the vertebrate animal cell is from a mammal, avian species, fish or reptile.

28. The genetically modified vertebrate animal cell of claim 27 wherein the vertebrate animal cell is from a mammal.

29. The genetically modified vertebrate animal cell of claim 28 wherein the mammal is a human, primate, livestock animal or laboratory test animal.

30. The genetically modified vertebrate animal cell of claim 29 wherein the mammal is a murine species.

31. The genetically modified vertebrate animal cell of claim 29 wherein the mammal is a human.

32. The genetically modified vertebrate animal cell of claim 26 wherein the construct further comprises a nucleotide sequence complementary to said target endogenous nucleotide sequence.

33. The genetically modified vertebrate animal cell of claim 32 wherein the nucleotide sequences identical and complementary to said target endogenous nucleotide sequences are separated by an intron sequence.

34. The genetically modified vertebrate animal cell of claim 33 wherein the intron sequence is an intron from a gene encoding β-globin.

35. The genetically modified vertebrate animal cell of claim 34 wherein the β-globin intron is human β-globin intron 2.

36. The genetically modified vertebrate animal cell of claim 26 wherein there is substantially no reduction in the level of transcription of said gene comprising the endogenous target sequence.

37. The genetically modified vertebrate animal cell of claim 26 wherein total level of RNA transcribed from said gene comprising said endogenous target sequence of nucleotides is not substantially reduced.

38. A genetically modified vertebrate animal cell characterized in that said cell:
(i) comprises a sense copy of a target endogenous nucleotide sequence introduced into said cell or a parent cell thereof;
(ii) comprises substantially no proteinaceous product encoded by a gene comprising said endogenous target nucleotide sequence compared to a non-genetically modified form of same cell; and
(iii) comprises substantially no reduction in the levels of steady state total RNA relative to a non-genetically modified form of the same cell.

39. The genetically modified vertebrate animal cell of claim 38 wherein the vertebrate animal cell is from a mammal, avian species, fish or reptile.

40. The genetically modified vertebrate animal cell of claim 39 wherein the vertebrate animal cell is from a mammal.

41. The genetically modified vertebrate animal cell of claim 40 wherein the mammal is a human, primate, livestock animal or laboratory test animal.

42. The genetically modified vertebrate animal cell of claim 41 wherein the mammal is a murine species.

43. The genetically modified vertebrate animal cell of claim 41 wherein the mammal is a human.

44. The genetically modified vertebrate animal cell of claim 38 wherein the cell further comprises a nucleotide sequence complementary to said target endogenous nucleotide sequence.

45. The genetically modified vertebrate animal cell of claim 38 wherein the nucleotide sequences identical and complementary to said target endogenous nucleotide sequences are separated by an intron sequence.

46. The genetically modified vertebrate animal cell of claim 45 wherein the intron sequence is an intron from a gene encoding β-globin.

47. The genetically modified vertebrate animal cell of claim 46 wherein the β-globin intron is human β-globin intron 2.

48. A method of altering the phenotype of a vertebrate animal cell wherein said phenotype is conferred or otherwise facilitated by the expression of an endogenous gene, said method comprising introducing a genetic construct into said cell or a parent of said cell wherein the genetic construct comprises a nucleotide sequence substantially identical to a nucleotide sequence comprising said endogenous gene or part thereof and wherein a transcript resulting from transcription of said endogenous gene exhibits an altered capacity for translation into a proteinaceous product compared to a cell without having had the genetic construct introduced.

49. The method of claim 48 wherein the vertebrate animal cell is from a mammal, avian species, fish or reptile.

50. The method of claim 49 wherein the vertebrate animal cell is from a mammal.

51. The method of claim 50 wherein the mammal is a human, primate, livestock animal or laboratory test animal.

52. The method of claim 51 wherein the mammal is a murine species.

53. The method of claim 51 wherein the mammal is a human.

54. The method of claim 48 wherein the construct further comprises a nucleotide sequence complementary to said target endogenous nucleotide sequence.

55. The method of claim 48 wherein the nucleotide sequences identical and complementary to said target endogenous nucleotide sequences are separated by an intron sequence.

56. The method of claim 55 wherein the intron sequence is an intron from a gene encoding β-globin.

57. The method of claim 56 wherein the β-globin intron is human β-globin intron 2.

58. The genetically modified animal comprising the genetically modified vertebrate animal cells of claim 26.

59. The genetically modified animal comprising the genetically modified vertebrate animal cells of claim 38.

60. A genetically modified murine animal comprising a nucleotide sequence substantially identical to a target endogenous sequence of nucleotides in the genome of a cell of said murine animal wherein an RNA transcript resulting from transcription of a gene comprising said endogenous target sequence of nucleotides exhibits an altered capacity for translation into a proteinaceous product.

61. The genetically modified murine animal of claim 60 wherein the construct further comprises a nucleotide sequence complementary to said target endogenous nucleotide sequence.

62. The genetically modified murine animal of claim 60 wherein the nucleotide sequences identical and complementary to said target endogenous nucleotide sequences are separated by an intron sequence.

63. The genetically modified murine animal of claim 62 wherein the intron sequence is an intron from a gene encoding β-globin.

64. The genetically modified murine animal of claim 63 wherein the β-globin intron is human β-globin intron 2.

65. The genetically modified murine animal of claim 60 wherein there is substantially no reduction in the level of transcription of said gene comprising the endogenous target sequence.

66. The genetically modified murine animal of claim 60 wherein total level of RNA transcribed from said gene comprising said endogenous target sequence of nucleotides is not substantially reduced.

67. A method of generating a genetically modified vertebrate animal cell, said method comprising introducing into said animal cells a genetic construct comprising a sequence of nucleotides substantially identical to a target endogenous sequence of nucleotides in the genome of said vertebrate animal cells so upon transcription into RNA of a gene comprising said endogenous target sequence of nucleotides, the RNA transcript exhibits an altered capacity for translation into a proteinaceous product.

68. The method of claim 67 wherein the vertebrate animal cell is from a mammal, avian species, fish or reptile.

69. The method of claim 68 wherein the vertebrate animal cell is from a mammal.

70. The method of claim 69 wherein the mammal is a human, primate, livestock animal or laboratory test animal.

71. The method of claim 70 wherein the mammal is a murine species.

72. The method of claim 70 wherein the mammal is a human.

73. The of claim 67 wherein the construct further comprises a nucleotide sequence complementary to said target endogenous nucleotide sequence.

74. The method of claim 73 wherein the nucleotide sequences identical and complementary to said target endogenous nucleotide sequences are separated by an intron sequence.

75. The method of claim 74 wherein the intron sequence is an intron from a gene encoding β-globin.

76. The method of claim 75 wherein the β-globin intron is human β-globin intron 2.

77. The method of claim 67 wherein there is substantially no reduction in the level of transcription of said gene comprising the endogenous target sequence.

78. The method of claim 67 wherein total level of RNA transcribed from said gene comprising said endogenous target sequence of nucleotides is not substantially reduced.

79. A method of genetic therapy in a vertebrate animal, said method comprising introducing into cells of said animal a construct comprising a sequence of nucleotides substantially identical to a target endogenous sequence of nucleotides in the genome of said animal cells so upon transcription into RNA of a gene comprising said endogenous target sequence of nucleotides, the RNA transcript exhibits an altered capacity for translation into a proteinaceous product.

80. The method of claim 79 wherein the vertebrate animal is a mammal, avian species, fish or reptile.

81. The method of claim 80 wherein the vertebrate animal is a mammal.

82. The method of claim 81 wherein the mammal is a human, primate, livestock animal or laboratory test animal.

83. The method of claim 82 wherein the mammal is a murine species.

84. The method of claim 82 wherein the mammal is a human.

85. The method of claim 79 wherein said introduced nucleotide sequence further comprises a nucleotide sequence complementary to said target endogenous nucleotide sequence.

86. The method of claim 85 wherein the nucleotide sequences identical and complementary to said target endogenous nucleotide sequences are separated by an intron sequence.

87. The method of claim 86 wherein the intron sequence is an intron from a gene encoding β-globin.

88. The method of claim 87 wherein the β-globin intron is human β-globin intron 2.

89. The method of claim 79 wherein there is substantially no reduction in the level of transcription of said gene comprising the endogenous target sequence.

90. The method of claim 79 wherein total level of RNA transcribed from said gene comprising said endogenous target sequence of nucleotides is not substantially reduced.


SEQUENCE LISTING (25 sequences)

SEQ ID NO: 1 - DNA, 29 bp, Artificial Sequence
  Primer
gagctcttcagggtgagtctatgggaccc

SEQ ID NO: 2 - DNA, 29 bp, Artificial Sequence
  Primer
ctgcaggagctgtgggaggaagataagag

SEQ ID NO: 3 - DNA, 24 bp, Artificial Sequence
  Primer
tctccttacgcgtctgtgcggtat

SEQ ID NO: 4 - DNA, 24 bp, Artificial Sequence
  Primer
atgaggacacgtaggagcttcctg

SEQ ID NO: 5 - DNA, 30 bp, Artificial Sequence
  Primer
cccggggcttagtgtaaaacaggctgagag

SEQ ID NO: 6 - DNA, 30 bp, Artificial Sequence
  Primer
cccgggcaaatcccagtcatttcttagaaa

SEQ ID NO: 7 - DNA, 39 bp, Artificial Sequence
  Primer
cggcagatcctaacaatggcaggacaaatcgagtacatc

SEQ ID NO: 8 - DNA, 29 bp, Artificial Sequence
  Primer
gggcggatccttagaaagaatcgtaccac

SEQ ID NO: 9 - DNA, 20 bp, Artificial Sequence
  Primer
gtttccagatctctgatggc

SEQ ID NO: 10 - DNA, 20 bp, Artificial Sequence
  Primer
agtccactctggatcctagg

SEQ ID NO: 11 - DNA, 20 bp, Artificial Sequence
  Primer
cacagacagatctcttcagg

SEQ ID NO: 12 - DNA, 20 bp, Artificial Sequence
  Primer
actttagacggatccagcac

SEQ ID NO: 13 - DNA, 30 bp, Artificial Sequence
  Primer
agatctatttttccacccacggactctcgg

SEQ ID NO: 14 - DNA, 30 bp, Artificial Sequence
  Primer
ggatccgccacgaacaaggaagaaactagc

SEQ ID NO: 15 - DNA, 29 bp, Artificial Sequence
  Primer
ctcgagaagtgtgcaccggcacagacatg

SEQ ID NO: 16 - DNA, 29 bp, Artificial Sequence
  Primer
gtcgactgtgttccatcctctgctgtcac

SEQ ID NO: 17 - DNA, 30 bp, Artificial Sequence
  Primer
agatctgacagaaagagcgagcgaggagag

SEQ ID NO: 18 - DNA, 30 bp, Artificial Sequence
  Primer
ggattcagtgcgggtcgtggtgcgcgcctg

SEQ ID NO: 19 - DNA, 20 bp, Artificial Sequence
  Double Stranded DNA Probe
gcataattaatgaattagtg

SEQ ID NO: 20 - DNA, 23 bp, Artificial Sequence
  Double Stranded DNA Probe
gaagtatgcaaagcatgcatctc

SEQ ID NO: 21 - DNA, 23 bp, Artificial Sequence
  Double Stranded DNA Probe
gaagtaaggaaagcatgcatctc

SEQ ID NO: 22 - DNA, 30 bp, Artificial Sequence
  Primer
agatctgcagcagaccgtaaccattatagg

SEQ ID NO: 23 - DNA, 30 bp, Artificial Sequence
  Primer
ggatccacctttattaacaggtgcttgcag

SEQ ID NO: 24 - DNA, 30 bp, Artificial Sequence; identical to SEQ ID NO: 18
  Primer

SEQ ID NO: 25 - DNA, 30 bp, Artificial Sequence
  Primer
ggatcccaggccccactttcttgaccattg